(19) United States
(12) Patent Application Publication
(10) Pub. No.: US 2024/0390265 A1
(43) Pub. Date: Nov. 28, 2024

(54) FORMULATIONS FOR TRANSDERMAL ADMINISTRATION OF ACTIVE AGENTS

(71) Applicant: DYVE BIOSCIENCES, INC., Camarillo, CA (US)

(72) Inventors: NATHAN FITZSIMMONS, CAMARILLO, CA (US); RYAN BEAL, CAMARILLO, CA (US); CHARLES A. HARBERT, CAMARILLO, CA (US); AUDRENE RICE, CAMARILLO, CA (US); BRANDON SAND, CAMARILLO, CA (US); PARSHVA KOTHARY, CAMARILLO, CA (US)

(21) Appl. No.: 18/796,818

(22) Filed: Aug. 7, 2024

Related U.S. Application Data
(63) Continuation of application No. PCT/US23/12833, filed on Feb. 10, 2023.
(60) Provisional application No. 63/388,907, filed on Jul. 13, 2022.
(60) Provisional application No. 63/336,212, filed on Apr. 28, 2022.
(60) Provisional application No. 63/308,906, filed on Feb. 10, 2022.

(51) Int. Cl. A61K 9/00; A61K 31/5377; A61K 45/06; A61K 47/10; A61K 47/12; A61K 47/14; A61K 47/20; A61K 47/24; A61K 47/34

(52) U.S. Cl. CPC A61K 9/0014; A61K 31/5377; A61K 45/06; A61K 47/10; A61K 47/12; A61K 47/14; A61K 47/20; A61K 47/24; A61K 47/34

(57) ABSTRACT

The present disclosure relates to a formulations and methods for transdermal delivery of a medicament through the skin of a subject. In aspects, the formulation comprises a therapeutically effective amount of a medicament and a penetrant portion in which the penetrant portion comprises: a phospholipid, a fatty acid ester formed from a low molecular weight alcohol, and a long-chain fatty acid. In some embodiments, the penetrant portion further comprises one or more of a viscosity-improving agents, a penetration enhancer, and an emulsifier.

### CROSS-REFERENCE TO RELATED APPLICATIONS

This application is a continuation of international application PCT/US23/12833 filed on Feb. 10, 2023, which claims the benefit of U.S. Provisional Patent Application No. 63/308,906, filed Feb. 10, 2022; U.S. Provisional Patent Application No. 63/336,212, filed Apr. 28, 2022; and U.S. Provisional Patent Application No. 63/388,907, filed Jul. 13, 2022. The entire contents of the aforementioned patent applications are incorporated herein by reference.

### BACKGROUND

Topical administration describes the application of a substance to a surface of the skin. The term is often used to describe the application of a cream, foam, gel, lotion or ointment to the skin or mucous membranes. The high keratinization of skin cells and their dense packing creates, in most cases, a barrier impermeable to penetration. Because of this, most substances are not absorbed through the skin. There is an unmet need for formulations and method for transdermal penetration that work with a variety of medicaments and active agents, which overcome the barrier presented by the stratum corneum as well as the deeper layers of skin, that do so without harsh solvents, and are effective in delivering high molecular weight agents such as peptides, proteins and nucleic acids.

### SUMMARY

Aspects of the present disclosure teach certain benefits in construction and use which give rise to the exemplary advantages described below.

The present disclosure relates to systems and methods of transdermal administration of medicaments for a range of benefits.

The present disclosure solves the problems described above by providing transdermal delivery formulations with improved penetration. In at least one embodiment, disclosed herein are transdermal penetrant formulations for transdermal administration of a medicament.

An aspect of the present disclosure is a formulation for transdermal delivery of a medicament through the skin of a subject. The formulation comprises a therapeutically effective amount of a medicament and a penetrant portion. The penetrant portion comprises: a phospholipid, a fatty acid ester formed from a low molecular weight alcohol, and a long-chain fatty acids, and, optionally, one or more of a viscosity-improving agents, a penetration enhancer, and an emulsifier.

In embodiments, the phospholipid is selected from phosphatidylcholine, hydrogenated phosphatidylcholine, phosphatidylserine, phosphatidylethanolamine, phosphatidylinositol, inositol phosphatide, and sphingomyelin. In some cases, the phospholipid is phosphatidylcholine.

In some embodiments, the penetrant portion comprises two or more phospholipids.

In various embodiments, the phospholipid is in an amount from about 3% to about 15% w/w of the formulation.

In certain embodiments, the low molecular weight alcohol is selected from isopropanol, methanol, ethanol, butanol, glycerol, cetyl alcohol.

In embodiments, the low molecular weight alcohol is isopropanol.

In some embodiments, the fatty acid ester is selected from isopropyl palmitate, isopropyl myristate, isopropyl linoleate, isopropyl oleate, ethyl laurate, and ethyl myristate. In some cases, the fatty acid ester is isopropyl palmitate.

In various embodiments, the penetrant portion comprises two or more fatty acid esters.

In certain embodiments, the fatty acid ester is in an amount from about 5% to about 20% w/w of the formulation.

In embodiments, the long-chain fatty acid is selected from a linoleic, oleic, stearic acid, linolenic, palmitic, arachidonic, palmitoleic, myristic, eicosenoic, benehic, euricic, and lignoceric acid. In some cases, the long-chain fatty acid is linoleic acid. In other cases, the long-chain fatty acid is oleic acid. In further cases, the long-chain fatty acid is stearic acid. In some cases, the long-chain fatty acid is obtained from safflower oil or almond oil.

In some embodiments, the long-chain fatty acid is in an amount from about 0.1% to about 10% w/w of the formulation.

In various embodiments, the penetrant portion comprises two or more long-chain fatty acids.

In certain embodiments, the penetrant portion comprises a viscosity-improving agent.

In embodiments, the viscosity-improving agent is a poloxamer. In some cases, the poloxamer is selected from poloxamer 407, poloxamer 188, poloxamer 184, and poloxamer 124.

In some embodiments, the viscosity-improving agent is a surfactant. In some cases, the surfactant is selected from sodium lauryl sulfate (sodium dodecyl sulfate); polyoxyethylated castor oil derivatives such as HCO-60 surfactant; nonoxynol; octoxynol; phenylsulfonate; poloxamers such as Pluronic® F68, Pluronic® F127, and Pluronic® L62; polyoleates; Rewopal® HVIO, sodium laurate, sodium oleate; sorbitan dilaurate; sorbitan dioleate; sorbitan monolaurate such as Span® 20; sorbitan monooleates; sorbitan trilaurate; sorbitan trioleate; sorbitan monopalmitate such as Span® 40; sorbitan stearate such as Span® 85; polyethylene glycol nonylphenyl ether such as Synperonic® NP; p-(1,1,3,3-tetramethylbutyl)-phenyl ether such as Triton™ X-100; and polysorbates such as polyoxyethylene (20) sorbitan monolaurate such as Tween® 20, polysorbate 40 (polyoxyethylene (20) sorbitan monopalmitate) such as Tween® 40, polysorbate 60 (polyoxyethylene (20) sorbitan monostearate) such as Tween® 60, polysorbate 80 (polyoxyethylene (20) sorbitan monooleate) such as Tween® 80, and polyoxyethylenesorbitan trioleate such as Tween® 85. In some cases, the surfactant is sodium lauryl sulfate.

In embodiments, the penetrant portion comprises two or more viscosity-improving agents.

In certain embodiments, the viscosity-improving agent is in an amount from about 5% to about 20% w/w of the formulation.

In embodiments, the penetrant portion comprises a penetration enhancer. In some embodiments, the penetration enhancer is an alcohol or a terpene. In some cases, the penetration enhancer as an alcohol is selected from benzyl alcohol, ethanol, propylene glycol, and polyethylene glycol. In various cases, the penetration enhancer is benzyl alcohol. In some cases, the penetration enhancer as a terpene is selected from limonene, menthol, borneol, and camphor. In various embodiments, the penetration enhancer further acts as a preservative.

In some embodiments, the penetrant portion comprises two or more penetration enhancers.

In various embodiments, the penetration enhancer is in an amount from about 0.5% to about 5% w/w of the formulation.

In certain embodiments, the penetrant portion comprises at least one penetration enhancer and at least one viscosity-improving agent.

In embodiments, the penetrant portion comprises an emulsifier.

In some embodiments, the emulsifier is selected from polyglyceryl-4-laurate, polyglyceryl-4-oleate, span 60, cetyl alcohol, and polyglyceryl-3-oleate.

In various embodiments, the penetrant portion comprises two or more penetration enhancers.

In certain embodiments, the emulsifier is in an amount from about 0.5 to about 10% w/w of the formulation.

In embodiments, the penetrant portion comprises at least one emulsifier and at least one viscosity-improving agent.

In some embodiments, the penetrant portion comprises at least one emulsifier and at least one penetration enhancer.

In various embodiments, the penetrant portion comprises at least one emulsifier, at least one viscosity-improving agent, and at least one penetration enhancer.

Another aspect of the present disclosure is a formulation for transdermal delivery of a medicament through the skin of a subject. The formulation comprises a therapeutically effective amount of a medicament and a penetrant portion in which the penetrant portion comprises: a phospholipid, a fatty acid ester formed from a low molecular weight alcohol, and a long-chain fatty acids. In this aspect, phosphatidylcholine, hydrogenated phosphatidylcholine, phosphatidylserine, phosphatidylethanolamine, phosphatidylinositol, inositol phosphatide, or sphingomyelin is the phospholipid; isopropyl palmitate, isopropyl myristate, isopropyl linoleate, isopropyl oleate, ethyl laurate, or ethyl myristate is the fatty acid ester; and a linoleic, oleic, stearic acid, linolenic, palmitic, arachidonic, palmitoleic, myristic, eicosenoic, benehic, euricic, or lignoceric acid is the long-chain fatty acid or the long-chain fatty acid is obtained from safflower oil or almond oil.

A further aspect of the present disclosure is a formulation for transdermal delivery of a medicament through the skin of a subject. The formulation comprises a therapeutically effective amount of a medicament and a penetrant portion in which the penetrant portion comprises: a phospholipid, a fatty acid ester formed from a low molecular weight alcohol, and a long-chain fatty acids, and one or more of a viscosity-improving agents, a penetration enhancer, and an emulsifier. In this aspect, phosphatidylcholine, hydrogenated phosphatidylcholine, phosphatidylserine, phosphatidylethanolamine, phosphatidylinositol, inositol phosphatide, or sphingomyelin is the phospholipid; isopropyl palmitate, isopropyl myristate, isopropyl linoleate, isopropyl oleate, ethyl laurate, or ethyl myristate is the fatty acid ester; and a linoleic, oleic, stearic acid, linolenic, palmitic, arachidonic, palmitoleic, myristic, eicosenoic, benehic, euricic, or lignoceric acid is the long-chain fatty acid or the long-chain fatty acid is obtained from safflower oil or almond oil; polyglyceryl-4-laurate, polyglyceryl-4-oleate, span 60, cetyl alcohol, or polyglyceryl-3-oleate is the penetration enhancer; a poloxamer (e.g., poloxamer 407, poloxamer 188, poloxamer 184, and poloxamer 124) or sodium lauryl sulfate is the viscosity-improving agent; benzyl alcohol, ethanol, propylene glycol, polyethylene glycol, limonene, menthol, borneol, or camphor is the penetration enhancer.

In certain embodiments, the penetrant portion is in an amount from about 70% to about 98% w/w of the formulation.

In embodiments, the penetrant portion comprises water.

In some embodiments, the penetrant portion comprises water in an amount from about 50% to about 80% w/w of the formulation.

In various embodiments, the formulation comprises a phospholipid, an emollient/moisturizer, a fatty acid, an alcohol, an oil, a surfactant, water, and a medicament.

An additional aspect of the present disclosure is a formulation for transdermal delivery of a medicament through the skin of a subject. The formulation comprises a phospholipid in an amount from about 5% to about 15% w/w of the formulation; an emollient/moisturizer in an amount from about 10% to about 20% w/w of the formulation; a fatty acid in an amount from about 0.5% to about 2% w/w of the formulation; an alcohol in an amount from about 0.5% to about 2% w/w of the formulation; an oil in an amount from about 1% to about 5% w/w of the formulation; a surfactant in an amount from about 0.5% to about 2% w/w of the formulation; water in an amount from about 30% to about 80% w/w of the formulation; and a therapeutically effective amount of a medicament in an amount from about 0.001% to about 30% w/w of the formulation. In some cases, the medicament is in an amount from about 0.001% to about 0.01% w/w of the formulation. In other cases, the medicament is in an amount from about 0.011% to about 0.1% w/w of the formulation. In various cases, the medicament is in an amount from about 0.11% to about 1.0% w/w of the formulation. In further cases, the medicament is in an amount from about 1% to about 10% w/w of the formulation. In additional cases, the medicament is in an amount from about 11% to about 20% w/w of the formulation. In alternate cases, the medicament is in an amount from about 21% to about 30% w/w of the formulation.

In an aspect, the present disclosure provides a formulation for transdermal delivery of a medicament through the skin of a subject. The formulation comprises phosphatidylcholine in an amount of about 7.64% w/w of the formulation; isopropyl palmitate in an amount of about 13.30% w/w of the formulation; stearic acid in an amount of about 0.62% w/w of the formulation; benzyl alcohol in an amount of about 1.39% w/w of the formulation; safflower oil in an amount of about 2.93% w/w of the formulation; oleic acid in an amount of about 0.97% w/w of the formulation; polyglyceryl-4 laurate in an amount of about 1.06% w/w of the formulation; deionized water in an amount of about 60.84% w/w of the formulation; poloxamer 407 in an amount of about 9.25% w/w of the formulation; and a therapeutically effective amount of a medicament in an amount of about 2% w/w of the formulation. In some cases, rather than the medicament being in an amount of about 2% w/w of the formulation, the amount of the medicament is less than about 2% and the amount of water is increased proportionally. In other cases, rather than the medicament being in an amount of about 2% w/w of the formulation, the amount of the medicament is greater than about 2% and the amount of water is decreased proportionally. In various cases, the amount of the medicament is less than about 30%.

In another aspect, the present disclosure provides a formulation for transdermal delivery of a medicament through the skin of a subject. The formulation comprises phosphatidylcholine in an amount of about 7.66% w/w of the formulation; isopropyl palmitate in an amount of about 13.34% w/w of the formulation; benzyl alcohol in an amount of about 1.39% w/w of the formulation; stearic acid in an amount of about 0.68% w/w of the formulation; carthamus tinctorius (safflower) oil in an amount of about 2.79% w/w of the formulation; polyglyceryl-4 laurate in an amount of about 1.07% w/w of the formulation; oleic acid in an amount of about 1.06% w/w of the formulation; deionized water in an amount of about 61.73% w/w of the formulation; poloxamer 407 in an amount of about 9.28% w/w of the formulation; and a therapeutically effective amount of a medicament in an amount of about 1.00% w/w of the formulation. In some case, rather than the medicament being in an amount of about 1% w/w of the formulation, the amount of the medicament is less than about 1% and the amount of water is increased proportionally. In other cases, rather than the medicament being in an amount of about 1% w/w of the formulation, the amount of the medicament is greater than about 1% and the amount of water is decreased proportionally. In various cases, the amount of the medicament is less than about 30%.

In a further aspect, the present disclosure provides a formulation for transdermal delivery of a medicament through the skin of a subject. The formulation comprises a therapeutically effective amount of a medicament and a penetrant portion in which the penetrant portion comprises: phosphatidylcholine in an amount from about 3% to about 15% w/w of the formulation; isopropyl palmitate in an amount from about 5% to about 20% w/w of the formulation; stearic acid in an amount from about 0.1% to about 10% w/w of the formulation; benzyl alcohol in an amount from about 0.5% to about 5% w/w of the formulation; polyglyceryl-4 laurate in an amount from about 0.5% to about 10% w/w of the formulation; and poloxamer 407 in an amount from about 5% to about 20% w/w of the formulation.

In certain embodiments, the formulation has a pH from about 7 to about 10.5.

In embodiments, the formulation has a pH from about 9 to about 11.

In embodiments, the transdermal delivery formulations of the present disclosure deliver medicaments of various sizes. As examples, the medicaments having a molecular weight of less than about 500 Da, e.g., Nicotine (162.2 Da), Diphenhydramine Hydrochloride (291.8 Da), and Hydrocortisone (362.5 Da); medicaments having a molecular weight from about 500 Da to about 1000 Da, e.g., Sildenafil Citrate (666.7 Da), Neratinib (557 Da), and Doxycycline hyclate (512.9 Da); and medicaments having a molecular weight greater than about 1000 Da, e.g., Cyclosporine A (1202.6 Da), Vancomycin Hydrochloride (1485.70 Da), and RBD protein (10,000+Da).

In various embodiments, the medicament is at least one of Polidocanol, MRTX1133, Resveratrol, LUT014, Incobotulinum toxin A to Incobotulinum toxin G, a nucleic acid, Naporafenib, or one or more herein-disclosed buffering agents.

In embodiments, the medicament is a nucleic acid. Examples of nucleic acids useful in formulations of the present disclosure include a double-stranded DNA (dsDNA) molecule, a single-stranded DNA (ssDNA) molecule, a dsRNA molecule, a ssRNA molecule, a plasmid, an oligonucleotide, a synthetic RNA molecule, a miRNA molecule, an mRNA molecule, and an siRNA molecule. In some cases, the nucleic acid is an mRNA molecule, e.g., that encodes a polypeptide or protein.

In some embodiments, one or more medicaments is selected from Table 1 or comprises buffering agents as disclosed herein. In some embodiments, the amount of the one or more medicaments is the effective dose of the medicament as described in Table 1 or the effective dose of the buffering agents as disclosed herein.

In various embodiments, the medicament is a buffering agent. In some embodiments, the buffering agent is Sodium Hydroxide (Sodium oxidanide), Sodium Bicarbonate (baking soda or Sodium hydrogen carbonate), Potassium Bicarbonate (potassium hydrogen carbonate or potassium acid carbonate), Lysine, Tris (Tromethamine, trisaminomethane, 2-amino-2-hydroxymethyl-propane-1,3-diol, or tris(hydroxymethyl)aminomethane), Calcium Carbonate, Sodium Carbonate (Disodium carbonate), Potassium Carbonate, Dipotassium Phosphate (Potassium phosphate dibasic or Potassium hydrogen phosphate), Disodium Phosphate (Sodium phosphate dibasic or Disodium hydrogen phosphate), Trisodium Phosphate, Meglumine ((2R,3R,4R,5S)-6-(Methylamino)hexane-1,2,3,4,5-pentol or Methylglucamine), Arginine, Triethanolamine (TEA or 2,2′,2″-Nitrilotriethanol), Glycine, Monosodium Phosphate (Sodium dihydrogen phosphate), Monopotassium Phosphate (Potassium dihydrogen phosphate), Tripotassium Phosphate (potassium phosphate), Monoethanolamine, Diethanolamine (Diolamine or 2-(2-hydroxyethylamino)ethanol), Magnesium carbonate, 2-imidazole-1-yl-3-ethoxycarbonylpropionic acid (IEPA), or combination thereof. Buffering agents are especially useful medicaments for treating cancer. Buffering agents are especially useful medicaments for treating diseases or disorders that are local and/or superficial (e.g., relative to the skin surface), local, non-superficial (e.g., relative to the skin surface), or systemic disease with an inflammatory aspect, as disclosed elsewhere herein.

Using non-sodium buffering agents may help avoid hypernatremia, which is a high concentration of sodium in the blood, in a subject which could result when a high dosage of sodium-based buffering agent is administered and/or for a long period of time. In transdermal delivery formulations of the present disclosure comprising buffering agents, a combination of non-sodium and sodium buffering agents can be used. In some embodiments, the amount of the sodium salt in the buffering agent is 1%, 2%, 3%, 4%, 5%, 6%, 7%, 8%, 9%, 10%, 20%, 30%, 40%, 50%, 60%, 70%, or so of the amount of the non-sodium salts in the buffering agent.

In any herein disclosed aspect or embodiment, transdermal delivery provides systemic administration of a medicament.

In yet another aspect, the present disclosure provides a method for transdermally delivering at least one medicament. The method comprises a step of applying to the skin of a subject an effective amount of any herein disclosed formulation.

An aspect of the present disclosure is a method for treating a disease or disorder or reducing a symptom thereof. The method comprises steps of administering to a subject in need thereof any herein disclosed transdermal delivery formulation and administering to the subject in need thereof a composition comprising one or more medicaments selected from Table 1. In some case, the transdermal delivery formulation is administered before, contemporary with, or after the composition is administered. In various cases, the amount of the one or more medicaments is the effective dose of the medicament as described in Table 1. In some cases, the composition is administered by the standard route for the medicament. In some cases, the standard route is oral, topical, enteral, parenteral, by intravenous injection or infusion, by intraperitoneal injection, by intramuscular injection, or by subcutaneous injection. In various cases, the composition is a liquid, a suspension, a gel, a geltab, a semisolid, a tablet, a sachet, a lozenge, a pill, or a capsule.

Another aspect of the present disclosure is a use of any herein disclosed transdermal delivery formulation in a method for treating a disease or disorder or reducing a symptom thereof.

A further aspect of the present disclosure is a method for manufacturing a medicament for treating a disease or disorder or reducing a symptom thereof comprising combining a penetrant portion as recited in any herein disclosed formulation with one or more medicaments recited in Table 1 or with one or more buffering agents as disclosed herein.

Aspects disclosed herein provide a method of treating cancer comprising: applying the transdermal delivery formulation to the skin of a subject an effective amount of any herein disclosed formulation, wherein the formulation comprises buffering agent, to a skin of a subject; increasing a pH of a tumor microenvironment; increasing one or more of: translation of a proinflammatory cytokines T cell activation; perfusion, or activity, of an immunotherapy, immunomodulatory agent, or chemotherapeutic agent within the tumor microenvironment; and decreasing one or more of: tumor size, or inhibition of glycolytic activity. In some embodiments, the buffering agent is Sodium Hydroxide (Sodium oxidanide), Sodium Bicarbonate (baking soda or Sodium hydrogen carbonate), Potassium Bicarbonate (potassium hydrogen carbonate or potassium acid carbonate), Lysine, Tris (Tromethamine, trisaminomethane, 2-amino-2-hydroxymethyl-propane-1,3-diol, or tris(hydroxymethyl)aminomethane), Calcium Carbonate, Sodium Carbonate (Disodium carbonate), Potassium Carbonate, Dipotassium Phosphate (Potassium phosphate dibasic or Potassium hydrogen phosphate), Disodium Phosphate (Sodium phosphate dibasic or Disodium hydrogen phosphate), Trisodium Phosphate, Meglumine ((2R,3R,4R,5S)-6-(Methylamino)hexane-1,2,3,4,5-pentol or Methylglucamine), Arginine, Triethanolamine (TEA or 2,2′,2″-Nitrilotriethanol), Glycine, Monosodium Phosphate (Sodium dihydrogen phosphate), Monopotassium Phosphate (Potassium dihydrogen phosphate), Tripotassium Phosphate (potassium phosphate), Monoethanolamine, Diethanolamine (Diolamine or 2-(2-hydroxyethylamino)ethanol), Magnesium carbonate, 2-imidazole-1-yl-3-ethoxycarbonylpropionic acid (IEPA), or combination thereof. In some embodiments, the cancer or the tumor microenvironment is near the surface of the skin. In some embodiments, the cancer or the tumor microenvironment comprises a melanoma.

Aspects disclosed herein provide a method of treating or preventing a symptom of an EGFR inhibitor induced side effect, the method comprising: applying the transdermal delivery formulation to the skin of a subject an effective amount of any herein disclosed formulation, wherein the formulation comprises LUT014, to a skin of a subject; binding BRAF with LUT014 or a metabolite thereof, thereby inhibiting an MAP Kinase pathway; and treating or preventing the symptom of the EGFR inhibitor induced side effect. In some embodiments, the EGFR inhibitor induced side effect comprises acneiform lesions.

Aspects disclosed herein provide a method of treating or preventing a symptom of radiation dermatitis, the method comprising: applying the transdermal delivery formulation to the skin of a subject an effective amount of any herein disclosed formulation, wherein the formulation comprises LUT014, to a skin of a subject; binding BRAF with LUT014 or a metabolite thereof, thereby increasing a prefoliation of a keratinocyte; and treating or preventing the symptom of radiation dermatitis. In some embodiments, treating or preventing the symptom of radiation dermatitis comprises decreasing in collagen production, improving skin elasticity, reducing telangiectasia, reducing redness, reducing inflammation, reducing swelling, reducing blisters or skin ulcers, reducing thinning or weakening of the skin, or combinations thereof.

Aspects disclosed herein provide a method of improving wound healing, the method comprising: applying the transdermal delivery formulation to the skin of a subject an effective amount of any herein disclosed formulation, wherein the formulation comprises LUT014, to a skin of a subject; binding BRAF with LUT014 or a metabolite thereof, thereby inhibiting an MAP Kinase pathway; and improving wound healing.

Other aspects disclosed herein provide a method of vaccinating a subject. The method comprising a step of applying the transdermal delivery formulation to the skin of a subject an effective amount of any herein disclosed formulation, wherein the formulation comprises a nucleic acid. In embodiments, the nucleic acid is an mRNA molecule, e.g., encodes a polypeptide or protein.

Any aspect or embodiment described herein can be combined with any other aspect or embodiment as disclosed herein.

### DETAILED DESCRIPTION

### Introduction

The term transdermal administration refers to applying a substance onto the skin so that it is absorbed into the body for local or systemic distribution. A transdermal solution (e.g., cream, ointment, or lotion) or transdermal patch is typically placed on one's skin. The solution or patch includes a medicament that is released into the skin. As the layers of skin absorb the solution, the medicament is absorbed via the blood vessels into the bloodstream. From there, the substance can be circulated through the body.

There are obvious advantages to transdermal administration of medicaments. The consumer does not have to schedule and remember to consume doses of pills. Further, transdermal administration is not affected by stomach or digestive issues. Administration across the skin enables drugs to avoid degradation in the gastrointestinal tract or liver. Transdermal delivery is therefore of particular interest for molecules with limited systemic bioavailabilities and short half-lives. Drugs that are absorbed slowly can be more effective. With a transdermal patch or cream, a medicament can be released in small quantities over a long period of time.

Transdermal administration can be effective in administering hydrophobic chemicals such as steroid hormones. For example, transdermal patches are a common means of administering steroidal drugs for birth control, hormone replacement therapy and prevention of motion sickness. Common medicaments that can be administered by transdermal patches include pain relievers, nicotine, hormones, and drugs to treat angina and motion sickness.

Medicaments that are not hydrophobic chemicals are typically unsuited for topical administration. To be effective, the active drug or agent in a topical composition must penetrate the skin, which is structurally complex and relatively thick. Molecules moving through the skin must first penetrate the stratum corneum and any material on its surface. The molecules must then penetrate the epidermis, the papillary dermis, and the capillary walls into the vascular system or lymphatic system. To be absorbed, the molecules must overcome a different resistance to penetration in each layer.

Strategies have been devised to improve transdermal administration of medicaments. These strategies can be categorized as either physical, chemical, mechanical or biochemical. Combinations of these strategies can also increase efficacy or extend the time for transdermal delivery. Physical techniques include abrasion and tape stripping, which physically break open the skin. Another physical method is prolonged occlusion, which alters the barrier properties of the stratum corneum. After 24 to 28 hours of occlusion with resultant hydration, corneocytes swell, intercellular spaces become distended, and the lacunar network becomes dilated. Distention of the lacunae eventually leads to connections with an otherwise discontinuous system. This creates pores in the stratum corneum interstices through which polar and non-polar substances can penetrate more easily.

Other approaches include the use of Chemical Permeation Enhancers. Chemical Permeation Enhancers (CPEs) are molecules that interact with the constituents of skin's outermost layer, the stratum corneum (SC), and increase its permeability. However, despite efforts at improving them, CPEs are minimally effective in increasing the rate at which drugs permeate the skin. CPEs can also cause skin damage, irritation and sensitization. Further, they are generally ineffective with high molecular weight drugs such as peptides, proteins and nucleic acids.

Although a variety of methods can be used to enhance transdermal drug delivery, these methods have limitations. Most efforts to enhance transdermal penetration have focused on the outermost layer of the skin, the stratum corneum. They typically rely on harsh solvents (e.g., alcohols, DMSO) or patch-based systems. This approach limits the molecular size, lipophilicity, and potency of drugs that can be used. In essence, current approaches are largely limited to small, lipophilic, and highly potent drugs. Illustrative aspects of the present disclosure.

The present disclosure provides improved formulations and method for transdermal penetration that work with a variety of medicaments and active agents. These formulations and methods overcome the barrier presented by the stratum corneum as well as the deeper layers of skin. Further, it does so without harsh solvents and are effective in delivering high molecular weight agents such as peptides, proteins and nucleic acids.

An aspect of the present disclosure is a formulation for transdermal delivery of a medicament through the skin of a subject. The formulation comprises a therapeutically effective amount of a medicament and a penetrant portion. The penetrant portion comprises: a phospholipid, a fatty acid ester formed from a low molecular weight alcohol, and a long-chain fatty acids, and, optionally, one or more of a viscosity-improving agents, a penetration enhancer, and an emulsifier.

Another aspect of the present disclosure is a formulation for transdermal delivery of a medicament through the skin of a subject. The formulation comprises a therapeutically effective amount of a medicament and a penetrant portion in which the penetrant portion comprises: a phospholipid, a fatty acid ester formed from a low molecular weight alcohol, and a long-chain fatty acid. In this aspect, phosphatidylcholine, hydrogenated phosphatidylcholine, phosphatidylserine, phosphatidylethanolamine, phosphatidylinositol, inositol phosphatide, or sphingomyelin is the phospholipid; isopropyl palmitate, isopropyl myristate, isopropyl linoleate, isopropyl oleate, ethyl laurate, or ethyl myristate is the fatty acid ester; and a linoleic, oleic, stearic acid, linolenic, palmitic, arachidonic, palmitoleic, myristic, eicosenoic, benehic, euricic, or lignoceric acid is the long-chain fatty acid or the long-chain fatty acid is obtained from safflower oil or almond oil.

A further aspect of the present disclosure is a formulation for transdermal delivery of a medicament through the skin of a subject. The formulation comprises a therapeutically effective amount of a medicament and a penetrant portion in which the penetrant portion comprises: a phospholipid, a fatty acid ester formed from a low molecular weight alcohol, and a long-chain fatty acids, and one or more of a viscosity-improving agents, a penetration enhancer, and an emulsifier. In this aspect, phosphatidylcholine, hydrogenated phosphatidylcholine, phosphatidylserine, phosphatidylethanolamine, phosphatidylinositol, inositol phosphatide, or sphingomyelin is the phospholipid; isopropyl palmitate, isopropyl myristate, isopropyl linoleate, isopropyl oleate, ethyl laurate, or ethyl myristate is the fatty acid ester; and a linoleic, oleic, stearic acid, linolenic, palmitic, arachidonic, palmitoleic, myristic, eicosenoic, benehic, euricic, or lignoceric acid is the long-chain fatty acid or the long-chain fatty acid is obtained from safflower oil or almond oil; polyglyceryl-4-laurate, polyglyceryl-4-oleate, span 60, cetyl alcohol, or polyglyceryl-3-oleate is the penetration enhancer; a poloxamer (e.g., poloxamer 407, poloxamer 188, poloxamer 184, and poloxamer 124) or sodium lauryl sulfate is the viscosity-improving agent; benzyl alcohol, ethanol, propylene glycol, polyethylene glycol, limonene, menthol, borneol, or camphor is the penetration enhancer.

An additional aspect of the present disclosure is a formulation for transdermal delivery of a medicament through the skin of a subject. The formulation comprises a phospholipid in an amount from about 5% to about 15% w/w of the formulation; an emollient/moisturizer in an amount from about 10% to about 20% w/w of the formulation; a fatty acid in an amount from about 0.5% to about 2% w/w of the formulation; an alcohol in an amount from about 0.5% to about 2% w/w of the formulation; an oil in an amount from about 1% to about 5% w/w of the formulation; a surfactant in an amount from about 0.5% to about 2% w/w of the formulation; water in an amount from about 30% to about 80% w/w of the formulation; and a therapeutically effective amount of a medicament in an amount from about 0.001% to about 30% w/w of the formulation. In some cases, the medicament is in an amount from about 0.001% to about 0.01% w/w of the formulation. In other cases, the medicament is in an amount from about 0.011% to about 0.1% w/w of the formulation. In various cases, the medicament is in an amount from about 0.11% to about 1.0% w/w of the formulation. In further cases, the medicament is in an amount from about 1% to about 10% w/w of the formulation. In additional cases, the medicament is in an amount from about 11% to about 20% w/w of the formulation. In alternate cases, the medicament is in an amount from about 21% to about 30% w/w of the formulation.

In an aspect, the present disclosure provides a formulation for transdermal delivery of a medicament through the skin of a subject. The formulation comprises phosphatidylcholine in an amount of about 7.64% w/w of the formulation; isopropyl palmitate in an amount of about 13.30% w/w of the formulation; stearic acid in an amount of about 0.62% w/w of the formulation; benzyl alcohol in an amount of about 1.39% w/w of the formulation; safflower oil in an amount of about 2.93% w/w of the formulation; oleic acid in an amount of about 0.97% w/w of the formulation; polyglyceryl-4 laurate in an amount of about 1.06% w/w of the formulation; deionized water in an amount of about 60.84% w/w of the formulation; poloxamer 407 in an amount of about 9.25% w/w of the formulation; and a therapeutically effective amount of a medicament in an amount of about 2% w/w of the formulation. In some cases, rather than the medicament being in an amount of about 2% w/w of the formulation, the amount of the medicament is less than about 2% and the amount of water is increased proportionally. In other cases, rather than the medicament being in an amount of about 2% w/w of the formulation, the amount of the medicament is greater than about 2% and the amount of water is decreased proportionally. In various cases, the amount of the medicament is less than about 30%.

In another aspect, the present disclosure provides a formulation for transdermal delivery of a medicament through the skin of a subject. The formulation comprises phosphatidylcholine in an amount of about 7.66% w/w of the formulation; isopropyl palmitate in an amount of about 13.34% w/w of the formulation; benzyl alcohol in an amount of about 1.39% w/w of the formulation; stearic acid in an amount of about 0.68% w/w of the formulation; carthamus tinctorius (safflower) oil in an amount of about 2.79% w/w of the formulation; polyglyceryl-4 laurate in an amount of about 1.07% w/w of the formulation; oleic acid in an amount of about 1.06% w/w of the formulation; deionized water in an amount of about 61.73% w/w of the formulation; poloxamer 407 in an amount of about 9.28% w/w of the formulation; and a therapeutically effective amount of a medicament in an amount of about 1.00% w/w of the formulation. In some case, rather than the medicament being in an amount of about 1% w/w of the formulation, the amount of the medicament is less than about 1% and the amount of water is increased proportionally. In other cases, rather than the medicament being in an amount of about 1% w/w of the formulation, the amount of the medicament is greater than about 1% and the amount of water is decreased proportionally. In various cases, the amount of the medicament is less than about 30%.

In a further aspect, the present disclosure provides a formulation for transdermal delivery of a medicament through the skin of a subject. The formulation comprises a therapeutically effective amount of a medicament and a penetrant portion in which the penetrant portion comprises: phosphatidylcholine in an amount from about 3% to about 15% w/w of the formulation; isopropyl palmitate in an amount from about 5% to about 20% w/w of the formulation; stearic acid in an amount from about 0.1% to about 10% w/w of the formulation; benzyl alcohol in an amount from about 0.5% to about 5% w/w of the formulation; polyglyceryl-4 laurate in an amount from about 0.5% to about 10% w/w of the formulation; and poloxamer 407 in an amount from about 5% to about 20% w/w of the formulation.

In yet another aspect, the present disclosure provides a method for transdermally delivering at least one medicament. The method comprises a step of applying to the skin of a subject an effective amount of any herein disclosed formulation.

An aspect of the present disclosure is a method for treating a disease or disorder or reducing a symptom thereof. The method comprises steps of administering to a subject in need thereof any herein disclosed transdermal delivery formulation and administering to the subject in need thereof a composition comprising one or more medicaments selected from Table 1. In some case, the transdermal delivery formulation is administered before, contemporary with, or after the composition is administered. In various cases, the amount of the one or more medicaments is the effective dose of the medicament as described in Table 1. In some cases, the composition is administered by the standard route for the medicament. In some cases, the standard route is oral, topical, enteral, parenteral, by intravenous injection or infusion, by intraperitoneal injection, by intramuscular injection, or by subcutaneous injection. In various cases, the composition is a liquid, a suspension, a gel, a geltab, a semisolid, a tablet, a sachet, a lozenge, a pill, or a capsule.

Another aspect of the present disclosure is a use of any herein disclosed transdermal delivery formulation in a method for treating a disease or disorder or reducing a symptom thereof.

A further aspect of the present disclosure is a method for manufacturing a medicament for treating a disease or disorder or reducing a symptom thereof comprising combining a penetrant portion as recited in any herein disclosed formulation with one or more medicaments recited in Table 1 or one or more buffering agents as disclosed herein.

Any aspect or embodiment described herein can be combined with any other aspect or embodiment as disclosed herein.

### Transdermal Delivery Formulation Components

Embodiments include a transdermal lotion or cream for administration of medicaments to a subject. It is placed on the skin to deliver a specific dose of an agent through the skin. The agent can be delivered across the skin into a localized subdermal location. For example, a lotion can alleviate inflammation from an autoimmune response. The lotion or cream can be applied directly to the affected area. Alternatively, the agent can enter the circulation for systemic distribution.

In an alternative embodiment, an agent can be administered using a transdermal or medicated adhesive patch. To release an agent, a patch can utilize a porous membrane covering a reservoir of the agent. Alternatively, the agent can be embedded in layers of the adhesive that release the agent as they dissolve or melt.

An advantage of a transdermal drug delivery route over other types of delivery is that the formulation can provide a controlled release of the agent. Conventional transdermal delivery systems are generally ineffective for use with agents and medications that are large molecules and/or hydrophilic molecules.

There are other advantages to transdermal administration of medicaments. Proteins and peptides used, for example, in aging treatments can be degraded by the gastric acid and enzymes. Transdermal administration is not affected by stomach or digestive issues. Further, people can benefit from drugs that are absorbed slowly and regularly. With a transdermal delivery formulation, a medicament can be released in small quantities over a long period of time.

Other advantages are related to dosing. Large doses of agents can cause dose-dependent toxicity in many cases. For example, oral administration of vitamin A can result in hypervitaminosis A. The main problems associated with the vitamin A are its half-life, fast absorption (due to lipophilicity) and its toxicity (due to high loading and frequent dosing). Also, some drugs undergo first-pass metabolism, which prevents their delivery to the desired site of action. Furthermore, many hydrophilic or lipophilic drugs show either poor dissolution or poor absorption on oral administration. With a transdermal delivery formulation, the effective concentration of an agent can be applied at the desired site without painful delivery.

Additionally, the transdermal delivery formulations of the present disclosure are able to deliver an active agent into an animal's bloodstream and thereby provide systemic administration of the agent (e.g., a medicament disclosed in Table 1 or one or more buffering agents as disclosed herein). In particular, the transdermal delivery formulations of the present disclosure can provide a higher concentration of a molecule (especially one that is insoluble) and can provide systemic administration for a molecule that would be poorly absorbed by the gut epithelium. Together, molecules that are not suitable for enteral delivery or are suitable but at a low dosage can be systemically administered via transdermal delivery formulations of the present disclosure.

In one embodiment, medicaments are supplied via transdermal administration. There are many occasions in which the formulations of the invention are useful. For athletes, a transdermal delivery formulation can deliver to tired muscles sufficient amounts of a neutralizing agent for lactic acid, such as a ketone component, to relieve the burning sensation felt by the athlete due to the buildup of lactic acid. This permits the athlete to continue to perform at optimum level for longer periods of time. In addition, athletes or others “working out” are expending high amounts of energy and are in need of energy generation, especially in those areas of their musculature that are involved in performing workouts and, therefore, need to consume large numbers of calories. These nutrients can be supplied directly rather than requiring oral ingestion which is counterproductive and relatively slow.

The transdermal delivery formulations of the present disclosure may deliver medicaments of various sizes. As examples, the medicaments having a molecular weight of less than 500 Da, e.g., Nicotine (162.2 Da), Diphenhydramine Hydrochloride (291.8 Da), and Hydrocortisone (362.5 Da); medicaments having a molecular weight from 500 Da to 1000 Da, e.g., Sildenafil Citrate (666.7 Da), Neratinib (557 Da), and Doxycycline hyclate (512.9 Da); and medicaments having a molecular weight greater than 1000 Da, e.g., Cyclosporine A (1202.6 Da), Vancomycin Hydrochloride (1485.70 Da), and RBD protein (10,000+Da).

The medicament can be any of Polidocanol; Resveratrol; LUT014; MRTX1133; incobotulinum toxin A (XEOMIN®); or incobotulinum toxin B to incobotulinum toxin B.

Polidocanol (CAS No. 9002-92-0) is small molecule that relieves itching caused by eczema and dry skin and to treat varicose veins, hemangiomas, and vascular malformations.

MRTX1133 (CAS No.: 2621928-55-8) is a chemotherapeutic compound. MRTX1133 is a highly selective inhibitor of mutant KRAS G12D and can reversibly binds to the activated and inactivated KRAS G12D mutants and inhibit their activity. The specificity of MRTX1133 to KRAS G12D is more than 1000 times that of wild-type KRAS. MRTX1133 is a noncovalent, potent, and selective KRAS G12D inhibitor. MRTX1133 optimally fills the switch II pocket and extends three substituents to favorably interact with the protein, resulting in an estimated KD against KRAS G12D of 0.2 pM. MRTX1133 prevents SOS1-catalyzed nucleotide exchange and/or formation of the KRAS G12D/GTP/RAF1 complex, thereby inhibiting mutant KRAS-dependent signal transduction. MRTX1133 selectively inhibits KRAS G12D mutant, but not KRAS wild-type, tumor cells. MRTX1133 has single digit nanomolar activity in cellular assays and marked in vivo efficacy in tumor models harboring KRAS G12D mutations.

Incobotulinum toxin A (XEOMIN®) is a neurotoxin. That is useful for treating upper limb spasticity, cervical dystonia, blepharospasm, chronic sialorrhea, upper limb spasticity in, pediatrics (excluding spasticity caused by cerebral palsy), chronic sialorrhea in pediatrics, and excessive saliva, or drooling in patients with Parkinson's Disease (PD)/parkinsonism.

Resveratrol is a stilbenoid, a type of natural phenol, which may exhibit antioxidant properties, and may be produced by plants under certain conditions, for example, in response to pathogens, such as bacteria or fungi. Resveratrol may be incorporated into the formulations disclosed herein as an active pharmaceutical agent. Resveratrol may also prevent oxidation of the formulations herein, as well as serving as an active pharmaceutical agent. Resveratrol may be useful in reducing circulating free radicals, treating or reducing inflammation or an inflammatory response, treating or preventing cardiovascular disease, treating or preventing high blood pressure, treating or preventing cancer, treating or preventing metabolic syndrome, treating or preventing cognitive related disorders, treating or preventing Alzheimer's or dementia, increasing insulin sensitivity, treating or preventing diabetes, treating or preventing an appearance of aging skin, other uses, and combinations thereof.

LUT014 (6-methyl-5-N-[3-(7H-purin-6-yl)pyridin-2-yl]-1-N-[3-(trifluoromethoxy)phenyl]isoquinoline-1,5-diamine) is a small molecule synthetic organic compound, which is a B-Raf inhibitor. LUT014 may comprise an IC50 of 11.7 nM, and be used in treating a symptom of or preventing side effect of EGFRs, EGFR Inhibitor Induced Acneiform Lesions, and radiation dermatitis. EGFRs are used in the treatment of various cancers, including colorectal, melanoma, skin, lung, head & neck, urinary bladder, pancreatic and breast, cancers. Accordingly, LUT014 may also be used as part of a combination therapy in the treatment of cancers including colorectal, melanoma, skin, lung, head & neck, urinary bladder, pancreatic and breast, cancers, in treating a symptom of or preventing side effect of EGFRs, EGFR Inhibitor Induced Acneiform Lesions, and radiation dermatitis. LUT014 may also be used as part of a combination therapy in the treatment of cancers which are treated with EGFRs, or radiation therapy. LUT014 may also be used in aiding wound-healing, and in treating diabetic foot ulcers. LUT014 may also be used as a topical agent for treatment of cancers on or near the skin, such as melanoma, breast cancer, or prostate cancer. LUT014 may also be used in the treatment of cancers including colorectal, melanoma, skin, lung, head & neck, urinary bladder, pancreatic and breast cancers. Without being bound to a particular theory, LUT014 may counter the effect of B-Raf Inhibitors on wild type epithelial cells by overriding MAP Kinase pathway inhibition of EGFRs; by reversing the inhibitory effect of EGFR inhibitors on downstream proteins in the skin cells, LUT014 may reduce dose-limiting acneiform lesions associated EGFR Inhibitors treatment.

In embodiments, the medicament is a nucleic acid. Examples of nucleic acids useful in formulations of the present disclosure include a double-stranded DNA (dsDNA) molecule, a single-stranded DNA (ssDNA) molecule, a dsRNA molecule, a ssRNA molecule, a plasmid, an oligonucleotide, a synthetic RNA molecule, a miRNA molecule, an mRNA molecule, and an siRNA molecule. In some cases, the nucleic acid is an mRNA molecule, e.g., that encodes a polypeptide or protein.

Naporafenib (LXH254) is a type II ATP-competitive inhibitor that inhibits both B- and CRAF kinase activities at picomolar concentrations with a high degree of selectivity against a panel of 456 human kinases and in cell-based assays. In vitro, Naporafenib not only inhibits MAPK signaling activity in tumor models harboring BRAFV600 mutation, but also inhibits mutant N- and KRAS-driven signaling due to its ability to inhibit both RAF monomers and dimers with similar potencies. Naporafenib is orally bioavailable, demonstrates a direct PK/PD relationship and causes tumor regression in multiple cell line and primary human tumor derived xenograft models at well-tolerated doses. Upon administration, naporafenib binds to Raf proteins and inhibits Raf-mediated signal transduction pathways. This inhibits proliferation of Raf-overexpressing tumor cells. Raf protein kinases are critical enzymes in the Ras/Raf/MEK/ERK signaling pathway and are upregulated in a variety of cancer cell types. They play key roles in tumor cell proliferation and survival.

Throughout this disclosure, a medicament, agent, or other ingredient is described as being provided at specific ranges of doses, amounts, concentrations, percentages, units, volumes, and the like. For example, a medicament may be provided at range of doses of about 1 mg to about 10 mg. As used herein a range from about 1 mg to about 10 mg includes all dosages therebetween and any subranges therebetween. More specifically, the dosages may be about 1 mg, about 1.01 mg, about 1.02 mg, about 1.03 mg, about 1.04 mg, about 1.05 mg, about 1.06 mg, about 1.07 mg, about 1.08 mg, about 1.09 mg, about 1.1 mg, about 1.2 mg, about 1.3 mg, about 1.4 mg, about 1.5 mg, about 1.6 mg, about 1.7 mg, about 1.8 mg, about 1.9 mg, about 2 mg, about 2.1 mg, about 2.2 mg, about 2.3 mg, about 2.4 mg, about 2.5 mg, about 2.6 mg, about 2.7 mg, about 2.8 mg, about 2.9 mg, about 3 mg, about 3.1 mg, about 3.2 mg, about 3.3 mg, about 3.4 mg, about 3.5 mg, about 3.6 mg, about 3.7 mg, about 3.8 mg, about 3.9 mg, about 4 mg, about 4.1 mg, about 4.2 mg, about 4.3 mg, about 4.4 mg, about 4.5 mg, about 4.6 mg, about 4.7 mg, about 4.8 mg, about 4.9 mg, about 5 mg, about 5.1 mg, about 5.2 mg, about 5.3 mg, about 5.4 mg, about 5.5 mg, about 5.6 mg, about 5.7 mg, about 5.8 mg, about 5.9 mg, about 6 mg, about 6.1 mg, about 6.2 mg, about 6.3 mg, about 6.4 mg, about 6.5 mg, about 6.6 mg, about 6.7 mg, about 6.8 mg, about 6.9 mg, about 7 mg, about 7.1 mg, about 7.2 mg, about 7.3 mg, about 7.4 mg, about 7.5 mg, about 7.6 mg, about 7.7 mg, about 7.8 mg, about 7.9 mg, about 8 mg, about 8.1 mg, about 8.2 mg, about 8.3 mg, about 8.4 mg, about 8.5 mg, about 8.6 mg, about 8.7 mg, about 8.8 mg, about 8.9 mg, about 9 mg, about 9.1 mg, about 9.2 mg, about 9.3 mg, about 9.4 mg, about 9.5 mg, about 9.6 mg, about 9.7 mg, about 9.8 mg, about 9.9 mg, about 10 mg, and any dosage therebetween. Moreover, the ranges may be from about 1 mg to about 2 mg, from about 1 mg to about 3 mg, from about 1 mg to about 5 mg, from about 1 mg to about 7 mg, from about 1 mg to about 8 mg, from about 2 mg to about 3 mg, from about 2 mg to about 4 mg, from about 2 mg to about 6 mg, from about 2 mg to about 8 mg, from about 2 mg to about 9 mg, from about 2 mg to about 5 mg, from about 2 mg to about 7 mg, from about 3 mg to about 4 mg, from about 3 mg to about 5 mg, from about 3 mg to about 7 mg, from about 3 mg to about 9 mg, from about 3 mg to about 10 mg, from about 3 mg to about 6 mg, from about 3 mg to about 8 mg, from about 4 mg to about 5 mg, from about 4 mg to about 6 mg, from about 4 mg to about 8 mg, from about 4 mg to about 10 mg, from about 4 mg to about 7 mg, from about 4 mg to about 9 mg, from about 5 mg to about 6 mg, from about 5 mg to about 7 mg, from about 5 mg to about 9 mg, from about 5 mg to about 8 mg, from about 5 mg to about 10 mg, from about 6 mg to about 7 mg, from about 6 mg to about 8 mg, from about 6 mg to about 10 mg, from about 6 mg to about 9 mg, from about 7 mg to about 8 mg, from about 7 mg to about 9 mg, from about 7 mg to about 10 mg, from about 8 mg to about 9 mg, from about 8 mg to about 10 mg, or from about 9 mg to about 10 mg, and any subrange therebetween.

The amount of a medicament in a transdermal delivery formulation disclosed herein may comprise a percentage of a formulation or solution from about 0.00001% to about 0.5% (w/w) or (w/v), e.g., about 0.00005%, 0.00006%, 0.00007%, 0.00008%, 0.00009%, 0.0001%, 0.0002%, 0.0003%, 0.0004%, 0.0005%, 0.0006%, 0.0007%, 0.0008%, 0.0009%, 0.001%, 0.002%, 0.003%, 0.004%, 0.005%, 0.006%, 0.007%, 0.008%, 0.009%, 0.1%, 0.2%, 0.3%, 0.4%, or 0.5% or any (w/w) or (w/v) therebetween. The medicaments and their various dose ranges listed in Table 1 herein could be used in an above-mentioned dose or any dose from about 0.001% to about 30%. As examples, the various doses listed above would be applicable to any one of Incobotulinum toxin A to Incobotulinum toxin G or to a nucleic acid or to the one or more buffering agents disclosed herein.

The amount of a medicament in a transdermal delivery formulation disclosed herein may comprise a percentage of a formulation or solution from about 0.0001% to about 30% (w/w) or (w/v), e.g., about 0.0005%, 0.0006%, 0.0007%, 0.0008%, 0.0009%, 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 11%, 32%, 33%, 34%, 35%, 36%, 37%, 38%, 39%, or 40%, or any (w/w) or (w/v) therebetween. The medicaments and their various dose ranges listed in Table 1 herein could be used in an above-mentioned dose or any dose from about 0.001% to about 40%. As examples, the various doses listed above would be applicable to Polidocanol, Resveratrol, LUT014, MRTX1133, a nucleic acid, or to the one or more buffering agents disclosed herein.

The effective dose of a medicament and/or active agent may be an amount from about 0.0001 mg to about 10 mg, e.g., about 0.0001 mg, 0.0002 mg, 0.0003 mg, 0.0004 mg, 0.0005 mg, 0.0006 mg, 0.0007 mg, 0.0008 mg, 0.0009 mg, 0.001 mg, 0.002 mg, 0.003 mg, 0.004 mg, 0.005 mg, 0.006 mg, 0.007 mg, 0.008 mg, 0.009 mg, 0.01 mg, 0.015 mg, 0.02 mg, 0.025 mg, 0.03 mg, 0.035 mg, 0.04 mg, 0.045 mg, 0.05 mg, 0.055 mg, 0.06 mg, 0.065 mg, 0.07 mg, 0.075 mg, 0.08 mg, 0.085 mg, 0.09 mg, 0.095 mg, 0.1 mg, 0.15 mg, 0.2 mg, 0.25 mg, 0.3 mg, 0.35 mg, 0.4 mg, 0.45 mg, 0.5 mg, 0.55 mg, 0.6 mg, 0.65 mg, 0.7 mg, 0.75 mg, 0.8 mg, 0.85 mg, 0.9 mg, 0.95 mg, 1 mg, 1.1 mg, 1.2 mg, 1.3 mg, 1.4 mg, 1.5 mg, 1.6 mg, 1.7 mg, 1.8 mg, 1.9 mg, 2 mg, 2.1 mg, 2.2 mg, 2.3 mg, 2.4 mg, 2.5 mg, 2.6 mg, 2.7 mg, 2.8 mg, 2.9 mg, 3 mg, 3.1 mg, 3.2 mg, 3.3 mg, 3.4 mg, 3.5 mg, 3.6 mg, 3.7 mg, 3.8 mg, 3.9 mg, 4 mg, 4.1 mg, 4.2 mg, 4.3 mg, 4.4 mg, 4.5 mg, 4.6 mg, 4.7 mg, 4.8 mg, 4.9 mg, 5 mg, 5.1 mg, 5.2 mg, 5.3 mg, 5.4 mg, 5.5 mg, 5.6 mg, 5.7 mg, 5.8 mg, 5.9 mg, 6 mg, 6.1 mg, 6.2 mg, 6.3 mg, 6.4 mg, 6.5 mg, 6.6 mg, 6.7 mg, 6.8 mg, 6.9 mg, 7 mg, 7.1 mg, 7.2 mg, 7.3 mg, 7.4 mg, 7.5 mg, 7.6 mg, 7.7 mg, 7.8 mg, 7.9 mg, 8 mg, 8.1 mg, 8.2 mg, 8.3 mg, 8.4 mg, 8.5 mg, 8.6 mg, 8.7 mg, 8.8 mg, 8.9 mg, 9 mg, 9.1 mg, 9.2 mg, 9.3 mg, 9.4 mg, 9.5 mg, 9.6 mg, 9.7 mg, 9.8 mg, 9.9 mg, or 10 mg, or any dose therebetween. Various drugs and their dose ranges listed in Table 1 could be used in an above-mentioned dose. As examples, the various doses listed above would be applicable to Polidocanol or to a nucleic acid or to the one or more buffering agents disclosed herein.

The effective dose of a medicament and/or active agent may be an amount from about 10 mg to about 1000 mg, e.g., about 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, 500 mg, 520 mg, 540 mg, 560 mg, 580 mg, 600 mg, 620 mg, 640 mg, 660 mg, 680 mg, 700 mg, 720 mg, 740 mg, 760 mg, 780 mg, 800 mg, 820 mg, 840 mg, 860 mg, 880 mg, 900 mg, 920 mg, 940 mg, 960 mg, 980 mg, or 1000 mg, or any dose therebetween. Various drugs and their dose ranges listed in Table 1 could be used in an above-mentioned dose. As examples, the various doses listed above would be applicable to Polidocanol, Resveratrol, LUT014, MRTX1133 or Naporafenib or to the one or more buffering agents disclosed herein.

The effective dose of a medicament and/or agent may be an amount from about 0.1 ng/ml to about 10 ng/ml, e.g., about 0.1 ng/ml, 0.2 ng/ml, 0.3 ng/ml, 0.4 ng/ml, 0.5 ng/ml, 0.6 ng/ml, 0.7 ng/ml, 0.8 ng/ml, 0.9 ng/ml, 1 ng/ml, 1.1 ng/ml, 1.2 ng/ml, 1.3 ng/ml, 1.4 ng/ml, 1.5 ng/ml, 1.6 ng/ml, 1.7 ng/ml, 1.8 ng/ml, 1.9 ng/ml, 2 ng/ml, 2.1 ng/ml, 2.2 ng/ml, 2.3 ng/ml, 2.4 ng/ml, 2.5 ng/ml, 2.6 ng/ml, 2.7 ng/ml, 2.8 ng/ml, 2.9 ng/ml, 3 ng/ml, 3.1 ng/ml, 3.2 ng/ml, 3.3 ng/ml, 3.4 ng/ml, 3.5 ng/ml, 3.6 ng/ml, 3.7 ng/ml, 3.8 ng/ml, 3.9 ng/ml, 4 ng/ml, 4.1 ng/ml, 4.2 ng/ml, 4.3 ng/ml, 4.4 ng/ml, 4.5 ng/ml, 4.6 ng/ml, 4.7 ng/ml, 4.8 ng/ml, 4.9 ng/ml, 5 ng/ml, 5.1 ng/ml, 5.2 ng/ml, 5.3 ng/ml, 5.4 ng/ml, 5.5 ng/ml, 5.6 ng/ml, 5.7 ng/ml, 5.8 ng/ml, 5.9 ng/ml, 6 ng/ml, 6.1 ng/ml, 6.2 ng/ml, 6.3 ng/ml, 6.4 ng/ml, 6.5 ng/ml, 6.6 ng/ml, 6.7 ng/ml, 6.8 ng/ml, 6.9 ng/ml, 7 ng/ml, 7.1 ng/ml, 7.2 ng/ml, 7.3 ng/ml, 7.4 ng/ml, 7.5 ng/ml, 7.6 ng/ml, 7.7 ng/ml, 7.8 ng/ml, 7.9 ng/ml, 8 ng/ml, 8.1 ng/ml, 8.2 ng/ml, 8.3 ng/ml, 8.4 ng/ml, 8.5 ng/ml, 8.6 ng/ml, 8.7 ng/ml, 8.8 ng/ml, 8.9 ng/ml, 9 ng/ml, 9.1 ng/ml, 9.2 ng/ml, 9.3 ng/ml, 9.4 ng/ml, 9.5 ng/ml, 9.6 ng/ml, 9.7 ng/ml, 9.8 ng/ml, 9.9 ng/ml, or 10 ng/ml, or any dose therebetween. Various drugs and their dose ranges listed in Table 1 could be used in an above-mentioned dose. As examples, the various doses listed above would be applicable to any one of Incobotulinum toxin A to Incobotulinum toxin G or a nucleic acid.

The effective dose of a medicament and/or agent may be an amount from about 10 ng/ml to about 1000 ng/ml, e.g., about 10 ng/ml, 20 ng/ml, 30 ng/ml, 40 ng/ml, 50 ng/ml, 60 ng/ml, 70 ng/ml, 80 ng/ml, 90 ng/ml, 100 ng/ml, 110 ng/ml, 120 ng/ml, 130 ng/ml, 140 ng/ml, 150 ng/ml, 160 ng/ml, 170 ng/ml, 180 ng/ml, 190 ng/ml, 200 ng/ml, 210 ng/ml, 220 ng/ml, 230 ng/ml, 240 ng/ml, 250 ng/ml, 260 ng/ml, 270 ng/ml, 280 ng/ml, 290 ng/ml, 300 ng/ml, 310 ng/ml, 320 ng/ml, 330 ng/ml, 340 ng/ml, 350 ng/ml, 360 ng/ml, 370 ng/ml, 380 ng/ml, 390 ng/ml, 400 ng/ml, 410 ng/ml, 420 ng/ml, 430 ng/ml, 440 ng/ml, 450 ng/ml, 460 ng/ml, 470 ng/ml, 480 ng/ml, 490 ng/ml, 500 ng/ml, 510 ng/ml, 520 ng/ml, 530 ng/ml, 540 ng/ml, 550 ng/ml, 560 ng/ml, 570 ng/ml, 580 ng/ml, 590 ng/ml, 600 ng/ml, 610 ng/ml, 620 ng/ml, 630 ng/ml, 640 ng/ml, 650 ng/ml, 660 ng/ml, 670 ng/ml, 680 ng/ml, 690 ng/ml, 700 ng/ml, 710 ng/ml, 720 ng/ml, 730 ng/ml, 740 ng/ml, 750 ng/ml, 760 ng/ml, 770 ng/ml, 780 ng/ml, 790 ng/ml, 80 ng/ml, 810 ng/ml, 820 ng/ml, 830 ng/ml, 840 ng/ml, 850 ng/ml, 860 ng/ml, 870 ng/ml, 880 ng/ml, 890 ng/ml, 900 ng/ml, 910 ng/ml, 920 ng/ml, 930 ng/ml, 940 ng/ml, 950 ng/ml, 960 ng/ml, 970 ng/ml, 980 ng/ml, 990 ng/ml, or 1000 ng/ml, or any dose therebetween. Various drugs and their dose ranges listed in Table 1 could be used in an above-mentioned dose. As examples, the various doses listed above would be applicable to any one of Incobotulinum toxin A to Incobotulinum toxin G or a nucleic acid.

Table 1 list agents that can be administered transdermally according to some embodiments. The “% solution” indicates the relative dosing which is the amount in the transdermal solution.

TABLE 1

| | Medicament | Effective Dose | % Solution (Relative Dosing) |
|---|---|---|---|
| 1 | Polidocanol | 2.5 mg-15 mg | 0.25%-1.5% |
| 2 | MRTX1133 | 60 mg-900 mg or 120 mg and 600 mg | 6%-30% |
| 3 | Incobotulinum toxin A | 0.72 ng/mL-800 ng/mL | 0.000072%-0.08% |
| 4 | Incobotulinum toxin B | 0.72 ng/ml-800 ng/mL | 0.000072%-0.08% |
| 5 | Incobotulinum toxin C | 0.72 ng/mL-800 ng/mL | 0.000072%-0.08% |
| 6 | Incobotulinum toxin D | 0.72 ng/mL-800 ng/mL | 0.000072%-0.08% |
| 7 | Incobotulinum toxin E | 0.72 ng/mL-800 ng/mL | 0.000072%-0.08% |
| 8 | Incobotulinum toxin F | 0.72 ng/mL-800 ng/mL | 0.000072%-0.08% |
| 9 | Incobotulinum toxin G | 0.72 ng/mL-800 ng/mL | 0.000072%-0.08% |
| 10 | Resveratrol | 100-500 mg | 15%-20% |
| 11 | LUT014 | 1-500 mg | 1%-20% |
| 12 | Nucleic acid, e.g., RNA | 1 ng to 1 mg | 0.0001%-1% |
| 13 | Naporafenib | 200-600 mg | 20-30% |

### Methods of Treating a Disease or Disorder

Another aspect of the present disclosure is a method for treating a disease or disorder or reducing a symptom thereof. The method comprising steps of administering to a subject in need thereof any herein-disclosed transdermal delivery formulation and administering to the subject in need thereof a composition comprising one or more medicaments selected from Table 1 or comprising buffering agents as disclosed herein.

Methods of Treating Localized and/or Superficial Diseases and Disorders

Another aspect of the present disclosure is a method for treating a localized and/or superficial disease or disorder or reducing a symptom thereof. The method comprising steps of administering to a subject in need thereof any herein-disclosed transdermal delivery formulation and administering to the subject in need thereof a composition comprising one or more medicaments selected from Table 1 or comprising buffering agents as disclosed herein.

In embodiments of the method, the disease or disorder is ankylosing spondylitis, gout, dermatomyositis, juvenile idiopathic arthritis, atopic dermatitis, psoriasis, seborrheic dermatitis, urticaria, hidradenitis suppurativa, folliculitis, neuritis, otitis externa, otitis media, capillaritis, bursitis, tendinitis, chondritis, sinusitis, rhinitis, pharyngitis, laryngitis, tracheitis, bronchitis, pleuritis, mediastinitis, cellulitis, synovitis, myositis, enthesitis, fasciitis, capsulitis, epicondylitis, panniculitis, osteomyelitis, spondylitis, periostitis, vaginitis, vulvitis, mastitis, orchitis, sudden onset hearing loss, pruritus, seborrhea, nasal polyps, contact dermatitis, neurodermatitis, prurigo, bullous pemphigoid, keloids, hypertrophic scars, palmoplantar pustulosis, erythema multiforme, Duhring's disease, epidermolysis bullosa, or a combination thereof. In some embodiments of the method, the disease or disorders described herein may be treated with the medicament described herein. In some embodiments of the method, the disease or disorders described herein may be treated with the medicament selected from Table 1 or with a medicament that comprises one or more buffering agents described herein.

### Methods of Treating Local, Non-Superficial Diseases and Disorders

Another aspect of the present disclosure is a method for treating a localized, non-superficial disease or disorder or reducing a symptom thereof. The method comprising steps of administering to a subject in need thereof any herein-disclosed transdermal delivery formulation and administering to the subject in need thereof a composition comprising one or more medicaments selected from Table 1 or with a medicament that comprises one or more buffering agents as disclosed herein.

In embodiments of the method, the disease or disorder is endometriosis, asthma, autoimmune encephalitis, osteomyelitis, lupus, scleroderma, vasculitis, myelitis, myositis, Sjogren's syndrome, ulcerative colitis, familial mediterranean fever, neonatal onset multisystem inflammatory disease, tumor necrosis factor receptor-associated periodic syndrome, Behcet's disease, glomerulonephritis, hepatitis, endocarditis, myocarditis, pericarditis, appendicitis, Crohn's disease, amyloidosis, Still's disease, pelvic inflammatory disease, arteritis, gastritis, pancreatitis, peritonitis, cholecystitis, ureteritis, cystitis, urethritis, oophoritis, salpingitis, parametritis, cervicitis, prostatitis, hypophysitis, thyroiditis, parathyroiditis, adrenalitis, lymphangitis, lymphadenitis, rheumatoid arthritis, psoriatic arthritis, reactive arthritis, allergic rhinitis, sarcoidosis, atherosclerosis, hypertension, left ventricular hypertropy or a combination thereof. In some embodiments of the method, the disease or disorders described herein may be treated with the medicament described herein. In some embodiments of the method, the disease or disorders described herein may be treated with the medicament of Table 1 or with a medicament that comprises one or more buffering agents as described herein.

Methods of Treating Systemic Diseases with an Inflammatory Aspect

Another aspect of the present disclosure is a method for treating a systemic disease or disorder with an inflammatory aspect or reducing a symptom thereof. The method comprising steps of administering to a subject in need thereof any herein-disclosed transdermal delivery formulation and administering to the subject in need thereof a composition comprising one or more medicaments selected from Table 1 or with a medicament that comprises one or more buffering agents as disclosed herein.

In embodiments of the method, the disease or disorder is fatty liver disease, NASH, diabetes mellitus, chronic kidney disease, inflammatory bowel disease, Kawasaki disease, systemic inflammatory response syndrome, multiple sclerosis, cardiovascular disease, Alzheimer's disease, Parkinson's disease, or a combination thereof. In some embodiments of the method, the disease or disorders described herein may be treated with the medicament described herein. In some embodiments of the method, the disease or disorders described herein may be treated with one or more medicaments selected from Table 1, or with a medicament that comprise one or more buffering agent as described herein.

Additionally, the method may further include administering an additional anti-inflammatory agent described herein before administering the transdermal delivery formulation. The method may also include administering the anti-inflammatory agent contemporary with (e.g., at the same time) as the transdermal delivery formulation. The method may further include administering the anti-inflammatory agent after the transdermal delivery formulation.

The anti-inflammatory agent may be a steroidal anti-inflammatory agent such as hydrocortisone, hydrocortisone acetate, cortisone acetate, tixocortol pivalate, prednisolone, methylprednisolone, prednisone, amcinonide, budesonide, desonide, fluocinolone acetonide, fluocinonide, halcinonide, triamcinolone acetonide, beclometasone, betamethasone, dexamethasone, fluocortolone, halometasone, mometasone, ciclesonide, cortisone acetate, hydrocortisone aceponate, hydrocortisone acetate, hydrocortisone buteprate, hydrocortisone butyrate, hydrocortisone valerate, prednicarbate, or tixocortol pivalate.

The anti-inflammatory agent may be a non-steroidal anti-inflammatory agent such as acetylsalicylic acid, diflunisal, salicylic acid and its salts, salsalate, Ibuprofen, Dexibuprofen, Naproxen, Fenoprofen, Ketoprofen, Dexketoprofen, Flurbiprofen, Oxaprozin, Loxoprofen, Pelubiprofen, Zaltoprofen, Indomethacin, Tolmetin, Sulindac, Etodolac, Ketorolac, Diclofenac, Aceclofenac, Bromfenac, Nabumetone, Piroxicam, Meloxicam, Tenoxicam, Droxicam, Lornoxicam, Isoxicam, Phenylbutazone, Mefenamic acid, Meclofenamic acid, Flufenamic acid, Tolfenamic acid, Celecoxib, Refocoxib, Valdecoxib, Parecoxib, Lumiracoxic, Etoricoxib, Firocoxib, Nimesulide, Clonixin, or Licofelone.

### RAS/MAPK Driven Tumors

Another aspect of the present disclosure is a method for treating a RAS/MAPK driven cancer. The method comprising steps of administering to a subject in need thereof any herein-disclosed transdermal delivery formulation and administering to the subject in need thereof a composition comprising one or more medicaments selected from Table 1 or comprising one or more buffering agents as disclosed herein. In some embodiments of the method, the composition comprises one or more medicaments, wherein one of the medicaments is naporafenib.

In some embodiments of the method, the disease or disorder is cancer. In some embodiments of the method, the disease or disorder is RAS/MAPK driven tumors. In some embodiments of the method, the RAS/MAPK driven tumor is melanoma, unresectable metastatic melanoma, solid tumors, non-small cell lunger cancer, metastatic BRAF V600 colorectal cancer, pancreatic cancer, colorectal cancer, non-small cell lung cancer, acute myeloid leukemia, melanoma, bladder, thyroid, seminoma, liver diseases or disorders, kidney diseases or disorders, myelodysplastic syndrome, acute myelogenous leukemia, melanoma, hairy cell, ovarian cancer, breast cancer, or a combination thereof.

In some embodiments of the method, the RAS/MAPK driven tumor is melanoma, unresectable metastatic melanoma, solid tumors, non-small cell lunger cancer, metastatic BRAF V600 colorectal cancer, or combinations thereof. In some embodiments of the method, the disease or disorders described herein may be treated with one or more medicaments of Table 1 as described herein or with a medicament that comprise one or more buffering agent as described herein. In some embodiments of the method, the disease or disorders described herein may be treated with a medicament, wherein the medicament is naporafenib.

In some embodiments of the method, the disease or disorder is RAS/MAPK driven tumors. In some embodiments of the method, the RAS/MAPK driven tumor is pancreatic cancer, colorectal cancer, non-small cell lung cancer, acute myeloid leukemia, melanoma, bladder, thyroid, seminoma, liver diseases or disorders, kidney diseases or disorders, myelodysplastic syndrome, acute myelogenous leukemia, melanoma, hairy cell, ovarian cancer, breast cancer, or a combination thereof. In some embodiments of the method, the disease or disorders described herein may be treated with one or more medicaments of Table 1 as described herein or with a medicament that comprise one or more buffering agent as described herein. In some embodiments of the method, the disease or disorders described herein may be treated with a medicament, wherein the medicament is naporafenib.

In some embodiments of the formulation for transdermal delivery of a medicament, the medicament comprises naporafenib. In some embodiments of the method, the formulation for transdermal delivery of a medicament comprises naporafenib. In some embodiments of the method, the formulation for transdermal delivery of a medicament comprises a buffering agent, and the method further comprises administering naporafenib via its normal route (e.g., intravenous injection or oral dosage). In some embodiments of the method, the formulation comprises a transdermal formulation comprising a buffering agent and naporafenib.

In some embodiments of the method, the disease or disorders described herein may be treated with a transdermal formulation comprising a buffering agent. In some embodiments of the method, the disease or disorders described herein may be treated with a transdermal formulation comprising a buffering agent and naporafenib. In some embodiments of the method, the disease or disorders described herein may be treated with a transdermal formulation comprising a buffering agent, wherein the method further comprises administering with a chemotherapeutic agent. In some embodiments of the method, the chemotherapeutic agent is administered before, contemporary with, or after the transdermal delivery formulation is administered. The administration or co-administration of one or more formulation or composition of the invention and one or more chemotherapeutic agents can be used for the treatment of tumors or cancer in an animal, subject or patient.

In some embodiments of the method, the chemotherapeutic agent is directed to RAS/MAPK driven tumors. Chemotherapeutic agents include, but are not limited to alkylating agents, antibodies and related agents with anti-tumor properties, anthracyclines, antimetabolites, antitumor antibiotics, aromatase inhibitors, cytoskeletal disruptors (e.g., taxanes), epothilones, histone deacetylace inhibitors, kinase inhibitors, nucleoside analogues, topoisomerase inhibitors, retinoids, vinca alkaloids and derivatives, and the like.

As an example, alkylating agents can be administered or co-administered with or as part of a formulation provided herein. Examples of an alkylating agents that can be co-administered include mechlorethamine, chlorambucil, ifosfamide, melphalan, busulfan, carmustine, lomustine, procarbazine, dacardazine, cisplatin, carboplatin, mitomycin C, cyclophosphamide, ifosfamide, thiotepa, and dacarbazine, and analogues thereof. See for example U.S. Pat. No. 3,046,301 describing the synthesis of chlorambucil, U.S. Pat. No. 3,732,340 describing the synthesis of ifosfamide, U.S. Pat. No. 3,018,302 for the synthesis of cyclophosphamide, U.S. Pat. No. 3,032,584 describing the synthesis of melphalan, and Braunwald et al., “Harrison's Principles of Internal Medicine,” 15th Ed., McGraw-Hill, New York, N.Y., pp. 536-544 (2001) for clinical aspects of cyclophosphamide, chlorambucil, melphalan, ifosfamide, procarbazine, hexamethylmelamine, cisplatin, and carboplatin. Examples of nucleoside analogs include, but are not limited to, fludarabine pentostatin, methotrexate, fluorouracil, fluorodeoxyuridine, CB3717, azacitidine, cytarabine, floxuridine, mercaptopurine, 6-thioguanine, cladribine, and analogues thereof.

In embodiments of the method, the transdermal delivery formulation is administered before, contemporary with, or after the composition is administered.

In some embodiments of the method, the amount of the one or more medicament is the effective dose of the medicament as described in Table 1 or a medicament that comprises buffering agents as disclosed herein.

In various embodiments of the method, the composition is administered by the standard route for the medicament, e.g., the standard route is oral, topical, enteral, parenteral, by intravenous injection or infusion, by intraperitoneal injection, by intramuscular injection, or by subcutaneous injection. The composition may be a liquid, a suspension, a gel, a geltab, a semisolid, a tablet, a sachet, a lozenge, a pill, or a capsule.

Any medicament from Table 1 or a medicament that comprises buffering agents as disclosed herein can be used in a transdermal delivery formulation as a co-therapy with a composition comprising any medicament from Table 1 or any medicament comprising buffering agents as disclosed herein. In some cases, the medicament in the formulation is the same as the medicament in the composition. In other cases, the medicament in the formulation is different from the medicament in the composition. In yet other cases, the medicament in the formulation is also the medicament in the composition and the composition further comprises another medicament from Table 1 or comprises buffering agents as disclosed herein. In further cases, the medicament in the composition is also the medicament in the formulation and the formulation further comprises another medicament from Table 1 or comprises buffering agents as disclosed herein.

A transdermal delivery formulation may comprise mixtures wherein the components interact synergistically and induce skin permeation enhancements better than that induced by the individual components. Synergies between chemicals can be exploited to design potent permeation enhancers that overcome the efficacy limitations of single enhancers. Several embodiments disclosed herein utilize one or more distinct permeation enhancers.

Emergency medical treatment of individuals requiring, for example, blood balancing agents including electrolytes and readily-metabolized nutrients, such as glucose, that would otherwise be administered intravenously can instead be non-invasively treated by massaging the formulation through the skin and thus permitting systemic delivery so that levels in the bloodstream are altered.

Embodiments include a transdermal lotion or cream for administration of medicaments to a subject. It is placed on the skin to deliver a specific dose of an agent through the skin. The agent can be delivered across the skin into a localized subdermal location. For example, a lotion can alleviate inflammation from an autoimmune response. The lotion or cream can be applied directly to the affected area. Alternatively, the agent can enter the circulation for systemic distribution.

In an alternative embodiment, an agent can be administered using a transdermal or medicated adhesive patch. To release an agent, a patch can utilize a porous membrane covering a reservoir of the agent. Alternatively, the agent can be embedded in layers of the adhesive that release the agent as they dissolve or melt.

An advantage of a transdermal drug delivery route over other types of delivery is that the formulation can provide a controlled release of the agent. Conventional transdermal delivery systems are generally ineffective for use with agents and medications that are large molecules and/or hydrophilic molecules.

There are other advantages to transdermal administration of medicaments. Proteins and peptides used, for example, in aging treatments can be degraded by the gastric acid and enzymes. Transdermal administration is not affected by stomach or digestive issues. Further, people can benefit from drugs that are absorbed slowly and regularly. With a transdermal delivery formulation, a medicament can be released in small quantities over a long period of time.

Other advantages are related to dosing. Large doses of agents can cause dose-dependent toxicity in many cases. For example, oral administration of vitamin A can result in hypervitaminosis A. The main problems associated with the vitamin A are its half-life, fast absorption (due to lipophilicity) and its toxicity (due to high loading and frequent dosing). Also, some drugs undergo first-pass metabolism, which prevents their delivery to the desired site of action. Furthermore, many hydrophilic or lipophilic drugs show either poor dissolution or poor absorption on oral administration. With a transdermal delivery formulation, the effective concentration of an agent can be applied at the desired site without painful delivery.

In one embodiment, medicaments are supplied via transdermal administration. There are many occasions in which the formulations of the invention are useful. For athletes, a transdermal delivery formulation can deliver to tired muscles sufficient amounts of a neutralizing agent for lactic acid, such as a ketone component, to relieve the burning sensation felt by the athlete due to the buildup of lactic acid. This permits the athlete to continue to perform at optimum level for longer periods of time. In addition, athletes or others “working out” are expending high amounts of energy and are in need of energy generation, especially in those areas of their musculature that are involved in performing workouts and, therefore, need to consume large numbers of calories. These nutrients can be supplied directly rather than requiring oral ingestion which is counterproductive and relatively slow.

Certain methods of a transdermal delivery formulation provided herein may be supplemented with methods described in greater detail in the inventor's related applications mentioned above, including PCT/US2022/012330, filed Jan. 13, 2022; PCT/US2020/038558, filed Jun. 18, 2020; PCT/US18/51250 filed Sep. 14, 2018; and PCT/US18/28017 filed Apr. 17, 2018. The contents of each of which is incorporated by reference in its entirety.

### Transdermal Delivery Formulation Components

Embodiments include a transdermal lotion or cream for administration of a medicament to a subject. It is placed on the skin to deliver a specific dose of an agent through the skin. The agent can be delivered across the skin into a localized subdermal location.

In an alternative embodiment, an agent can be administered using a transdermal or medicated adhesive patch. To release an agent, a patch can utilize a porous membrane covering a reservoir of the agent. Alternatively, the agent can be embedded in layers of the adhesive that release the agent as they dissolve or melt.

An advantage of a transdermal drug delivery route over other types of delivery is that the formulation can provide a controlled release of the agent. Conventional transdermal delivery systems are generally ineffective for use with agents and medications that are large molecules and/or hydrophilic molecules.

There are other advantages to transdermal administration of medicaments. Small molecules can be inactivated or degraded by the stomach or liver. Transdermal administration is not affected by stomach or digestive issues. Further, people can benefit from drugs that are absorbed slowly and regularly. With a transdermal delivery formulation, a medicament can be released in small quantities over a long period of time.

Other advantages are related to dosing. Large doses of agents can cause dose-dependent toxicity in many cases. For example, oral administration of vitamin A can result in hypervitaminosis A. The main problems associated with the vitamin A are its half-life, fast absorption (due to lipophilicity) and its toxicity (due to high loading and frequent dosing). Also, some drugs undergo first-pass metabolism, which prevents their delivery to the desired site of action. Furthermore, many hydrophilic or lipophilic drugs show either poor dissolution or poor absorption on oral administration. With a transdermal delivery formulation, the effective concentration of an agent can be applied at the desired site without painful delivery.

In an embodiment, a transdermal delivery formulation contains a phosphatide in a concentration of at least 5%, at least 10%, at least 15%, at least 20%, at least 25%, at least 30%, at least 35%, at least 40%, at least 45%, at least 50%, at least 55%, at least 60%, at least 65%, at least 70% or more w/w of the transdermal delivery formulation.

In various embodiments, a formulation lacks a natural (e.g., plant or animal derived) lecithin.

In an embodiment, a transdermal delivery formulation contains a sterol or benzyl alcohol in a concentration of at least 1%, at least 2%, at least 3%, at least 4%, at least 5%, at least 6%, at least 7%, at least 8%, at least 9%, at least 10%, at least 15%, at least 20%, at least 25%, at least 30% or more w/w of the transdermal delivery formulation.

In an embodiment, a transdermal delivery formulation contains a carbohydrate in a concentration of at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70% or more w/w of the transdermal delivery formulation.

In various embodiments, a transdermal delivery formulation contains no glucose.

In an embodiment, a transdermal delivery formulation contains water in a concentration of at least 0.1%, at least 0.2%, at least 0.3%, at least 0.4%, at least 0.5%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70% or more w/w of the transdermal delivery formulation.

In various embodiments, a formulation lacks a natural (e.g., plant or animal derived) lecithin.

In a further embodiment, a fatty acid is a saturated or an unsaturated fatty acid. In another embodiment, an unsaturated fatty acid is myristoleic acid, palmitoleic acid, sapienic acid, oleic acid, elaidic acid, vaccenic acid, linoleic acid, linoelaidic acid, α-Linolenic acid, arachidonic acid, eicosapentaenoic acid, erucic acid and/or docosahexaenoic acid. In an embodiment, a saturated fatty acid is caprylic acid, capric acid, lauric acid, myristic acid, palmitic acid, stearic acid, arachidic acid, behenic acid, lignoceric acid and/or cerotic acid. In another embodiment, the fatty acid is a dietary fat and include duct fat, lard, tallow, butter, coconut oil, cocoa butter, palm kernel oil, palm oil, cottonseed oil, wheat germ oil, soybean oil, olive oil, corn oil, sunflower oil, safflower oil, hemp oil and/or canola/rapeseed oil.

In some embodiments, carotenoids are excluded from the formulations disclosed.

In an embodiment, a transdermal delivery formulation comprises the components of Table 2:

TABLE 2
General Formulation

| Ingredient | Weight (%) |
|---|---|
| Phospholipid | 5-15% |
| Emollient/moisturizer | 10-20% |
| Fatty acid | 0.5-2% |
| Alcohol | 0.5-2% |
| Oil | 1-5% |
| Surfactant | 0.5-2% |
| Deionized Water | 50-80% |
| Medicament | 0.001%-35%. |
| Total | 100.00% |

In Table 2, where an ingredient has weight percent that ranges from 5 to 15%, as an example, that ingredient may be present in the formulation at any percentage (w/w or w/v) from about 5% to about 15%. The weight percentage may be about 5% to about 15%. The weight percentage may be about 5% to about 6%, about 5% to about 7%, about 5% to about 8%, about 5% to about 9%, about 5% to about 10%, about 5% to about 11%, about 5% to about 12%, about 5% to about 13%, about 5% to about 14%, about 5% to about 15%, about 6% to about 7%, about 6% to about 8%, about 6% to about 9%, about 6% to about 10%, about 6% to about 11%, about 6% to about 12%, about 6% to about 13%, about 6% to about 14%, about 6% to about 15%, about 7% to about 8%, about 7% to about 9%, about 7% to about 10%, about 7% to about 11%, about 7% to about 12%, about 7% to about 13%, about 7% to about 14%, about 7% to about 15%, about 8% to about 9%, about 8% to about 10%, about 8% to about 11%, about 8% to about 12%, about 8% to about 13%, about 8% to about 14%, about 8% to about 15%, about 9% to about 10%, about 9% to about 11%, about 9% to about 12%, about 9% to about 13%, about 9% to about 14%, about 9% to about 15%, about 10% to about 11%, about 10% to about 12%, about 10% to about 13%, about 10% to about 14%, about 10% to about 15%, about 11% to about 12%, about 11% to about 13%, about 11% to about 14%, about 11% to about 15%, about 12% to about 13%, about 12% to about 14%, about 12% to about 15%, about 13% to about 14%, about 13% to about 15%, or about 14% to about 15%. The weight percentage may be about 5%, about 6%, about 7%, about 8%, about 9%, about 10%, about 11%, about 12%, about 13%, about 14%, or about 15%. The weight percentage may be at least about 5%, about 6%, about 7%, about 8%, about 9%, about 10%, about 11%, about 12%, about 13%, or about 14%. The weight percentage may be at most about 6%, about 7%, about 8%, about 9%, about 10%, about 11%, about 12%, about 13%, about 14%, or about 15%. Moreover, the weight percentage may be about 5% to about 6%. The weight percentage may be about 5% to about 5.1%, about 5% to about 5.2%, about 5% to about 5.3%, about 5% to about 5.4%, about 5% to about 5.5%, about 5% to about 5.6%, about 5% to about 5.7%, about 5% to about 5.8%, about 5% to about 5.9%, about 5% to about 6%, about 5.1% to about 5.2%, about 5.1% to about 5.3%, about 5.1% to about 5.4%, about 5.1% to about 5.5%, about 5.1% to about 5.6%, about 5.1% to about 5.7%, about 5.1% to about 5.8%, about 5.1% to about 5.9%, about 5.1% to about 6%, about 5.2% to about 5.3%, about 5.2% to about 5.4%, about 5.2% to about 5.5%, about 5.2% to about 5.6%, about 5.2% to about 5.7%, about 5.2% to about 5.8%, about 5.2% to about 5.9%, about 5.2% to about 6%, about 5.3% to about 5.4%, about 5.3% to about 5.5%, about 5.3% to about 5.6%, about 5.3% to about 5.7%, about 5.3% to about 5.8%, about 5.3% to about 5.9%, about 5.3% to about 6%, about 5.4% to about 5.5%, about 5.4% to about 5.6%, about 5.4% to about 5.7%, about 5.4% to about 5.8%, about 5.4% to about 5.9%, about 5.4% to about 6%, about 5.5% to about 5.6%, about 5.5% to about 5.7%, about 5.5% to about 5.8%, about 5.5% to about 5.9%, about 5.5% to about 6%, about 5.6% to about 5.7%, about 5.6% to about 5.8%, about 5.6% to about 5.9%, about 5.6% to about 6%, about 5.7% to about 5.8%, about 5.7% to about 5.9%, about 5.7% to about 6%, about 5.8% to about 5.9%, about 5.8% to about 6%, or about 5.9% to about 6%. The weight percentage may be about 5%, about 5.1%, about 5.2%, about 5.3%, about 5.4%, about 5.5%, about 5.6%, about 5.7%, about 5.8%, about 5.9%, or about 6%. The weight percentage may be at least about 5%, about 5.1%, about 5.2%, about 5.3%, about 5.4%, about 5.5%, about 5.6%, about 5.7%, about 5.8%, or about 5.9%. The weight percentage may be at most about 5.1%, about 5.2%, about 5.3%, about 5.4%, about 5.5%, about 5.6%, about 5.7%, about 5.8%, about 5.9%, or about 6%. Further, the weight percentage may be about 5% to about 5.1%. The weight percentage may be about 5% to about 5.01%, about 5% to about 5.02%, about 5% to about 5.03%, about 5% to about 5.04%, about 5% to about 5.05%, about 5% to about 5.06%, about 5% to about 5.07%, about 5% to about 5.08%, about 5% to about 5.09%, about 5% to about 5.1%, about 5.01% to about 5.02%, about 5.01% to about 5.03%, about 5.01% to about 5.04%, about 5.01% to about 5.05%, about 5.01% to about 5.06%, about 5.01% to about 5.07%, about 5.01% to about 5.08%, about 5.01% to about 5.09%, about 5.01% to about 5.1%, about 5.02% to about 5.03%, about 5.02% to about 5.04%, about 5.02% to about 5.05%, about 5.02% to about 5.06%, about 5.02% to about 5.07%, about 5.02% to about 5.08%, about 5.02% to about 5.09%, about 5.02% to about 5.1%, about 5.03% to about 5.04%, about 5.03% to about 5.05%, about 5.03% to about 5.06%, about 5.03% to about 5.07%, about 5.03% to about 5.08%, about 5.03% to about 5.09%, about 5.03% to about 5.1%, about 5.04% to about 5.05%, about 5.04% to about 5.06%, about 5.04% to about 5.07%, about 5.04% to about 5.08%, about 5.04% to about 5.09%, about 5.04% to about 5.1%, about 5.05% to about 5.06%, about 5.05% to about 5.07%, about 5.05% to about 5.08%, about 5.05% to about 5.09%, about 5.05% to about 5.1%, about 5.06% to about 5.07%, about 5.06% to about 5.08%, about 5.06% to about 5.09%, about 5.06% to about 5.1%, about 5.07% to about 5.08%, about 5.07% to about 5.09%, about 5.07% to about 5.1%, about 5.08% to about 5.09%, about 5.08% to about 5.1%, or about 5.09% to about 5.1%. The weight percentage may be about 5%, about 5.01%, about 5.02%, about 5.03%, about 5.04%, about 5.05%, about 5.06%, about 5.07%, about 5.08%, about 5.09%, or about 5.1%. The weight percentage may be at least about 5%, about 5.01%, about 5.02%, about 5.03%, about 5.04%, about 5.05%, about 5.06%, about 5.07%, about 5.08%, or about 5.09%. The weight percentage may be at most about 5.01%, about 5.02%, about 5.03%, about 5.04%, about 5.05%, about 5.06%, about 5.07%, about 5.08%, about 5.09%, or about 5.1%.

The other ranges recited in Table 2 include similar ranges and subranges and values within ranges.

In some cases, the medicament is in an amount from about 0.001% to about 0.01% w/w of the formulation, in an amount from about 0.011% to about 0.1% w/w of the formulation, in an amount from about 0.11% to about 1.0% w/w of the formulation, in an amount from about 1% to about 10% w/w of the formulation, in an amount from about 11% to about 20% w/w of the formulation, or in an amount from about 21% to about 30% w/w of the formulation.

In various embodiments, a transdermal delivery formulation comprises the components of Table 3:

TABLE 3
General Formulation

| Ingredient | Weight (%) |
|---|---|
| Phospholipid | 3-15% |
| Fatty acid ester | 5-20% |
| Long-chain fatty acids | 0.1-10% |
| Water | 30-90% |
| Medicament | 0.001%-35%. |
| Optionally: | |
| viscosity-improving | 5-20% |
| agent | |
| penetration | 0.5-5% |
| enhancer | |
| emulsifier | 0.5-10% |
| Total | 100.00% |

In Table 3, where an ingredient has weight percent that ranges from 5 to 20% (e.g., for the Fatty acid ester and viscosity-improving agent), as an example, that ingredient may be present in the formulation at any percentage (w/w or w/v) from about 5% to about 20%. The weight percentage may be about 5% to about 20%. The weight percentage may be about 5% to about 6%, about 5% to about 7%, about 5% to about 8%, about 5% to about 9%, about 5% to about 10%, about 5% to about 11%, about 5% to about 12%, about 5% to about 13%, about 5% to about 14%, about 5% to about 15%, about 6% to about 7%, about 6% to about 8%, about 6% to about 9%, about 6% to about 10%, about 6% to about 11%, about 6% to about 12%, about 6% to about 13%, about 6% to about 14%, about 6% to about 15%, about 7% to about 8%, about 7% to about 9%, about 7% to about 10%, about 7% to about 11%, about 7% to about 12%, about 7% to about 13%, about 7% to about 14%, about 7% to about 15%, about 8% to about 9%, about 8% to about 10%, about 8% to about 11%, about 8% to about 12%, about 8% to about 13%, about 8% to about 14%, about 8% to about 15%, about 9% to about 10%, about 9% to about 11%, about 9% to about 12%, about 9% to about 13%, about 9% to about 14%, about 9% to about 15%, about 10% to about 11%, about 10% to about 12%, about 10% to about 13%, about 10% to about 14%, about 10% to about 15%, about 10% to about 16%, about 10% to about 17%, about 10% to about 18%, about 10% to about 19%, about 10% to about 20%, about 11% to about 12%, about 11% to about 13%, about 11% to about 14%, about 11% to about 15%, about 11% to about 16%, about 11% to about 17%, about 11% to about 18%, about 11% to about 19%, about 11% to about 20%, about 12% to about 13%, about 12% to about 14%, about 12% to about 15%, about 12% to about 16%, about 12% to about 17%, about 12% to about 18%, about 12% to about 19%, about 12% to about 20%, about 13% to about 14%, about 13% to about 15%, about 13% to about 16%, about 13% to about 17%, about 13% to about 18%, about 13% to about 19%, about 13% to about 20%, about 14% to about 15%, about 14% to about 16%, about 14% to about 17%, about 14% to about 18%, about 14% to about 19%, about 14% to about 20%, about 15% to about 16%, about 15% to about 17%, about 15% to about 18%, about 15% to about 19%, about 15% to about 20%, about 16% to about 17%, about 16% to about 18%, about 16% to about 19%, about 16% to about 20%, about 17% to about 18%, about 17% to about 19%, about 17% to about 20%, about 18% to about 19%, about 18% to about 20%, or about 19% to about 20%, and any range therebetween. The weight percentage may be about 5%, about 6%, about 7%, about 8%, about 9%, about 10%, about 11%, about 12%, about 13%, about 14%, about 15%, about 16%, about 17%, about 18%, about 19%, or about 20%. The weight percentage may be at least about 5%, about 6%, about 7%, about 8%, about 9%, about 10%, about 11%, about 12%, about 13%, or about 14%. The weight percentage may be at most about 6%, about 7%, about 8%, about 9%, about 10%, about 11%, about 12%, about 13%, about 14%, about 15%, about 16%, about 17%, about 18%, about 19%, or about 20%. Moreover, the weight percentage may be about 5% to about 6%. The weight percentage may be about 5% to about 5.1%, about 5% to about 5.2%, about 5% to about 5.3%, about 5% to about 5.4%, about 5% to about 5.5%, about 5% to about 5.6%, about 5% to about 5.7%, about 5% to about 5.8%, about 5% to about 5.9%, about 5% to about 6%, about 5.1% to about 5.2%, about 5.1% to about 5.3%, about 5.1% to about 5.4%, about 5.1% to about 5.5%, about 5.1% to about 5.6%, about 5.1% to about 5.7%, about 5.1% to about 5.8%, about 5.1% to about 5.9%, about 5.1% to about 6%, about 5.2% to about 5.3%, about 5.2% to about 5.4%, about 5.2% to about 5.5%, about 5.2% to about 5.6%, about 5.2% to about 5.7%, about 5.2% to about 5.8%, about 5.2% to about 5.9%, about 5.2% to about 6%, about 5.3% to about 5.4%, about 5.3% to about 5.5%, about 5.3% to about 5.6%, about 5.3% to about 5.7%, about 5.3% to about 5.8%, about 5.3% to about 5.9%, about 5.3% to about 6%, about 5.4% to about 5.5%, about 5.4% to about 5.6%, about 5.4% to about 5.7%, about 5.4% to about 5.8%, about 5.4% to about 5.9%, about 5.4% to about 6%, about 5.5% to about 5.6%, about 5.5% to about 5.7%, about 5.5% to about 5.8%, about 5.5% to about 5.9%, about 5.5% to about 6%, about 5.6% to about 5.7%, about 5.6% to about 5.8%, about 5.6% to about 5.9%, about 5.6% to about 6%, about 5.7% to about 5.8%, about 5.7% to about 5.9%, about 5.7% to about 6%, about 5.8% to about 5.9%, about 5.8% to about 6%, or about 5.9% to about 6%. The weight percentage may be about 5%, about 5.1%, about 5.2%, about 5.3%, about 5.4%, about 5.5%, about 5.6%, about 5.7%, about 5.8%, about 5.9%, or about 6%. The weight percentage may be at least about 5%, about 5.1%, about 5.2%, about 5.3%, about 5.4%, about 5.5%, about 5.6%, about 5.7%, about 5.8%, or about 5.9%. The weight percentage may be at most about 5.1%, about 5.2%, about 5.3%, about 5.4%, about 5.5%, about 5.6%, about 5.7%, about 5.8%, about 5.9%, or about 6%. Further, the weight percentage may be about 5% to about 5.1%. The weight percentage may be about 5% to about 5.01%, about 5% to about 5.02%, about 5% to about 5.03%, about 5% to about 5.04%, about 5% to about 5.05%, about 5% to about 5.06%, about 5% to about 5.07%, about 5% to about 5.08%, about 5% to about 5.09%, about 5% to about 5.1%, about 5.01% to about 5.02%, about 5.01% to about 5.03%, about 5.01% to about 5.04%, about 5.01% to about 5.05%, about 5.01% to about 5.06%, about 5.01% to about 5.07%, about 5.01% to about 5.08%, about 5.01% to about 5.09%, about 5.01% to about 5.1%, about 5.02% to about 5.03%, about 5.02% to about 5.04%, about 5.02% to about 5.05%, about 5.02% to about 5.06%, about 5.02% to about 5.07%, about 5.02% to about 5.08%, about 5.02% to about 5.09%, about 5.02% to about 5.1%, about 5.03% to about 5.04%, about 5.03% to about 5.05%, about 5.03% to about 5.06%, about 5.03% to about 5.07%, about 5.03% to about 5.08%, about 5.03% to about 5.09%, about 5.03% to about 5.1%, about 5.04% to about 5.05%, about 5.04% to about 5.06%, about 5.04% to about 5.07%, about 5.04% to about 5.08%, about 5.04% to about 5.09%, about 5.04% to about 5.1%, about 5.05% to about 5.06%, about 5.05% to about 5.07%, about 5.05% to about 5.08%, about 5.05% to about 5.09%, about 5.05% to about 5.1%, about 5.06% to about 5.07%, about 5.06% to about 5.08%, about 5.06% to about 5.09%, about 5.06% to about 5.1%, about 5.07% to about 5.08%, about 5.07% to about 5.09%, about 5.07% to about 5.1%, about 5.08% to about 5.09%, about 5.08% to about 5.1%, or about 5.09% to about 5.1%. The weight percentage may be about 5%, about 5.01%, about 5.02%, about 5.03%, about 5.04%, about 5.05%, about 5.06%, about 5.07%, about 5.08%, about 5.09%, or about 5.1%. The weight percentage may be at least about 5%, about 5.01%, about 5.02%, about 5.03%, about 5.04%, about 5.05%, about 5.06%, about 5.07%, about 5.08%, or about 5.09%. The weight percentage may be at most about 5.01%, about 5.02%, about 5.03%, about 5.04%, about 5.05%, about 5.06%, about 5.07%, about 5.08%, about 5.09%, or about 5.1%.

The other ranges recited in Table 3 (e.g., 3-15% for the phospholipids; 0.1-10% for the Long-chain fatty acids; 30-90% for the water; 0.05%-5% for the PDE5 inhibitor; 0.5-5% for the penetration enhancer; and 0.5-10% for the emulsifier) recited in the above table include similar ranges and subranges and values within ranges. The present disclosure contemplates all similar ranges and subranges and values within ranges for each ingredient included in a formulation.

In some cases, the medicament is in an amount from about 0.001% to about 0.01% w/w of the formulation, in an amount from about 0.011% to about 0.1% w/w of the formulation, in an amount from about 0.11% to about 1.0% w/w of the formulation, in an amount from about 1% to about 10% w/w of the formulation, in an amount from about 11% to about 20% w/w of the formulation, or in an amount from about 21% to about 30% w/w of the formulation. The present disclosure contemplates all similar ranges and subranges and values within ranges for the PDE5 inhibitor or inhibitors included in a formulation.

In another embodiment, a transdermal delivery formulation comprises the components of

TABLE 4
Medicament “A” Formulation

| Ingredient | Weight (%) |
|---|---|
| Phosphatidylcholine | 7.64% |
| Isopropyl Palmitate | 13.30% |
| Stearic Acid | 0.62% |
| Benzyl Alcohol | 1.39% |
| Carthamus Tinctorius | 2.93% |
| (safflower) oil | |
| Oleic Acid | 0.97% |
| Polyglyceryl-4 | 1.06% |
| Laurate | |
| Deionized Water | 60.84% |
| Active Agent | 2.00% |
| Poloxamer 407 | 9.25% |
| Total | 100.00% |

In another embodiment, a transdermal delivery formulation comprises the components of Table 4:

TABLE 5
Medicament “B” Formulation

| | Weight |
|---|---|
| Ingredient | (%) |
| Phosphatidylcholine | 7.66% |
| Isopropyl Palmitate | 13.34% |
| Benzyl Alcohol | 1.39% |
| Stearic Acid | 0.68% |
| Carthamus Tinctorius | 2.79% |
| (safflower) oil | |
| Polyglyceryl-4 | 1.07% |
| Laurate | |
| Oleic Acid | 1.06% |
| Deionized Water | 61.73% |
| Active Agent | 1.00% |
| Poloxamer 407 | 9.28% |
| Total | 100.00% |

In some cases for the Medicament “A” Formulation or Medicament “B” Formulation, rather than the medicament being in an amount or about 1% or about 2% w/w of the formulation, the amount of the medicament is less than about 1% and the amount of water is increased proportionally. Alternately, rather than the medicament being in an amount or about 1% or about 2% w/w of the formulation, the amount of the medicament is greater than about 2% and the amount of water is decreased proportionally.

In an aspect, the concentration of phosphatidylcholine in a transdermal delivery formulation is at least 10%, at least 15%, at least 20%, at least 25%, at least 28.75%, at least 30%, at least 35%, at least 40% or more. In an aspect, the concentration of phosphatidylcholine in a transdermal delivery formulation is not more than 10%, not more than 15%, not more than 20%, not more than 25%, not more than 28.75%, not more than 30%, not more than 35%, not more than 40% or more. In an aspect, the concentration of phosphatidylcholine in a transdermal delivery formulation is about 10%, about 15%, about 20%, about 25%, at least 28.75%, about 30%, about 35%, about 40% or more. In an aspect, the concentration of Phosphatidylcholine in a transdermal delivery formulation is from 10% to 40%, is from 15% to 35%, is from 20% to 30%, is from 25% to 30%, is from 28% to 29%.

In a further embodiment, the concentration of benzyl alcohol in a transdermal delivery formulation is at least 0.25%, at least 0.5%, at least 0.75%, at least 1%, at least 2%, at least 2.5%, at least 3%, at least 4%, at least 5% or more. In an embodiment, the concentration of Benzyl Alcohol in a transdermal delivery formulation is about 0.25%, about 0.5%, about 0.75%, about 1%, about 2%, about 2.5%, about 3%, about 4%, about 5% or more. In another embodiment, the concentration of Benzyl Alcohol in a transdermal delivery formulation is at from 0.25% to 5%; from 0.5% to 4%, from 0.75% to 3%, from 1% to 2.5% or from 0.5% to 2%. In a further embodiment, the concentration of Benzyl Alcohol in a transdermal delivery formulation is no more than 0.25%, no more than 0.5%, no more than 0.75%, no more than 1%, no more than 2%, no more than 2.5%, no more than 3%, no more than 4%, no more than 5%.

In an embodiment, the concentration of deionized water in a transdermal delivery formulation is at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, at least 5% or more. In an embodiment, the concentration of Deionized Water in a transdermal delivery formulation is about 0.1%, about 0.2%, about 0.3%, about 0.4%, about 0.5%, about 0.6%, about 0.7%, about 0.8%, about 0.9%, about 1%, about 2%, about 3%, about 4%, about 5% or more. In an embodiment, the concentration of deionized water in a transdermal delivery formulation is from 0.1% to 5%, from 0.2% to 4%, from 0.3% to 3%, 0.4%-2%, 0.5% to 1%, from 0.6% t 0.9%, from 0.7% to 0.8%, from 0.4% to 1.5%, from 0.3% to 0.7% or from 0.4% to 0.6%. In an embodiment, the concentration of deionized water in a transdermal delivery formulation is no more than 0.1%, no more than 0.2%, no more than 0.3%, no more than 0.4%, no more than 0.5%, no more than 0.6%, no more than 0.7%, no more than 0.8%, no more than 0.9%, no more than 1%, no more than 2%, no more than 3%, no more than 4%, no more than 5% or more.

In an aspect, the concentration of safflower oil in a transdermal delivery formulation is at least 1%, at least 5%, at least 7.5%, at least 10%, at least 11%, at least 11.06%, at least 12%, at least 13%, at least 14%, at least 15%, at least 16%, at least 17%, at least 18%, at least 19%, at least 20% or more. In an aspect, the concentration of Safflower oil in a transdermal delivery formulation is about 1%, about 5%, about 7.5%, about 10%, about 11%, about 11.06%, about 12%, about 13%, about 14%, about 15%, about 16%, about 17%, about 18%, about 19%, about 20% or more. In an aspect, the concentration of Safflower oil in a transdermal delivery formulation is from 1% to 20%, from 5% to 19%, from 7.5% to 18%, from 10% to 17%, from 11% to 16%, from 11.06%, 12% from 11% to 12%, from 12% to 14%, from 13% to 14%, from 10% to 12%, from 10.5% to 12.5% or from 11% to 11.25%. In an aspect, the concentration of safflower oil in a transdermal delivery formulation is no more than 1%, no more than 5%, no more than 7.5%, no more than 10%, no more than 11%, no more than 11.06%, no more than 12%, no more than 13%, no more than 14%, no more than 15%, no more than 16%, no more than 17%, no more than 18%, no more than 19%, no more than 20%.

In a further aspect, the concentration of oleic acid in a transdermal delivery formulation is at least 1%, at least 2%, at least 3%, at least 3.65%, at least 4%, at least 5%, at least 6%, at least 7%, at least 8%, at least 9%, at least 10% or more. In a further aspect, the concentration of oleic acid in a transdermal delivery formulation is about 1%, about 2%, about 3%, about 3.65%, about 4%, about 5%, about 6%, about 7%, about 8%, about 9%, about 10% or more. In a further aspect, the concentration of Oleic Acid in a transdermal delivery formulation is no more than 1%, no more than 2%, no more than 3%, no more than 3.65%, no more than 4%, no more than 5%, no more than 6%, no more than 7%, no more than 8%, no more than 9%, no more than 10% or more. In another aspect, the concentration of Oleic Acid in a transdermal delivery formulation is from 1% to 10%, from 2% to 9%, from 2% to 3%, from 3% to 4%, from 3% to 8%, from 4% to 7%, from 5% to 6%, from 2 to 2.5% or from 2.5% to 4%.

In another aspect, the concentration of stearic acid in a transdermal delivery formulation is at least 1%, at least 2%, at least 2.34%, at least 3%, at least 4%, at least 5%, at least 6%, at least 7%, at least 8%, at least 9%, at least 10% or more. In another aspect, the concentration of stearic acid in a transdermal delivery formulation is no more than 1%, no more than 2%, no more than 2.34%, no more than 3%, no more than 4%, no more than 5%, no more than 6%, no more than 7%, no more than 8%, no more than 9%, no more than 10% or more. In another aspect, the concentration of stearic acid in a transdermal delivery formulation is about 1%, about 2%, about 2.34%, about 3%, about 4%, about 5%, about 6%, about 7%, about 8%, about 9%, about 10% or more. In another aspect, the concentration of stearic acid in a transdermal delivery formulation is from 1% to 10%, from 2% to 9%, from 2% to 3%, from 2.34% to 2.5%, from 3% to 8%, from 4% to 7%, from 5% to 6% or from 1.5% to 2.5%.

In an aspect, the concentration of isopropyl palmitate in a transdermal delivery formulation is at least 10%, at least 20%, at least 25%, at least 30%, at least 40%, at least 45%, at least 50%, at least 55%, at least 60%, at least 65%, at least 70%, at least 75% or more. In an aspect, the concentration of isopropyl palmitate in a transdermal delivery formulation is about 10%, about 20%, about 25%, about 30%, about 40%, about 45%, about 50%, about 55%, about 60%, about 65%, about 70%, about 75% or more. In an aspect, the concentration of isopropyl palmitate in a transdermal delivery formulation is no more than 10%, no more than 20%, no more than 25%, no more than 30%, no more than 40%, no more than 45%, no more than 50%, no more than 55%, no more than 60%, no more than 65%, no more than 70%, no more than 75% or more. In an aspect, the concentration of isopropyl palmitate in a transdermal delivery formulation is from 10% to 75%, from 20% to 70%, from 25% to 65%, from 30% to 60%, from 40% to 55%, from 45% to 50%, from 40% to 60%, from 45% to 55% or from 47% to 53%.

In an aspect, the concentration of poloxamer 407 in a transdermal delivery formulation is at least 10%, at least 15%, at least 20%, at least 25%, at least 28.75%, at least 30%, at least 35%, at least 40% or more. In an aspect, the concentration of poloxamer 407 in a transdermal delivery formulation is not more than 10%, not more than 15%, not more than 20%, not more than 25%, not more than 28.75%, not more than 30%, not more than 35%, not more than 40% or more. In an aspect, the concentration of poloxamer 407 in a transdermal delivery formulation is about 10%, about 15%, about 20%, about 25%, at least 28.75%, about 30%, about 35%, about 40% or more. In an aspect, the concentration of poloxamer 407 in a transdermal delivery formulation is from 10% to 40%, is from 15% to 35%, is from 20% to 30%, is from 25% to 30%, is from 28% to 29%.

In another aspect, the formulation includes glucose. The concentration of glucose in a transdermal delivery formulation can be, for example, at least 1%, at least 2%, at least 2.5%, at least 3%, at least 4%, at least 5%, at least 6%, at least 7%, at least 8%, at least 9% or more. In another aspect, the concentration of glucose in a transdermal delivery formulation is about 1%, about 2%, about 2.5%, about 3%, about 4%, about 5%, about 6%, about 7%, about 8%, about 9% or more. In another aspect, the concentration of glucose in a transdermal delivery formulation is no more than 1%, no more than 2%, no more than 2.5%, no more than 3%, no more than 4%, no more than 5%, no more than 6%, no more than 7%, no more than 8%, no more than 9% or more. In another aspect, the concentration of glucose in a transdermal delivery formulation is from 1% to 10%, is from 2% to 9%, is from 2.5% to 5%, is from 2% to 3%, is from 3% to 8%, if from 4% to 7%, if from 5% to 6%, is from 2% to 4%, is from 1.5% to 3.5%. In various embodiments, a transdermal delivery formulation contains no glucose.

A transdermal delivery formulation can include mixtures wherein the components interact synergistically and induce skin permeation enhancements better than that induced by the individual components. Synergies between chemicals can be exploited to design potent permeation enhancers that overcome the efficacy limitations of single enhancers. Several embodiments disclosed herein utilize three to five distinct permeation enhancers.

In some embodiments, a transdermal delivery formulation comprises phosphatidylcholine in amount less than 12% w/w or 18% w/w of the formulation. In some embodiments, the transdermal delivery formulation comprises a phospholipid in amount less than 12% w/w or 18% w/w of the formulation. In some embodiments, the transdermal delivery formulation comprises a mixture of tridecane and undecane in amount less than 2% w/w, 5% w/w, or 8% w/w of the formulation. In some embodiments, the formulation comprises Cetiol Ultimate® in an amount less than about 2% w/w, 5% w/w, or 10% w/w, or an equivalent mixture of tridecane and undecane. In some embodiments, the transdermal delivery formulation comprises cetyl alcohol in amount less than 2% w/w, 5% w/w, or 8% w/w of the formulation. In some embodiments, the transdermal delivery formulation comprises benzyl alcohol in an amount less than about 2% w/w, 5% w/w, or 8% w/w. In some embodiments, the transdermal delivery formulation comprises stearic acid in an amount less than 2% w/w, 5% w/w, or 8% w/w of the formulation.

For topical administration, and in particular transdermal administration, a transdermal delivery formulation will comprise penetrants including either or both chemical penetrants (CPEs) and peptide-based cellular penetrating agents (CPPs) that encourage transmission across the dermis and/or across membranes including cell membranes, as would be the case in particular for administration by suppository or intranasal administration, but for transdermal administration as well. In some embodiments, suitable penetrants include those that are described in the above-referenced US2009/0053290, WO2014/209910, WO2017/127834, and WO2022/155352. In addition to transdermal delivery formulations with penetrants, transdermal delivery can be affected by mechanically disrupting the surface of the skin to encourage penetration, or simply by supplying the formulation applied to the skin under an occlusive patch.

Alternatively, the transdermal delivery formulation comprises a completion component as well as one or more electrolytes sufficient to impart viscosity and viscoelasticity, one or more surfactants and an alcohol. The completion component can be a polar liquid, a non-polar liquid or an amphiphilic substance. The penetrant may further comprise a keratinolytic agent effective to reduce thiol linkages, disrupt hydrogen bonding and/or effect keratin lysis and/or a cell penetrating peptide (sometimes referred to as a skin-penetrating peptide) and/or a permeation enhancer.

Suitable gelling components also include isopropyl palmitate, ethyl laurate, ethyl myristate and isopropyl myristate. In some embodiments, a transdermal delivery formulation comprises a gelling agent in an amount less than 5% w/w of a transdermal delivery formulation. Certain hydrocarbons, such as cyclopentane, cyclooctane, trans-decalin, trans-pinane, n-pentane, n-hexane, n-hexadecane may also be used. In some embodiments, the transdermal delivery formulation comprises a mixture of xanthan gum, sclerotium gum, pullulan, or a combination thereof in an amount less than 2% w/w, 5% w/w, or 10% w/w of the formulation. In some embodiments, a transdermal delivery formulation comprises Siligel™ in an amount from about 1 to about 5% w/w or from about 5 to about 15% w/w, or an equivalent mixture of xanthan gum, sclerotium gum, and pullulan. In some embodiments, a transdermal delivery formulation comprises a mixture of caprylic triglycerides and capric triglycerides in amount less than 2% w/w, 8% w/w, or 10% w/w of the formulation. In some embodiments, a transdermal delivery formulation comprises Myritol® 312 in an amount from about 0.5 to about 10% w/w, or an equivalent mixture of caprylic triglycerides and capric triglycerides.

In some embodiments, a transdermal delivery formulation is in an amount from about 10 to about 90% w/w or from about 10 to about 50% w/w of the formulation or at least 10% w/w, at least 20% w/w, at least 30% w/w, at least 40% w/w, at least 50% w/w, at least 60% w/w, at least 70% w/w, at least 80% w/w, at least 90% w/w or at least 95% w/w. In some embodiments, a transdermal delivery formulation comprises phosphatidyl choline in amount less than 7% w/w, less than 8% w/w, less than 9% w/w, less than 10% w/w, less than 11% w/w, less than 12% w/w, less than 13% w/w, less than 14% w/w, less than 15% w/w, less than 16% w/w, less than 17% w/w or less than 18% w/w of the formulation. In some embodiments, a transdermal delivery formulation comprises a phospholipid in amount less than 20% w/w, less than 30% w/w, less than 40% w/w, less than or 50% w/w of the formulation. In some embodiments, a transdermal delivery formulation comprises a mixture of tridecane and undecane in amount less than 2% w/w, 3% w/w, 4% w/w, 5% w/w, 6% w/w, 7% w/w, or 8% w/w of the formulation. In some embodiments, the formulation comprises Cetiol Ultimate® in an amount less than about 2% w/w, 3% w/w, 4% w/w, 5% w/w, 6% w/w, 7% w/w, 8% w/w, 9% w/w, or 10% w/w, or an equivalent mixture of tridecane and undecane. In some embodiments, a transdermal delivery formulation comprises cetyl alcohol in amount less than 2% w/w, 3% w/w, 4% w/w, 5% w/w, 6% w/w, 7% w/w, 8% w/w, 9% w/w, or 10% w/w of the formulation. In some embodiments, the formulation comprises benzyl alcohol in an amount less than about 2% w/w, 3% w/w, 4% w/w, 5% w/w, 6% w/w, 7% w/w, 8% w/w, 9% w/w, or 10% w/w. In some embodiments, a transdermal delivery formulation comprises stearic acid in an amount less than 2% w/w, 3% w/w, 4% w/w, 5% w/w, 6% w/w, 7% w/w, 8% w/w, 9% w/w, or 10% w/w of the formulation. In some embodiments, the transdermal delivery formulation comprises phosphatidylcholine, hydrogenated phosphatidylcholine, phosphatidylserine, phosphatidylethanolamine, phosphatidylinositol, one or more phosphatides, one or more Inositol phosphatides, or combinations thereof, in amount less than 30% w/w or in amount less than 12% w/w of the formulation.

An additional component in a transdermal delivery formulation of the disclosure is an alcohol. Benzyl alcohol and ethanol are illustrated in the Examples. In particular, derivatives of benzyl alcohol which contain substituents on the benzene ring, such as halo, alkyl and the like. The weight percentage of benzyl or other related alcohol in the final composition is 0.5-20% w/w, and again, intervening percentages such as 1% w/w, 2% w/w, 3% w/w, 4% w/w, 5% w/w, 6% w/w, 7% w/w, 8% w/w, 9% w/w, or 10% w/w, and other intermediate weight percentages are included. Due to the aromatic group present in a transdermal delivery formulation such as benzyl alcohol, the molecule has a polar end (the alcohol end) and a non-polar end (the benzene end). This enables the agent to dissolve a wider variety of transdermal delivery formulation components.

In some embodiments, as noted above, the performance of a transdermal delivery formulation is further improved by including a nonionic detergent and polar gelling agent or including a powdered surfactant. In both aqueous and anhydrous forms of the composition, detergents, typically nonionic detergents are added. In general, the nonionic detergent should be present in an amount from about 1% w/w to about 30% w/w of a transdermal delivery formulation. Typically, in the compositions wherein a transdermal delivery formulation is topped off with a polar or aqueous solution containing detergent, the amount of detergent is relatively low—e.g., 2-25% w/w, or 5-15% w/w or 7-12% w/w of a transdermal delivery formulation. However, in compositions that are essentially anhydrous and are topped-off by powdered detergent, relatively higher percentages are usually used—e.g., 20-60% w/w.

In some embodiments, a transdermal delivery formulation further comprises a detergent portion in an amount from about 1 to about 70% w/w or from about 1 to about 60% w/w of a transdermal delivery formulation. In some embodiments, the nonionic detergent provides suitable handling properties whereby the formulations are gel-like or creams at room temperature. To exert this effect, the detergent, typically a poloxamer, is present in an amount from about 2 to about 12% w/w of a transdermal delivery formulation, preferably from about 5 to about 25% w/w in polar formulations. In the anhydrous forms of the compositions, the detergent is added in powdered or micronized form to bring the composition to 100% and higher amounts are used. In compositions with polar constituents, rather than bile salts, the nonionic detergent is added as a solution to bring the composition to 100%. If smaller amounts of detergent solutions are needed due to high levels of the remaining components, more concentrated solutions of the nonionic detergent are employed. Thus, for example, the percent detergent in the solution may be 10% to 40% or 20% or 30% and intermediate values depending on the percentages of the other components.

Suitable nonionic detergents include poloxamers such as the non-ionic surfactant Pluronic® and any other surfactant characterized by a combination of hydrophilic and hydrophobic moieties. Poloxamers are triblock copolymers of a central hydrophobic chain of polyoxypropylene flanked by two hydrophilic chains of polyethyleneoxide. Other nonionic surfactants include long chain alcohols and copolymers of hydrophilic and hydrophobic monomers where blocks of hydrophilic and hydrophobic portions are used.

In some embodiments, a transdermal delivery formulation also contains surfactant, typically, nonionic surfactant at 2-25% w/w of a transdermal delivery formulation along with a polar solvent wherein the polar solvent is present in an amount at least in molar excess of the nonionic surfactant. In these embodiments, typically, the composition comprises the above-referenced amounts of a transdermal delivery formulation and benzyl alcohol along with a sufficient amount of a polar solution, typically an aqueous solution or polyethylene glycol solution that itself contains 10%-40% of surfactant, typically nonionic surfactant to bring the composition to 100%.

Other examples of surfactants include polyoxyethylated castor oil derivatives such as HCO-60 surfactant sold by the HallStar Company; nonoxynol; octoxynol; phenylsulfonate; poloxamers such as those sold by BASF as Pluronic® F68, Pluronic® F127, and Pluronic® L62; polyoleates; Rewopal® HVIO, sodium laurate, sodium lauryl sulfate (sodium dodecyl sulfate); sodium oleate; sorbitan dilaurate; sorbitan dioleate; sorbitan monolaurate such as Span® 20 sold by Sigma-Aldrich; sorbitan monooleates; sorbitan trilaurate; sorbitan trioleate; sorbitan monopalmitate such as Span® 40 sold by Sigma-Aldrich; sorbitan stearate such as Span® 85 sold by Sigma-Aldrich; polyethylene glycol nonylphenyl ether such as Synperonic® NP sold by Sigma-Aldrich; p-(1,1,3,3-tetramethylbutyl)-phenyl ether sold as Triton™ X-100 sold by Sigma-Aldrich; and polysorbates such as polyoxyethylene (20) sorbitan monolaurate sold as Tween® 20, polysorbate 40 (polyoxyethylene (20) sorbitan monopalmitate) sold as Tween® 40, polysorbate 60 (polyoxyethylene (20) sorbitan monostearate) sold as Tween® 60, polysorbate 80 (polyoxyethylene (20) sorbitan monooleate) sold as Tween® 80, and polyoxyethylenesorbitan trioleate sold as Tween® 85 by Sigma-Aldrich. The weight percentage range of nonionic surfactant is in the range of 3% w/w−15% w/w, and again includes intermediate percentages such as 5% w/w, 7% w/w, 10% w/w, 12% w/w, and the like. In some embodiments, the detergent portion comprises a nonionic surfactant in an amount from about 1 to about 30% w/w of the formulation, and a polar solvent in an amount less than 5% w/w of the formulation. In some embodiments, the nonionic surfactant is a poloxamer and the polar solvent is water, an alcohol, or a combination thereof. In some embodiments, the detergent portion comprises poloxamer, propylene glycol, glycerin, ethanol, 50% w/v sodium hydroxide solution, or a combination thereof. In some embodiments, the detergent portion comprises glycerin in an amount less than 3% w/w of the formulation.

In the presence of a polar gelling agent, such as water, glycerol, ethylene glycol or formamide, a micellular structure is also often achieved. Typically, the polar agent is in molar excess of the nonionic detergent. The inclusion of the nonionic detergent/polar gelling agent combination results in a more viscous and cream-like or gel-like formulation which is suitable for application directly to the skin. This is typical of the aqueous forms of the composition.

In some embodiments other additives are included such as a gelling agent, a dispersing agent and a preservative. An example of a suitable gelling agent is hydroxypropylcellulose, which is generally available in grades from viscosities of from about 5 cps to about 25,000 cps such as about 1500 cps. All viscosity measurements are assumed to be made at room temperature unless otherwise stated. The concentration of hydroxypropylcellulose may range from about 1% w/w to about 2% w/w of the composition. Other gelling agents are known in the art and can be used in place of, or in addition to hydroxypropylcellulose. An example of a suitable dispersing agent is glycerin. Glycerin is typically included at a concentration from about 5% w/w to about 25% w/w of the composition. A preservative may be included at a concentration effective to inhibit microbial growth, ultraviolet light and/or oxygen-induced breakdown of composition components, and the like. When a preservative is included, it may range in concentration from about 0.01% w/w to about 1.5% w/w of the composition.

Additional components that can also be included in a transdermal delivery formulation are fatty acids, terpenes, lipids, and cationic, and anionic detergents. In some embodiments, a transdermal delivery formulation further comprises tranexamic acid in an amount less than 2% w/w, 5% w/w, or 10% w/w of the formulation. In some embodiments, a transdermal delivery formulation further comprises a polar solvent in an amount less than 2% w/w, 5% w/w, 10% w/w, or 20% w/w of the transdermal delivery formulation. In some embodiments, a transdermal delivery formulation further comprises a humectant, an emulsifier, an emollient, or a combination thereof. In some cases, the humectant is propylene glycol. In some cases, the emulsifier is polyglyceryl-4-laurate, cetyl alcohol or Durosoft PK-SG. In some cases, the emollient is derived from almond oil. In some embodiments, a transdermal delivery formulation further comprises almond oil in an amount less than about 5% w/w. In some embodiments, a formulation further comprises a mixture of thermoplastic polyurethane and polycarbonate in an amount less than about 5% w/w. In some embodiments, a transdermal delivery formulation further comprises phosphatidylethanolamine in an amount less than about 5% w/w. In some embodiments, a transdermal delivery formulation further comprises an inositol phosphatide in an amount less than about 5% w/w.

Other solvents and related compounds that can be used in some embodiments include acetamide and derivatives, acetone, n-alkanes (chain length from 7 to 16), alkanols, diols, short chain fatty acids, cyclohexyl-1,1-dimethylethanol, dimethyl acetamide, dimethyl formamide, ethanol, ethanol/d-limonene combination, 2-ethyl- 1,3-hexanediol, ethoxydiglycol (Transcutol® by Gattefosse, Lyon, France), glycerol, glycols, lauryl chloride, limonene N-methylformamide, 2-phenylethanol, 3-phenyl-1-propanol, 3-phenyl-2-propen-1-ol, polyethylene glycol, polyoxyethylene sorbitan monoesters, polypropylene glycol 425, primary alcohols (tridecanol), 1,2-propane diol, butanediol, C₃-C₆ triols or their mixtures and a polar lipid compound selected from C₁₆ or C₁₈ monounsaturated alcohol, C₁₆ or C₁₈ branched saturated alcohol and their mixtures, propylene glycol, sorbitan monolaurate sold as Span® 20 by Sigma-Aldrich, squalene, triacetin, trichloroethanol, trifluoroethanol, trimethylene glycol and xylene.

Fatty alcohols, fatty acids, fatty esters, are bilayer fluidizers that can be used in some embodiments. Examples of suitable fatty alcohols include aliphatic alcohols, decanol, lauryl alcohol (dodecanol), unolenyl alcohol, nerolidol, 1-nonanol, n-octanol, and oleyl alcohol. Examples of suitable fatty acid esters include butyl acetate, cetyl lactate, decyl N,N-dimethylamino acetate, decyl N,N-dimethylamino isopropionate, diethyleneglycol oleate, diethyl sebacate, diethyl succinate, diisopropyl sebacate, dodecyl N,N-dimethyamino acetate, dodecyl (N,N-dimethylamino)-butyrate, dodecyl N,N-dimethylamino isopropionate, dodecyl 2-(dimethyamino) propionate, E0-5-oleyl ether, ethyl acetate, ethylaceto acetate, ethyl propionate, glycerol monoethers, glycerol monolaurate, glycerol monooleate, glycerol monolinoleate, isopropyl isostearate, isopropyl linoleate, isopropyl myristate, isopropyl myristate/fatty acid monoglyceride combination, isopropyl palmitate, methyl acetate, methyl caprate, methyl laurate, methyl propionate, methyl valerate, 1-monocaproyl glycerol, monoglycerides (medium chain length), nicotinic esters (benzyl), octyl acetate, octyl N,N-dimethylamino acetate, oleyl oleate, n-pentyl N-acetylprolinate, propylene glycol monolaurate, sorbitan dilaurate, sorbitan dioleate, sorbitan monolaurate, sorbitan monooleate, sorbitan trilaurate, sorbitan trioleate, sucrose coconut fatty ester mixtures, sucrose monolaurate, sucrose monooleate, tetradecyl N,N-dimethylamino acetate. Examples of suitable fatty acid include alkanoic acids, caprid acid, diacid, ethyloctadecanoic acid, hexanoic acid, lactic acid, lauric acid, linoelaidic acid, linoleic acid, linolenic acid, neodecanoic acid, oleic acid, palmitic acid, pelargonic acid, propionic acid, and vaccenic acid. Examples of suitable fatty alcohol ethers include a-monoglyceryl ether, E0-2-oleyl ether, E0-5-oleyl ether, E0-10-oleyl ether, ether derivatives of polyglycerols and alcohols, and (1-O-dodecyl-3-O-methyl-2-O-(2′,3′-dihydroxypropyl glycerol).

Examples of completing agents that can be used in some embodiments include β- and γ-cyclodextrin complexes, hydroxypropyl methylcellulose (e.g., Carbopol® 934), liposomes, naphthalene diamide diimide, and naphthalene diester diimide.

One or more anti-oxidants can be included, such as vitamin C, vitamin E, proanthocyanidin and a-lipoic acid typically in concentrations of 0.1%-2.5% w/w.

In some applications, it is desirable to adjust the pH of a transdermal delivery formulation to assist in permeation or to adjust the nature of the target compounds in the subject. In some instances, the pH is adjusted to a level of pH 9-11 or 10-11 which can be done by providing appropriate buffers or simply adjusting the pH with base.

A transdermal delivery formulation can include other components that act as excipients or serve purposes other than for muscle performance and recovery. For example, preservatives like antioxidants e.g., ascorbic acid or α-lipoic acid and antibacterial agents may be included. Other components apart from therapeutically active ingredients and components that are the primary effectors of dermal penetration may include those provided for aesthetic purposes such as menthol or other aromatics, and components that affect the physical state of the composition such as emulsifiers, for example, Durosoft®. Typically, these ingredients are present in very small percentages of the compositions. It is understood that these latter ancillary agents are neither therapeutically ingredients nor are they components that are primarily responsible for penetration of the skin. The components that primarily effect skin penetration have been detailed as described above. However, some of these substances have some capability for effecting skin penetration. See, for example, Kunta, J. R. et al, J. Pharm. Sci. (1997) 86:1369-1373, describing penetration properties of menthol.

The application method is determined by the nature of the treatment but may be less critical than the nature of the formulation itself. If the application is to a skin area, it may be helpful in some instances to prepare the skin by cleansing or exfoliation. In some instances, it is helpful to adjust the pH of the skin area prior to application of a transdermal delivery formulation itself. The application of a transdermal delivery formulation may be by simple massaging onto the skin or by use of devices such as syringes or pumps. Patches could also be used. In some cases, it is helpful to cover the area of application to prevent evaporation or loss of a transdermal delivery formulation.

Where the application area is essentially skin, it is helpful to seal-off the area of application subsequent to supplying a transdermal delivery formulation and allowing the penetration to occur so as to restore the skin barrier. A convenient way to do this is to apply a composition comprising linoleic acid which effectively closes the entrance pathways that were provided by the penetrants of the invention. This application, too, is done by straightforward smearing onto the skin area or can be applied more precisely in measured amounts.

In addition to the compositions and formulations of the invention per se, the methods can employ a subsequent treatment with linoleic acid. As transdermal treatments generally open up the skin barrier, which is, indeed, their purpose, it is useful to seal the area of application after the treatment is finished. Thus, treatment with a transdermal delivery formulation may be followed by treating the skin area with a composition comprising linoleic acid to seal off the area of application. The application of linoleic acid is applicable to any transdermal procedure that results in impairing the ability of the skin to act as a protective layer. Indeed, most transdermal treatments have this effect as their function is to allow the active component to pass through the epidermis to the dermis at least, and, if systemic administration is achieved, through the dermis itself.

Additional therapeutic agents can be included in the compositions. For example, hydrocortisone or hydrocortisone acetate may be included in an amount ranging from 0.25% w/w to about 0.5% w/w. Menthol, phenol, and terpenoids, e.g., camphor, can be incorporated for cooling pain relief. For example, menthol can be included in an amount ranging from about 0.1% w/w to about 1.0% w/w.

The compositions containing anesthetics are useful for temporary relief of pain and itching associated with minor burns, cuts, scrapes, skin irritations, inflammation and rashes due to soaps, detergents or cosmetics, or, as noted above, pain associated with removal of fat deposits.

In some particular embodiments it is desirable to adjust the pH of a transdermal delivery formulation and the pH is adjusted to a level of pH 9-11 or 10-11, which can be done by providing appropriate buffers or simply adjusting the pH with base. In other embodiments, it is desirable to adjust the pH of a transdermal delivery formulation to a level of pH 4-6, which can be done by providing appropriate buffers or simply adjusting the pH with an acid.

In some applications a formulation for transdermal delivery may, for example, comprise: Aveeno®, for example in an amount from about 10 to about 95% w/w; from about 20 to about 85% w/w, from about 20 to about −75% w/w, from about 20 to about 50% w/w.

In another aspect, certain embodiments are directed to a sustained release drug delivery platform releases a therapeutic compound or compounds disclosed and made as a formulation described herein over a period of, without limitation, about 3 days after administration, about 7 days after administration, about 10 days after administration, about 15 days after administration, about 20 days after administration, about 25 days after administration, about 30 days after administration, about 45 days after administration, about 60 days after administration, about 75 days after administration, or about 90 days after administration. In other aspects of this embodiment, a sustained release drug delivery platform releases a therapeutic compound or compounds disclosed herein with substantially first order release kinetics over a period of, without limitation, at least 3 days after administration, at least 7 days after administration, at least 10 days after administration, at least 15 days after administration, at least 20 days after administration, at least 25 days after administration, at least 30 days after administration, at least 45 days after administration, at least 60 days after administration, at least 75 days after administration, or at least 90 days after administration.

The formulation described in this specification may also comprise more than one therapeutic compound as desired for the particular indication being treated, preferably those with complementary activities that do not adversely affect the other proteins. A transdermal delivery formulation to be used for in vivo administration can be sterile. This can be accomplished, for instance, without limitation, by filtration through sterile filtration membranes, prior to, or following, preparation of a transdermal delivery formulation or other methods known in the art, including without limitation, pasteurization.

Packaging and instruments for administration may be determined by a variety of considerations, such as, without limitation, the volume of material to be administered, the conditions for storage, whether skilled healthcare practitioners will administer or patient self-compliance, the dosage regime, the geopolitical environment (e.g., exposure to extreme conditions of temperature for developing nations), and other practical considerations.

In certain embodiments, kits can comprise, without limitation, one or more cream or lotion comprising one or more formulations described herein. In various embodiments, the kit can comprise formulation components for transdermal, topical, or subcutaneous administration, formulated to be administered as an emulsion coated patch. In all of these embodiments and others, the kits can contain one or more lotion, cream, patch, or the like in accordance with any of the foregoing, wherein each patch contains a single unit dose for administration to a subject.

Imaging components can optionally be included, and the packaging also can include written or web-accessible instructions for using a transdermal delivery formulation. A container can include, for example, a vial, bottle, patch, syringe, pre-filled syringe, tube or any of a variety of formats well known in the art for multi-dispenser packaging.

In an aspect, the concentration of poloxamer 407 in a transdermal delivery formulation is at least 10%, at least 15%, at least 20%, at least 25%, at least 28.75%, at least 30%, at least 35%, at least 40% or more. In an aspect, the concentration of poloxamer 407 in a transdermal delivery formulation is not more than 10%, not more than 15%, not more than 20%, not more than 25%, not more than 28.75%, not more than 30%, not more than 35%, not more than 40% or more. In an aspect, the concentration of poloxamer 407 in a transdermal delivery formulation is about 10%, about 15%, about 20%, about 25%, at least 28.75%, about 30%, about 35%, about 40% or more. In an aspect, the concentration of poloxamer 407 in a transdermal delivery formulation is from 10% to 40%, is from 15% to 35%, is from 20% to 30%, is from 25% to 30%, is from 28% to 29%.

A transdermal delivery formulation can include mixtures wherein the components interact synergistically and induce skin permeation enhancements better than that induced by the individual components. Synergies between chemicals can be exploited to design potent permeation enhancers that overcome the efficacy limitations of single enhancers. Several embodiments disclosed herein utilize three to five distinct permeation enhancers.

The transdermal delivery formulation may comprise a plurality of the following components: phosphatidylcholine, benzyl alcohol, isopropyl palmitate, stearic acid, cetyl alcohol, ethanol, safflower oil, oleic acid, almond oil, propylene glycol, deionized water, dextrose anhydrous, poloxamer 407, polyglyceryl-4 laurate, and a buffering agent and/or another active agent. The transdermal delivery formulation may comprise each of the following components: phosphatidylcholine, benzyl alcohol, isopropyl palmitate, stearic acid, cetyl alcohol, ethanol, safflower oil, oleic acid, almond oil, propylene glycol, deionized water, dextrose anhydrous, poloxamer 407, polyglyceryl-4 laurate, and a buffering agent and/or another active agent. A transdermal delivery formulation may comprise all but one, all but two, all but three, all but four, all but five, all but six, all but seven, all but eight, all but nine, or all but ten of the following ingredients: phosphatidylcholine, benzyl alcohol, isopropyl palmitate, stearic acid, cetyl alcohol, ethanol, safflower oil, oleic acid, almond oil, propylene glycol, deionized water, dextrose anhydrous, poloxamer 407, polyglyceryl-4 laurate, and a buffering agent and/or another active agent.

Certain components or ingredients of a transdermal delivery formulation provided herein may be supplemented with components described in greater detail in the inventor's related applications mentioned above, including PCT/US2022/012330, filed Jan. 13, 2022; PCT/US2020/038558, filed Jun. 18, 2020; PCT/US18/51250 filed Sep. 14, 2018; and PCT/US18/28017 filed Apr. 17, 2018. The contents of each of which is incorporated by reference in its entirety.

### Administration and Dosing.

Methods for treating, preventing or ameliorating a disease, disorder, a condition, or a symptom thereof or a condition related thereto are provided herein using a transdermal delivery formulation for transdermal delivery described herein. The methods provided herein may comprise or consist of topically administering one or more of a transdermal delivery formulation described herein to skin of a subject in need thereof. Preferred, but non-limiting embodiments are directed to methods for treating, preventing, inhibiting or ameliorating a disease, disorder, a condition, or a symptom described herein.

A formulation for transdermal delivery may comprise one or more medicaments (e.g., of Table 1 or may comprise medicaments comprising one or more buffering agents as disclosed herein) and a penetrant portion.

A transdermal delivery formulation provided herein can be topically administered in any form. For administration for the treatment of skin conditions a sufficient amount of the topical composition can be applied onto a desired area and surrounding skin, for example, in an amount sufficient to cover a desired skin surface. A transdermal delivery formulation can be applied to any skin surface, including for example, facial skin, and the skin of the hands, neck, chest and/or scalp.

In applying a transdermal delivery formulation of the invention, a transdermal delivery formulation itself is simply placed on the skin and spread across the surface and/or massaged to aid in penetration. The amount of transdermal delivery formulation used is typically sufficient to cover a desired surface area. In some embodiments, a protective cover is placed over the formulation once it is applied and left in place for a suitable amount of time, i.e., 5 minutes, 10 minutes, 20 minutes or more; in some embodiments an hour or two. The protective cover can simply be a bandage including a bandage supplied with a cover that is impermeable to moisture. This essentially locks in the contact of a transdermal delivery formulation to the skin and prevents distortion of a transdermal delivery formulation by evaporation in some cases. The composition may be applied to the skin using standard procedures for application such as a brush, a syringe, a gauze pad, a dropper, or any convenient applicator. More complex application methods, including the use of delivery devices, may also be used, but are not required. In an alternative to administering topically to intact skin, the surface of the skin may also be disrupted mechanically by the use of spring systems, laser powered systems, systems propelled by Lorentz force or by gas or shock waves including ultrasound and may employ microdermabrasion such as by the use of sandpaper or its equivalent or using microneedles or electroporation devices. Simple solutions of the agent(s) as well as the above-listed formulations that penetrate intact skin may be applied using occlusive patches, such as those in the form micro-patches. External reservoirs of the formulations for extended administration may also be employed.

In an alternative to administering topically to intact skin, the surface of the skin may also be disrupted mechanically by the use of spring systems, laser powered systems, use of iontophoresis, systems propelled by Lorentz force or by gas or shock waves including ultrasound and may employ microdermabrasion such as by the use of sandpaper or its equivalent or using microneedles or electroporation devices. Simple solutions of the agent(s) as well as the above-listed transdermal delivery formulations that penetrate intact skin may be applied using occlusive patches, such as those in the form of micro-patches. External reservoirs of the formulations for extended administration may also be employed.

Accordingly, in certain embodiments alternative methods of administering one or more therapeutic compounds or agents (e.g., medicaments) through intact skin are provided. As nonlimiting examples, these alternative methods might be selected from the following lists: on basis of working mechanism, spring systems, laser powered, energy-propelled, Lorentz force, gas/air propelled, shock wave (including ultrasound), on basis of type of load, liquid, powder, projectile, on basis of drug delivery mechanism, nano-patches, sandpaper (microdermabrasion), iontophoresis enabled, microneedles, on basis of site of delivery, intradermal, intramuscular, and subcutaneous injection. Other suitable delivery mechanisms include, without limitation, microneedle drug delivery, such as 3M Systems, Glide SDI (pushes drug as opposed to “firing” drug), MIT low pressure injectors, micropatches (single use particle insertion device), microelectro mechanical systems (MEMS), dermoelectroporation devices (DEP), transderm ion to system (DEP), TTS transdermal therapeutic systems, membrane-moderated systems (drug reservoir totally encapsulated in a shallow compartment), adhesive diffusion-controlled system (drug reservoir in a compartment fabricated from drug-impermeable metallic plastic backing), matrix dispersion type system (drug reservoir formed by homogeneously dispersing drug solids in a hydrophilic or lipophilic polymer matrix molder into medicated disc), and microreservoir system (combination of reservoir and matrix dispersion-type drug delivery system).

The application method is determined by the nature of the treatment but may be less critical than the nature of a transdermal delivery formulation itself. If the application is to a skin area, it may be helpful in some instances to prepare the skin by cleansing or exfoliation. In some instances, it is helpful to adjust the pH of the skin area prior to application of the formulation itself. The application of a transdermal delivery formulation may be by simple massaging onto the skin or by use of devices such as syringes or pumps. Patches could also be used. In some cases, it is helpful to cover the area of application to prevent evaporation or loss of a transdermal delivery formulation.

Where the application area is essentially skin, it is helpful to seal-off the area of application subsequent to supplying a transdermal delivery formulation and allowing the penetration to occur so as to restore the skin barrier. A convenient way to do this is to apply a composition comprising linoleic acid which effectively closes the entrance pathways that were provided by the penetrants of the invention. This application, too, is done by straightforward smearing onto the skin area or can be applied more precisely in measured amounts.

A transdermal delivery formulation can be applied in a single, one-time application, once a week, once a bi-week, once a month, or from one to twelve times daily, for a period of time sufficient to alleviate a condition, disease, disorder, symptoms, for example, for a period of time of one week, from 1 to 12 weeks or more, from 1 to 6 weeks, from 2 to 12 weeks, from 2 to 8 weeks, from 2 to 6 weeks, from 2 to 4 weeks, from 4 to 12 weeks, from 4 to 8 weeks, or from 4 to 6 weeks. The present compositions can be administered, for example, at a frequency of once per day to hourly if needed. The presently described formulations can be topically administered once or more per day for a period of time from 1 week to 4 weeks, of from 1 week to 2 weeks, for 1 week, for 2 weeks, for 3 weeks, or for 4 weeks or more. In some instances, it may also be desirable to continue treatment indefinitely, for example, to inhibit recurring inflammation. A suitable administration for a transdermal delivery formulation comprising a skin cream, lotion or ointment, for example is once, twice, three, four times daily, or hourly if needed.

As described above, if desired, other therapeutic agents can be employed in conjunction with those provided in the above-described compositions. The amount of active ingredients that may be combined with the carrier materials to produce a single dosage form will vary depending upon the host treated, the nature of the disease, disorder, or condition, and the nature of the active ingredients.

It is understood that a specific dose level for any particular patient will vary depending upon a variety of factors, including the activity of the specific active agent; the age, body weight, general health, sex and diet of the patient; the time of administration; the rate of excretion; possible drug combinations; the severity of the particular condition being treated; the area to be treated and the form of administration. One of ordinary skill in the art would appreciate the variability of such factors and would be able to establish specific dose levels using no more than routine experimentation.

Pharmacokinetic parameters such as bioavailability, absorption rate constant, apparent volume of distribution, unbound fraction, total clearance, fraction excreted unchanged, first-pass metabolism, elimination rate constant, half-life, and mean residence time can be determined by methods well known in the art.

A transdermal delivery formulation in accordance with the subject matter described herein may be a topical dosage form packaged in, for example, a multi-use or single-use package, including for example, a tube, a bottle, a pump, a container or bottle, a vial, ajar, a packet, or a blister package.

Single dosage kits and packages containing a once per day amount of the transdermal delivery formulation may be prepared. Single dose, unit dose, and once-daily disposable containers of the transdermal delivery formulation are also provided.

The present transdermal delivery formulation remains stable in storage for periods including up to about 5 years, from about 3 months to about 5 years, from about 3 months to about 4 years, from about 3 months to about 3 years, and alternately any time period from about 6 months to about 3 years.

A transdermal delivery formulation described herein remains stable for up to at least 3 years at a temperature of less than or equal to 40° C. In an embodiment, the presently described transdermal delivery formulation remains stable for at least 2 years at a temperature of less than or equal to 40° C. In an embodiment, the presently described transdermal delivery formulation remains stable for at least 3 years at a temperature of less than or equal to 40° C. and at a humidity of up to 75% RH, for at least 2 years at a temperature of less than or equal to 40° C. and at a humidity of up to 75% RH, or for at least 3 years at a temperature of less than or equal to 30° C. and at a humidity of up to 75% RH. In a further embodiment, the presently described transdermal delivery formulation in accordance with the subject matter described herein remains stable for an extended period of time when packaged in a multi-use container such as a bottle dispenser or the like, and exhibits equal to or even greater stability when packaged in a single-use package.

In another aspect, the transdermal delivery formulation of certain embodiments comprises a daily dose of particular buffering agent (e.g., Sodium Hydroxide (Sodium oxidanide), Sodium Bicarbonate (baking soda or Sodium hydrogen carbonate), Potassium Bicarbonate (potassium hydrogen carbonate or potassium acid carbonate), Lysine, Tris (Tromethamine, trisaminomethane, 2-amino-2-hydroxymethyl-propane-1,3-diol, or tris(hydroxymethyl)aminomethane), Calcium Carbonate, Sodium Carbonate (Disodium carbonate), Potassium Carbonate, Dipotassium Phosphate (Potassium phosphate dibasic or Potassium hydrogen phosphate), Disodium Phosphate (Sodium phosphate dibasic or Disodium hydrogen phosphate), Trisodium Phosphate, Meglumine ((2R,3R,4R,5S)-6-(Methylamino)hexane-1,2,3,4,5-pentol or Methylglucamine), Arginine, Triethanolamine (TEA or 2,2′,2″-Nitrilotriethanol), Glycine, Monosodium Phosphate (Sodium dihydrogen phosphate), Monopotassium Phosphate (Potassium dihydrogen phosphate), Tripotassium Phosphate (potassium phosphate), Monoethanolamine, Diethanolamine (Diolamine or 2-(2-hydroxyethylamino)ethanol), Magnesium carbonate, and 2-imidazole-1-yl-3-ethoxycarbonylpropionic acid (IEPA)) . . . ). A daily dose for topical or transdermal administration of a transdermal delivery formulation depends on the compound and animal and may be easily determined by the skilled artisan, a suitable amount is about 1 mg/kg to about 5 g/kg, and more typically the daily dose is about 10 mg/kg to about 5 g/kg, about 25 mg/kg to about 2000 mg/kg, about 50 mg/kg to about 2000 mg/kg, about 25 mg/kg to about 1000 mg/kg, about 50 mg/kg to about 1000 mg/kg, about 100 mg/kg to about 700 mg/kg, about 100 mg/kg to about 500 mg/kg, about 150 mg/kg to about 500 mg/kg, about 150 mg/kg to about 400 mg/kg, about 200 mg/kg to about 500 mg/kg, about 200 mg/kg to about 450 mg/kg, about 200 mg/kg to about 400 mg/kg, about 250 mg/kg to about 450 mg/kg, about 250 mg/kg to about 400 mg/kg, about 250 mg/kg to about 350 mg/kg, and about 275 mg/kg to about 325 mg/kg.

In various embodiments, the amount of buffering agent in the formulation is at least about 5% (w/w) to about 40% (w/w). In some embodiments, the amount of buffering agent in the formulation is at least about 10% (w/w) to about 40% (w/w). In some embodiments, the amount of buffering agent in the formulation is at least about 15% (w/w) to about 40% (w/w). In some embodiments, the amount of buffering agent in the formulation is at least about 20% (w/w) to about 40% (w/w). In some embodiments, the amount of buffering agent in the formulation is at least about 25% (w/w) to about 40% (w/w). In various embodiments, the amount of buffering agent in the formulation is at least about 5% (w/w) to about 35% (w/w). In various embodiments, the amount of buffering agent in the formulation is at least about 10% (w/w) to about 35% (w/w). In various embodiments, the amount of buffering agent in the formulation is at least about 15% (w/w) to about 35% (w/w). In various embodiments, the amount of buffering agent in the formulation is at least about 20% (w/w) to about 35% (w/w). In various embodiments, the amount of buffering agent in the formulation is at least about 25% (w/w) to about 35% (w/w).

In various embodiments, the amount of buffering agent in the formulation is at least 1% (w/w). In some embodiments, the amount of buffering agent in the formulation is at least 5% (w/w). In some embodiments, the amount of buffering agent in the formulation is at least 10% (w/w). In some embodiments, the amount of buffering agent in the formulation is at least 15% (w/w). In some embodiments, the amount of buffering agent in the formulation is at least 20% (w/w).

In various embodiments, the amount of buffering agent in the formulation is less than 40% (w/w). In various embodiments, the amount of buffering agent in the formulation is less than 35% (w/w). In various embodiments, the amount of buffering agent in the formulation is less than 30% (w/w). In various embodiments, the amount of buffering agent in the formulation is less than 25% (w/w). In various embodiments, the amount of buffering agent in the formulation is less than 20% (w/w). In various embodiments, the amount of buffering agent in the formulation is less than 15% (w/w). In various embodiments, the amount of buffering agent in the formulation is less than 10% (w/w).

In some cases, a combination of buffering agents is used. In some embodiments, the formulation comprises two buffering agents in equal amount, e.g., 15% sodium bicarbonate and 15% calcium carbonate or 5% Lysine and 5% IEPA. In other cases, the formulation comprises two buffering agents in differing amounts, e.g., 10% Tris and 15% magnesium carbonate or 7% Arginine and 3% glycine. In further cases, the formulation comprises three buffering agents in equal amounts, e.g., 5% sodium bicarbonate, 5% calcium carbonate, and 5% magnesium carbonate. In various cases, the formulation comprises three buffering agents in different amounts e.g., 5% IEPA, 3% sodium carbonate, and 2% monosodium phosphate or 7% tripotassium phosphate, 8% arginine, and 9% calcium carbonate. In other cases, the formulation comprises two or more buffering agents in equal amount. In various cases, the formulation comprises two or more buffering agents in different amount.

In various embodiments, the amount of the combination of buffering agents in the formulation is at least about 5% (w/w) to about 40% (w/w). In some embodiments, the amount of the combination of buffering agents in the formulation is at least about 10% (w/w) to about 40% (w/w). In some embodiments, the amount of the combination of buffering agents in the formulation is at least about 15% (w/w) to about 40% (w/w). In some embodiments, the amount of the combination of buffering agents in the formulation is at least about 20% (w/w) to about 40% (w/w). In some embodiments, the amount of the combination of buffering agents in the formulation is at least about 25% (w/w) to about 40% (w/w). In various embodiments, the amount of the combination of buffering agents in the formulation is at least about 5% (w/w) to about 35% (w/w). In various embodiments, the amount of the combination of buffering agents in the formulation is at least about 10% (w/w) to about 35% (w/w). In various embodiments, the amount of the combination of buffering agents in the formulation is at least about 15% (w/w) to about 35% (w/w). In various embodiments, the amount of the combination of buffering agents in the formulation is at least about 20% (w/w) to about 35% (w/w). In various embodiments, the amount of the combination of buffering agents in the formulation is at least about 25% (w/w) to about 35% (w/w).

In various embodiments, the amount of the combination of buffering agents in the formulation is at least 1% (w/w). In some embodiments, the amount of the combination of buffering agents in the formulation is at least 5% (w/w). In some embodiments, the amount of the combination of buffering agents in the formulation is at least 10% (w/w). In some embodiments, the amount of the combination of buffering agents in the formulation is at least 15% (w/w). In some embodiments, the amount of the combination of buffering agents in the formulation is at least 20% (w/w).

In various embodiments, the amount of the combination of buffering agents in the formulation is less than 40% (w/w). In various embodiments, the amount of the combination of buffering agents in the formulation is less than 35% (w/w). In various embodiments, the amount of the combination of buffering agents in the formulation is less than 30% (w/w). In various embodiments, the amount of the combination of buffering agents in the formulation is less than 25% (w/w). In various embodiments, the amount of the combination of buffering agents in the formulation is less than 20% (w/w). In various embodiments, the amount of the combination of buffering agents in the formulation is less than 15% (w/w). In various embodiments, the amount of the combination of buffering agents in the formulation is less than 10% (w/w). In some cases, a combination of buffering agents is used.

Buffering agents are especially useful medicaments for treating diseases or disorders that are local and/or superficial (e.g., relative to the skin surface), local, non-superficial (e.g., relative to the skin surface), or systemic disease with an inflammatory aspect, as disclosed elsewhere herein.

Using non-sodium buffering agents may help avoid hypernatremia, which is a high concentration of sodium in the blood, in a subject which could result when a high dosage of sodium-based buffering agent is administered and/or for a long period of time. In transdermal delivery formulations of the present disclosure comprising buffering agents, a combination of non-sodium and sodium buffering agents can be used. In some embodiments, the amount of the sodium salt in the buffering agent is 1%, 2%, 3%, 4%, 5%, 6%, 7%, 8%, 9%, 10%, 20%, 30%, 40%, 50%, 60%, 70%, or so of the amount of the non-sodium salts in the buffering agent.

If desired, other therapeutic agents can be employed in conjunction with those provided in the above-described compositions. The amount of active ingredients that may be combined with the carrier materials to produce a single dosage form will vary depending upon the host treated, the nature of the disease, disorder, or condition, and the nature of the active ingredients.

It is understood that a specific dose level for any particular patient will vary depending upon a variety of factors, including the activity of the specific active agent; the age, body weight, general health, sex and diet of the patient; the time of administration; the rate of excretion; possible drug combinations; the severity of the particular condition being treated; the area to be treated and the form of administration. One of ordinary skill in the art would appreciate the variability of such factors and would be able to establish specific dose levels using no more than routine experimentation.

Pharmacokinetic parameters such as bioavailability, absorption rate constant, apparent volume of distribution, unbound fraction, total clearance, fraction excreted unchanged, first-pass metabolism, elimination rate constant, half-life, and mean residence time can be determined by methods well known in the art.

A transdermal delivery formulation in accordance with the subject matter described herein may be a topical dosage form packaged in, for example, a multi-use or single-use package, including for example, a tube, a bottle, a pump, a container or bottle, a vial, ajar, a packet, or a blister package.

Single dosage kits and packages containing a once per day amount of the transdermal delivery formulation may be prepared. Single dose, unit dose, and once-daily disposable containers of the transdermal delivery formulation are also provided.

Alternatively, a suitable dose for topical or transdermal administration of each of one or more particular buffering agent (e.g. Sodium Hydroxide (Sodium oxidanide), Sodium Bicarbonate (baking soda or Sodium hydrogen carbonate), Potassium Bicarbonate (potassium hydrogen carbonate or potassium acid carbonate), Lysine, Tris (Tromethamine, trisaminomethane, 2-amino-2-hydroxymethyl-propane-1,3-diol, or tris(hydroxymethyl)aminomethane), Calcium Carbonate, Sodium Carbonate (Disodium carbonate), Potassium Carbonate, Dipotassium Phosphate (Potassium phosphate dibasic or Potassium hydrogen phosphate), Disodium Phosphate (Sodium phosphate dibasic or Disodium hydrogen phosphate), Trisodium Phosphate, Meglumine ((2R,3R,4R,5S)-6-(Methylamino)hexane-1,2,3,4,5-pentol or Methylglucamine), Arginine, Triethanolamine (TEA or 2,2′,2″-Nitrilotriethanol), Glycine, Monosodium Phosphate (Sodium dihydrogen phosphate), Monopotassium Phosphate (Potassium dihydrogen phosphate), Tripotassium Phosphate (potassium phosphate), Monoethanolamine, Diethanolamine (Diolamine or 2-(2-hydroxyethylamino)ethanol), Magnesium carbonate, 2-imidazole-1-yl-3-ethoxycarbonylpropionic acid (IEPA), or combination thereof.) for subject is at least about 100 mg, at least about 500 mg, at least about 1 g, at least about 5 g, at least about 10 g, at least about 15 g, at least about 16 g, at least about 17 g, at least about 18 g, at least about 19 g, at least about 20 g, at least about 21 g, at least about 22 g, at least about 23 g, at least about 24 g, at least about 25 g, at least about 26 g, at least about 27 g, at least about 28 g, at least about 29 g, at least about 30 g, at least about 35 g, at least about 40 g, at least about 45 g, at least about 50 g, at least about 60 g, at least about 75 g, at least about 100 g, at least about 200 g, at least about 500 g, or at least about 1.0 kg. This dose may be administered daily, twice a day, three times a day, four times a day, five times a day, or more than five times a day.

Aspects of the present specification disclose that the symptoms associated with a disease or disorder described herein are reduced following application of a transdermal delivery formulation by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% and the severity associated with a disease or disorder described herein is reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. Aspects of the present specification disclose the symptoms associated with disease or disorder are reduced following application of a transdermal delivery formulation by about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

Aspects of the present specification disclose that the symptoms associated with a disease or disorder described herein are reduced following administration of a transdermal delivery formulation of the present invention by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% and the severity associated with a disease or disorder described herein is reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. Aspects of the present specification disclose the symptoms associated with disease or disorder are reduced by about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

A transdermal delivery formulation as described herein can be used in the manufacture of medicaments and for the treatment of humans and other animals by administration in accordance with conventional procedures.

Dosing can be single dosage or cumulative (serial dosing), and can be readily determined by one skilled in the art. A transdermal delivery formulation of the present invention may be administered once, twice, three, four, five, six, seven, eight, nine, ten, eleven, twelve, thirteen, fourteen, fifteen, sixteen, seventeen, eighteen, nineteen, twenty or more times to a subject. For instance, treatment of a disease may comprise a one-time administration of an effective dose of a transdermal delivery formulation as disclosed herein. Alternatively, treatment of a disease may comprise multiple administrations of an effective dose of a transdermal delivery formulation as carried out over a range of time periods, such as, e.g., once daily, twice daily, thrice daily, once every few days, or once weekly. The timing of administration can vary from individual to individual, depending upon such factors as the severity of an individual's symptoms. For example, an effective dose of a transdermal delivery formulation as disclosed herein can be administered to an individual once daily for an indefinite period of time, or until the individual no longer requires therapy. A person of ordinary skill in the art will recognize that the condition of the individual can be monitored throughout the course of treatment and that the effective amount of a transdermal delivery formulation disclosed herein that is administered can be adjusted accordingly. In one embodiment, a transdermal delivery formulation as disclosed herein is capable of decreasing the time to resolve the symptoms of a disease, including in an individual suffering from a disease by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment.

In a further embodiment, a transdermal delivery formulation and its derivatives have half-lives of 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 1 week, 2 weeks, 3 weeks, 4 weeks, one month, two months, three months, four months or more.

In an embodiment, the period of administration of a transdermal delivery formulation is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In aspects of this embodiment, a therapeutically effective amount of a transdermal delivery formulation disclosed herein reduces or alleviates symptoms (e.g. muscle pain or cramping) of an ailment such as muscle cramping or soreness in an individual by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a therapeutically effective amount of a transdermal delivery formulation disclosed herein reduces or alleviates symptoms of an ailment in an individual by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a therapeutically effective amount of a transdermal delivery formulation disclosed herein reduces or alleviates symptoms of an ailment in an individual by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

A transdermal delivery formulation disclosed herein may comprise a transdermal delivery formulation in a therapeutically effective amount. As used herein, the term “effective amount” is synonymous with “therapeutically effective amount”, “effective dose”, or “therapeutically effective dose” and when used in reference to reducing or alleviate symptoms of an ailment in an individual refers to the minimum dose of a therapeutic disclosed herein necessary to achieve the desired therapeutic effect and includes a dose sufficient to reduce or alleviates symptoms of an ailment in an individual. The effectiveness of a transdermal delivery formulation disclosed herein capable of reducing or alleviating symptoms of an ailment in an individual can be determined by observing an improvement in an individual based upon one or more clinical symptoms, and/or physiological indicators associated with improvements in muscle performance, reduced soreness and/or overall health. Maintenance or a reduction of symptoms of an ailment can also be subjective to a patient. The effectiveness of a transdermal delivery formulation disclosed herein in an individual can be determined by observing an improvement in an individual based upon one or more clinical symptoms, and/or physiological indicators associated with signs/symptoms, muscle performance and general health. The effectiveness of a transdermal delivery formulation disclosed herein is also capable of prolonging the life of an individual as compared to the same individual if the transdermal delivery formulation is not administered. The effectiveness of the transdermal delivery formulation disclosed herein is also capable of enhancing the quality of life of an individual as compared to the same individual if the transdermal delivery formulation is not administered.

The appropriate effective amount of a transdermal delivery formulation disclosed herein to be administered to an individual can be determined by a person of ordinary skill in the art by taking into account factors, including, without limitation, an improvement in the individual based upon one or more clinical symptoms, and/or physiological indicators associated with improvements in muscle performance, reduced soreness and/or overall health, the particular characteristics, history and risk factors of the patient, such as, e.g., age, weight, general health and the like, or any combination thereof. Additionally, where repeated administration of a transdermal delivery formulation is used, an effective amount of a transdermal delivery formulation will further depend upon factors, including, without limitation, the frequency of administration, the half-life of the transdermal delivery formulation, or any combination thereof. It is known by a person of ordinary skill in the art that an effective amount of a transdermal delivery formulation disclosed herein can be extrapolated from in vitro assays and in vivo administration studies using animal models prior to administration to humans or animals.

Wide variations in the necessary effective amount are to be expected in view of the differing efficiencies of the various routes of administration. For instance, oral administration of a transdermal delivery formulation disclosed herein generally would be expected to require higher dosage levels than administration by inhalation. Similarly, systemic administration of a transdermal delivery formulation disclosed herein would be expected to require higher dosage levels than a local administration. Variations in these dosage levels can be adjusted using standard empirical routines of optimization, which are well-known to a person of ordinary skill in the art. The precise therapeutically effective dosage levels and patterns are preferably determined by the attending physician in consideration of the above-identified factors. One skilled in the art will recognize that the condition of the individual can be monitored throughout the course of therapy and that the effective amount of a therapeutic disclosed herein that is administered can be adjusted accordingly.

Aspects of the present specification disclose, in part, a reduction or alleviation of symptoms of an ailment such as muscle cramping or soreness in an individual. As used herein, the term “treating,” refers to reduction or alleviation of muscle soreness or cramping as well as improved muscle performance and recovery. For example, the term “treating” can mean reduction or alleviation of symptoms in an individual by, e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 100%. The actual symptoms associated with an ailment such as muscle cramping and soreness are well known and can be determined by a person of ordinary skill in the art by using commonly known testing means. Those of skill in the art will know the appropriate symptoms or indicators associated with health and muscle performance and will know how to determine if an individual is a candidate for treatment as disclosed herein.

In an embodiment, a first transdermal delivery formulation is administered to an individual and at a later date, a second transdermal delivery formulation is administered to the same individual. In an embodiment, a first transdermal delivery formulation is administered to an individual at the same time as a second transdermal delivery formulation is administered to the individual.

A transdermal delivery formulation as disclosed herein is administered to an individual. An individual is typically a human being, but can be an animal, including, but not limited to, dogs, cats, birds, cattle, horses, sheep, goats, reptiles and other animals, whether domesticated or not.

In one aspect, disclosed herein is a formulation for transdermal delivery of an active agent through the skin, nail or hair follicle of a subject, wherein the formulation comprises a) a transdermal delivery formulation in an amount less than about 60% w/w, comprising i. one or more phosphatides, ii. glucose, and iii. one or more fatty acids; and b) water in an amount less than about 50% w/w.

In some embodiments, the formulation comprises a) a transdermal delivery formulation in an amount less than about 60% w/w, comprising i. one or more phosphatides, ii. glucose, and iii. one or more fatty acids; and b) water in an amount less than about 50% w/w, further comprises benzyl alcohol in an amount from about 0.5 to about 5% w/w.

In some embodiments, the transdermal delivery formulation comprises benzyl alcohol in an amount less than 5% w/w of the formulation.

In some embodiments, the formulation comprises a) a transdermal delivery formulation in an amount less than about 60% w/w, comprising i. one or more phosphatides, ii. glucose, and iii. one or more fatty acids; and b) water in an amount less than about 50% w/w, further comprises Isopropyl Palmitate in an amount from about 5 to about −20% w/w.

In some embodiments, the water is deionized water and/or purified water.

In some embodiments, the water is in an amount from about 15 to about 40% w/w of the formulation.

In some embodiments, the one or more phosphatides in an amount from about 0.5 to about 55% w/w of the transdermal delivery formulation.

In some embodiments, the transdermal delivery formulation comprises phosphatidylcholine, hydrogenated phosphatidylcholine, phosphatidylserine, phosphatidylethanolamine, phosphatidylinositol, or a combination thereof in amount less than 30% w/w of the formulation.

In some embodiments, the one or more phosphatides comprises phosphatidylcholine of the transdermal delivery formulation.

In some embodiments, the one or more fatty acids in an amount from about 1 to about 35% w/w of the transdermal delivery formulation.

In some embodiments, the one or more fatty acids in an amount from about 5 to about 35% w/w of the transdermal delivery formulation.

In some embodiments, the one or more fatty acids comprises Linoleic Acid, Oleic Acid, Stearic Acid, safflower oil, or a combination thereof.

In some embodiments, the one or more fatty acids comprises Linoleic Acid.

In some embodiments, the one or more fatty acids comprises Oleic Acid.

In some embodiments, the one or more fatty acids comprises Stearic Acid.

In some embodiments, the one or more phosphatides are derived from a seed oil in an amount from about 0.5 to about 55% w/w of the transdermal delivery formulation.

In some embodiments, the one or more phosphatides are derived from a seed oil in an amount from about 5 to about 35% w/w of the transdermal delivery formulation.

In some embodiments, the one or more phosphatides are derived from a safflower oil in an amount from about 0.5 to about 55% w/w of the transdermal delivery formulation.

In some embodiments, the one or more phosphatides are derived from a safflower oil in an amount from about 5 to about 35% w/w of the transdermal delivery formulation.

In some embodiments, the one or more phosphatides are derived from an almond oil in an amount from about 0.5 to about 55% w/w of the transdermal delivery formulation.

In some embodiments, the one or more phosphatides are derived from an almond oil in an amount from about 5 to about 35% w/w of the transdermal delivery formulation.

In some embodiments, the one or more phosphatides comprises one or more fatty acids derived from soy lecithin.

In some embodiments, the glucose in an amount from about 0.05 to about 10% w/w of the transdermal delivery formulation.

In some embodiments, the glucose is anhydrous dextrose in an amount from about 0.05 to about 10% w/w of the transdermal delivery formulation.

In some embodiments, the formulation comprises a) a transdermal delivery formulation in an amount less than about 60% w/w, comprising i. one or more phosphatides, ii. glucose, and iii. one or more fatty acids; and b) water in an amount less than about 50% w/w, further comprises a nonionic surfactant in an amount from about 2 to about 25% w/w of the transdermal delivery formulation.

In some embodiments, the formulation comprises a) a transdermal delivery formulation in an amount less than about 60% w/w, comprising i. one or more phosphatides, ii. glucose, and iii. one or more fatty acids; and b) water in an amount less than about 50% w/w, further comprises a polar solvent at least in an amount in molar excess of the nonionic surfactant.

In some embodiments, the nonionic surfactant is a poloxamer and the polar solvent is water.

In some embodiments, the formulation comprises a) a transdermal delivery formulation in an amount less than about 60% w/w, comprising i. one or more phosphatides, ii. glucose, and iii. one or more fatty acids; and b) water in an amount less than about 50% w/w, further comprises a polar solvent in an amount less than 5% w/w of the formulation.

In some embodiments, the transdermal delivery formulation further comprises a detergent portion in an amount from about 1 to about 30% w/w of the transdermal delivery formulation.

In some embodiments, the detergent portion comprises a nonionic surfactant in an amount from about 2 to about 25% w/w of the transdermal delivery formulation; and a polar solvent in an amount less than 5% w/w of the transdermal delivery formulation.

In some embodiments, the transdermal delivery formulation is in an amount from about 10 to about 60% w/w of the transdermal delivery formulation.

In some embodiments, the transdermal delivery formulation comprises an alcohol in an amount less than 10% w/w of the transdermal delivery formulation.

In some embodiments, the transdermal delivery formulation further comprises an alcohol, a surfactant, and a polar solvent.

In some embodiments, the transdermal delivery formulation comprises cetyl alcohol in amount less than 5% w/w of the formulation.

In some embodiments, the transdermal delivery formulation comprises ethanol in an amount less than 5% w/w of the formulation.

In some embodiments, the transdermal delivery formulation comprises glycerine in an amount less than 5% w/w of the formulation.

In some embodiments, the transdermal delivery formulation comprises propylene glycol in an amount less than 8% w/w of the formulation.

In some embodiments, the formulation comprises a gelling agent in an amount less than 20% w/w of the formulation.

In some embodiments, the formulation comprises menthol in an amount from about 0.05 to about 5% w/w of the formulation.

In some embodiments, the formulation comprises a) a transdermal delivery formulation in an amount less than about 60% w/w, comprising i. one or more phosphatides, ii. glucose, and iii. one or more fatty acids; and b) water in an amount less than about 50% w/w.

In some embodiments, the formulation comprises a) a transdermal delivery formulation in an amount less than about 60% w/w, comprising i. one or more phosphatides, ii. glucose, and iii. one or more fatty acids; and b) water in an amount less than about 50% w/w, further comprises a humectant, an emulsifier, an emollient, or a combination thereof.

In some embodiments, the formulation has a pH of 9-11.

In some embodiments, the formulation has a pH of 7-10.5.

In some embodiments, the formulation comprises a) a transdermal delivery formulation in an amount less than about 60% w/w, comprising i. one or more phosphatides, ii. glucose, and iii. one or more fatty acids; and b) water in an amount less than about 50% w/w, further comprises an active agent.

In some embodiments, the formulation comprises a) a transdermal delivery formulation in an amount less than about 60% w/w, comprising i. one or more phosphatides, ii. glucose, iii. one or more fatty acids; b) water in an amount less than about 50% w/w, and an active agent component in an amount less than about 60% w/w.

In some embodiments, the formulation comprises a) a transdermal delivery formulation in an amount less than about 60% w/w, comprising i. one or more phosphatides, ii. glucose, and iii. one or more fatty acids; b) water in an amount less than about 50% w/w, and an active agent component in an amount less than about 60% w/w, wherein the active agent is a medicament.

In another aspect disclosed herein is a method to effect transdermal delivery of an active ingredient comprising applying to the skin, nails or hair follicles of a subject an effective amount of the formulation comprising a) a transdermal delivery formulation in an amount less than about 60% w/w, comprising i. one or more phosphatides, ii. glucose, and iii. one or more fatty acids; and b) water in an amount less than about 50% w/w, further comprises an active agent.

The transdermal delivery formulation may comprise a plurality of the following components: phosphatidylcholine, benzyl alcohol, isopropyl palmitate, stearic acid, cetyl alcohol, ethanol, safflower oil, oleic acid, almond oil, propylene glycol, deionized water, dextrose anhydrous, poloxamer 407, polyglyceryl-4 laurate, and a buffering agent and/or another active agent. The transdermal delivery formulation may comprise each of the following components: phosphatidylcholine, benzyl alcohol, isopropyl palmitate, stearic acid, cetyl alcohol, ethanol, safflower oil, oleic acid, almond oil, propylene glycol, deionized water, dextrose anhydrous, poloxamer 407, polyglyceryl-4 laurate, and a buffering agent and/or another active agent. A transdermal delivery formulation may comprise all but one, all but two, all but three, all but four, all but five, all but six, all but seven, all but eight, all but nine, or all but ten of the following ingredients: phosphatidylcholine, benzyl alcohol, isopropyl palmitate, stearic acid, cetyl alcohol, ethanol, safflower oil, oleic acid, almond oil, propylene glycol, deionized water, dextrose anhydrous, poloxamer 407, polyglyceryl-4 laurate, and a buffering agent and/or another active agent.

Certain components or ingredients of a transdermal delivery formulation provided herein may be supplemented with components described in greater detail in the inventor's related applications mentioned above, including PCT/US2022/012330, filed Jan. 13, 2022; PCT/US2020/038558, filed Jun. 18, 2020; PCT/US18/51250 filed Sep. 14, 2018; and PCT/US18/28017 filed Apr. 17, 2018. The contents of each of which is incorporated by reference in its entirety.

### Transdermal Delivery of Aqueous Insoluble Molecules.

Various transdermal delivery formulations of the present disclosure are able to successfully deliver (at relatively high bioavailability) a range of actives that are either entirely insoluble, at least partially, or majority insoluble. This ability to deliver insoluble actives is contrary to the commonly-held belief that actives generally need to be solubilized for transdermal drug delivery.

There are a number of pharmacologically active medicaments which are poorly soluble in in aqueous media and/or acidic solutions which have proved challenging to deliver to a subject either transdermally or orally, due to the inability of the drug to be absorbed by the small intestine, or inability of the medicament to be solubilized in a transdermal delivery formulation and penetrate the skin. In general, it has previously been thought that pharmacologically active medicaments must be wholly or partially dissolved in an aqueous solution in order to be absorbed by a subject and achieve a reasonably high bioavailability in a subject. In the case of orally administered medicaments, it is thought a drug must be wholly or partially soluble in an aqueous medium in order dissolved in the stomach and absorbed by the small intestine. In the case of transdermally administered drugs, it is thought that a medicament must be wholly or mostly soluble in the formulation in order to penetrate the skin and achieve a high or relatively high bioavailability in a subject.

Such medicaments which are poorly soluble in aqueous medium are drugs which fall into Class 2 and Class 4 of the Biopharmaceutical Classification System (BCS). The BCS is a scientific framework which predicts in vivo drug performance via in vitro measurements of solubility and permeation. Solubility is the extent a drug can dissolve in gastrointestinal (GI) fluid and permeation is the extent solubilized drug can pass across membranes of cells lining the GI tract. Medicaments have a high solubility according to BCS if less than 250 mL of an aqueous media (pH between 1-7.5) dissolves the highest API prescription dose. Class 2 drugs and Class 4 drugs have low solubilities as they do not meet this solubility criteria. Class 2 drugs have high permeability whereas class 4 drugs have low permeability.

Without wishing to be bound by theory, the mechanism of action in which the insoluble class 2 or class 4 medicaments are delivered at a high or reasonably high bioavailability transdermally may include one or more ingredients in the transdermal delivery formulations of present embodiments partially breaking down one or more layers of dermal tissue (e.g., stratum coreum, stratum lucidum, stratum granulosum, stratum spinosum, stratum basale) permitting the medicament to penetrate the dermis, and enter the targeted tissue at a high local concentration, providing a high or reasonably high bioavailability in the targeted tissue. In other embodiments, the mechanism of action may include one or more ingredients in the transdermal delivery formulations of present embodiments partially breaking down one or more layers of dermal tissue (e.g., stratum coreum, stratum lucidum, stratum granulosum, stratum spinosum, stratum basale) in order to permitting the medicament to penetrate the dermis, and enter the blood stream as to delivery an insoluble class 2 or class 4 medicaments systemically while bypassing the GI track.

Any herein-disclosed transdermal delivery formulation can provide systemic administration of a medicament (e.g., a medicament disclosed in Table 1 or a medicament comprising one or more buffering agents as disclosed herein) via transdermal delivery of the medicament.

### Combination Therapies.

In some aspects, the present disclosure provides a method for treating a disease or disorder or reducing a symptom thereof, in which the method comprises steps of administering to a subject in need thereof a herein disclosed transdermal delivery formulation and administering to the subject in need thereof a composition comprising one or more medicaments selected from Table 1 or a medicament comprising one or more buffering agents as disclosed herein.

In embodiments, the transdermal delivery formulation is administered before, contemporary with, or after the composition is administered.

In some embodiments, the amount of the one or more medicaments is the effective dose of the medicament as described in Table 1 a medicament comprising one or more buffering agents as disclosed herein.

In various embodiments, the composition is administered by the standard route for the one or more medicament, e.g., the standard route is oral, topical, enteral, parenteral, by intravenous injection or infusion, by intraperitoneal injection, by intramuscular injection, or by subcutaneous injection.

In embodiments, the composition is a liquid, a suspension, a gel, a geltab, a semisolid, a tablet, a sachet, a lozenge, a pill, or a capsule.

In some embodiments, a subject has a cancer and a transdermal delivery formulation of the present disclosure is administered along with the standard of care. Table 6 lists several small molecule inhibitors for cancer therapy along with their specific target and type of cancer and that may be combined with a medicament of Table 1 or a medicament comprising one or more buffering agents as disclosed herein. According to some embodiments, the following agents are administered transdermally.

TABLE 6

| Small Molecule Drug | Specific Target | Cancer Targeted |
|---|---|---|
| Imatinib (Gleevec) | Bcr-Abl | Philadelphia chromosome-positive chronic |
| | | myelogenous leukemia |
| Gefitinib (Iressa) | EGFR | Non-small cell lung cancer |
| Erlotinib (Tarceva) | EGFR | Non-small cell lung cancer, pancreatic cancer |
| Sunitinib (sutent) | VEGFR2, RET PDGFR, FLT- | Renal cell carcinoma |
| | 1, KIT, CSF-1 | |
| Lapatinib (Tykerb) | EGFR, HER2/neu | Breast cancer |
| Nilotinib (Tasigna) | Bcr-Abl, KIT, LCK | Chronic myelogenous leukemia |
| Sorafenib (Nexavar) | B-Raf, VEGFR2, EGFR, | Hepatocellular carcinoma |
| | PDGFR | |
| Temsirolimus (CCl-779) | mTOR | Renal cell carcinoma |
| Everolimus (afinitor) | mTOR | Renal cell carcinoma |
| Pazopanib (Votrient) | c-KIT, FGFR, PDGFR and | Renal cell carcinoma, soft tissue sarcoma |
| | VEGFR | |
| Crizotinib (Xalkori) | HGFR | Non-small cell lung cancer |
| Ruxolitinib (jafaki) | Jak1, Jak2 | Primary Myelofibrosis |
| Vandetenib (Caprelsa) | VEGFR, EGFR, RET | Medullary thyroid cancer |
| Axitinib (Inlyta) | VEGFR1-3, cKIT, PDGFR | Renal cell carcinoma |
| Bosutinib (Bosulif) | Src, Bcr-Abl | Philadelphia chromosome-positive chronic |
| | | myelogenous leukemia |
| Cabozantinib | c.Met, VEGFR2 | Medullary thyroid cancer |
| (Cometriz) | | |
| Ponatinib (lclusig) | Bcr-Abl | Chronic myeloid leukemia, acute lymphoblastic |
| | | leukemia |
| Regorafenib (Stivagra) | VEGFR1-3, c-KIT, TIE-2, | Metastatic colorectal cancer |
| | PDGFR-β, FGFR-1, RET, | |
| | Raf-1, B-RAF | |
| Ibrutinib (Imbruvica) | Bruton's tyrosine | Mantle cell lymphoma |
| | kinase | |
| Trametinib (Mekinist) | MEK | B-RAF 600E/K-mutant metastatic melanoma |
| Perifosine | Akt | Colorectal cancer, Multiple myeloma, Colon cancer |
| Bortezomib (Velcade) | 26S proteosome | Multiple myeloma |
| Carfilzomib (Kyprolis) | 20S proteosome | Multiple myeloma |
| Marizomib (NPI-0052) | 20S proteosome | Multiple myeloma |
| Batimastat (BB-94) | Broad spectrum | Various tumors |
| | MMPs | |
| Neovastat (AE-941) | MMPs 1, 9, 12; VEGFR-2 | Renal cell carcinoma, non-small cell lymphoma |
| Prinomastat (AG-3340) | MMPs 2, 3, 9, 13 and 14 | Renal cell carcinoma |
| Rebimastat (BMS- | MMPs 1, 2, 8, 9 and 14 | Advanced non small cell lung cancer |
| 275291) | | |
| Ganetespib | HSP 90 | Multiple cancers |
| NVP-AUY922 | HSP 90 | Various tumors |
| Marimastat (BB-2516) | Broad spectrum | |
| | MMPs | |
| Obatoclax (GX15-070) | Bcl-2 family of | Leukemia, lymphoma, myelofibrosis |
| | proteins | |
| Navitoclax (ABT-263) | Bcl-sL, Bcl-2 and Bcl-w | Lymphoma and chronic lymphocytic leukemia |
| LUT014 + EGFR | B-Raf and EGFR | Colorectal, melanoma, skin, lung, head & neck, |
| Inhibitor | | urinary bladder, pancreatic and breast cancer |

### Manufacturing Formulations.

A transdermal delivery formulation of the disclosure may be prepared in a number of ways. Typically, the components of a transdermal delivery formulation are simply mixed together in the required amounts. However, it is also desirable in some instances to, for example, carry out dissolution of a buffering agent and then add a separate preparation containing the components aiding the delivery of the buffering agent in the form of a carrier. The concentrations of these components in the carrier, then, will be somewhat higher than the concentrations required in a final transdermal delivery formulation. Thus, the buffering agent may first be dissolved in water and then added to a carrier comprising an alcohol, a transdermal delivery formulation and optionally a combination of a nonionic surfactant and polar gelling agent, or of ionic detergent. Alternatively, some subset of these components can first be mixed and then “topped off” with the remaining components either simultaneously or sequentially. The precise manner of preparing a transdermal delivery formulation will depend on the choice of buffering agent and the percentages of the remaining components that are desirable with respect to that buffering agent. In some embodiments, the water is in an amount from about 10 to about 85% w/w, from about 15 to about 50% w/w, or from about 15 to about 45% w/w of the formulation.

The transdermal delivery formulation is a multi-component mixture, whereby the particular concentrations of the penetration enhancers are informed in part by the molecular mass of the buffering agent and the therapeutic agent to be transported. A transdermal delivery formulation enables the buffering agent to become bio-available to the target site within minutes of topical administration. A transdermal delivery formulation permits the use of minimal concentrations of therapeutic agents, as little as 1/1000th of concentrations required of alternative processes, while enabling bioactivity and positive clinical outcomes simultaneously. In some embodiments, the transdermal delivery formulation comprises an alcohol in an amount less than 5% w/w of the formulation.

In some aspects, the buffering agent is Sodium Hydroxide (Sodium oxidanide), Sodium Bicarbonate (baking soda or Sodium hydrogen carbonate), Potassium Bicarbonate (potassium hydrogen carbonate or potassium acid carbonate), Lysine, Tris (Tromethamine, trisaminomethane, 2-amino-2-hydroxymethyl-propane-1,3-diol, or tris(hydroxymethyl)aminomethane), Calcium Carbonate, Sodium Carbonate (Disodium carbonate), Potassium Carbonate, Dipotassium Phosphate (Potassium phosphate dibasic or Potassium hydrogen phosphate), Disodium Phosphate (Sodium phosphate dibasic or Disodium hydrogen phosphate), Trisodium Phosphate, Meglumine ((2R,3R,4R,5S)-6-(Methylamino)hexane-1,2,3,4,5-pentol or Methylglucamine), Arginine, Triethanolamine (TEA or 2,2′,2″-Nitrilotriethanol), Glycine, Monosodium Phosphate (Sodium dihydrogen phosphate), Monopotassium Phosphate (Potassium dihydrogen phosphate), Tripotassium Phosphate (potassium phosphate), Monoethanolamine, Diethanolamine (Diolamine or 2-(2-hydroxyethylamino)ethanol), Magnesium carbonate, 2-imidazole-1-yl-3-ethoxycarbonylpropionic acid (IEPA), or combination thereof.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Reference in this specification to “one embodiment/aspect” or “an embodiment/aspect” means that a particular feature, structure, or characteristic described in connection with the embodiment/aspect is included in at least one embodiment/aspect of the disclosure. The use of the phrase “in one embodiment/aspect” or “in another embodiment/aspect” in various places in the specification are not necessarily all referring to the same embodiment/aspect, nor are separate or alternative embodiments/aspects mutually exclusive of other embodiments/aspects. Moreover, various features are described which may be exhibited by some embodiments/aspects and not by others. Similarly, various requirements are described which may be requirements for some embodiments/aspects but not other embodiments/aspects. Embodiment and aspect can in certain instances be used interchangeably.

As used in the specification and claims, the singular forms “a”, “an” and “the” include plural references unless the context clearly dictates otherwise.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” mean A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

As used herein, the term “about” a number refers to that number plus or minus 10% of that number and/or within one standard deviation (plus or minus) from that number. The term “about” a range refers to that range minus 10% of its lowest value and plus 10% of its greatest value and that range minus one standard deviation its lowest value and plus one standard deviation of its greatest value.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The terms “increased”, “increasing”, or “increase” are used herein to generally mean an increase by a statically significant amount relative to a reference level. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease in a value relative to a reference level. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level.

The term “subject” or “patient” refers to any single animal, more preferably a mammal (including such non-human animals as, for example, dogs, cats, horses, rabbits, zoo animals, cows, pigs, sheep, and non-human primates) for which treatment is desired. Most preferably, the patient herein is a human.

The term “medicament,” “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An active agent can be a primary active agent, or in other words, the component(s) of a composition to which the whole or part of the effect of the composition is attributed. An active agent can be a secondary agent, or in other words, the component(s) of a composition to which an additional part and/or other effect of the composition is attributed.

In an embodiment, a “pharmaceutical composition” is intended to include the combination of an active agent with a carrier, inert or active, in a sterile composition suitable for diagnostic or therapeutic use in vitro, in vivo or ex vivo. In one aspect, the pharmaceutical composition is substantially free of endotoxins or is non-toxic to recipients at the dosage or concentration employed.

In an embodiment, “an effective amount” refers to the amount of the defined component sufficient to achieve the desired chemical composition or the desired biological and/or therapeutic result. In an embodiment, that result can be the desired pH or chemical or biological characteristic, e.g., stability of the formulation. In other embodiments, the desired result is the alleviation or amelioration of the signs, symptoms, or causes of a disease, or any other desired alteration of a biological system. When the desired result is a therapeutic response, the effective amount will vary depending upon the specific disease or symptom to be treated or alleviated, the age, gender and weight of the subject to be treated, the dosing regimen of the formulation, the severity of the disease condition, the manner of administration and the like, all of which can be determined readily by one of skill in the art. A desired effect may, without necessarily being therapeutic, also be a cosmetic effect, in particular for treatment for disorders of the skin or muscles.

In an embodiment, as used herein, the terms “treating,” “treatment” and the like are used herein to mean obtaining a desired pharmacologic and/or physiologic effect. The effect may be prophylactic in terms of completely or partially preventing a disorder or sign or symptom thereof, and/or may be therapeutic in terms of amelioration of the symptoms of the disease or infection, or a partial or complete cure for a disorder and/or adverse effect attributable to the disorder.

The term “bioavailability” refers to the fraction of an administered dose of unchanged drug that reaches the systemic circulation. For example, when a medication is administered intravenously, its bioavailability is 100%. However, when a medication is administered via other routes (such as orally), its bioavailability generally decreases due to incomplete absorption and first-pass metabolism. Bioavailability is one of the essential tools in pharmacokinetics, as bioavailability must be considered when calculating dosages for non-intravenous routes of administration.

Additionally, the use of particular formulations can disrupt the balance of electrolytes and cations, including those such as the Na/K ratio. For example, the administration of formulations containing calcium carbonate can reduce the amount of sodium or other ions which can decrease the potential for reaching a hyponatremic state. Also, the use of calcium carbonate can also increase the serum levels of calcium which can reduce the amount of calcium leeched from the body by high sodium concentrations.

The formulations and methods of use provided herein take these complexities of electrolyte balance into account. One approach utilized herein in making formulations that avoid electrolyte imbalance and cation overload is to use non-metal buffers or buffers without counterions. Suitable buffering agents for these embodiments include Lysine (free base), TRIS, and IEPA.

For transdermal topical administration in particular for agents other than buffer, a suitable formulation typically involves a penetrant that enhances penetration of the skin and is, in some embodiments, composed of chemical permeation enhancers (CPEs). In some cases, it can also include peptides designed to penetrate cells i.e., cell penetrating peptides (CPPs) also known as skin penetrating peptides (SPPs). The formulation can be applied for example in the form of topical lotions, creams, and the like, as described herein. Many known and useful compounds and the like can be found in Remington's Pharmaceutical Sciences (13^{th} Ed), Mack Publishing Company, Easton, PA—a standard reference for various types of administration. As used herein, the term “formulation(s)” means a combination of at least one active ingredient with one or more other ingredient, also commonly referred to as excipients, which may be independently active or inactive. The term “formulation” may or may not refer to a pharmaceutically acceptable composition for administration to humans or animals and may include compositions that are useful intermediates for storage or research purposes.

For purposes herein, a formulation, a formulation for transdermal delivery and a transdermal delivery formulation are each a formulation for transdermal delivery, including, the transdermal delivery of an active ingredient for the treatment of a syndrome and or a disease in an individual.

The section headings used herein are for organizational purposes only and are not to be construed as limiting the subject matter described.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### EXAMPLES

The following non-limiting examples are provided for illustrative purposes only in order to facilitate a more complete understanding of representative embodiments now contemplated. These examples are intended to be a mere subset of all possible contexts in which the components of the formulation may be combined. Thus, these examples should not be construed to limit any of the embodiments described in the present specification, including those pertaining to the type and amounts of components of the formulation and/or methods and uses thereof.

In one embodiment, a transdermal delivery formulation disclosed herein is capable of reducing the signs/symptoms associated with an ailment in an individual suffering from the ailment by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment. In other aspects of this embodiment, an anti-cancer transdermal delivery formulation is capable of reducing the number of cancer cells or tumor size in an individual suffering from a cancer by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to a patient not receiving the same treatment.

In a further embodiment, a transdermal delivery formulation and its derivatives have half-lives of 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 1 week, 2 weeks, 3 weeks, 4 weeks, one month, two months, three months, four months or more.

In an embodiment, the period of administration of a transdermal delivery formulation is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In aspects of this embodiment, a therapeutically effective amount of a transdermal delivery formulation disclosed herein reduces or maintains signs/symptoms associated with an ailment in an individual by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a therapeutically effective amount of a transdermal delivery formulation disclosed herein reduces or maintains signs/symptoms associated with an ailment by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a therapeutically effective amount of a transdermal delivery formulation disclosed herein reduces or maintains signs/symptoms associated with an ailment by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

### Example 1: Transdermal Administration of Polidocanol

Polidocanol (CAS No. 9002-92-0) is small molecule that relieves itching caused by eczema and dry skin and to treat varicose veins, hemangiomas, and vascular malformations. In this example, a patient seeks treatment for a disease or disorder. A health care professional suggests that the patient try transdermal Polidocanol. Conventionally, it is administered at a dose of 2.5 mg-15 mg.

Polidocanol can be provided in a transdermal lotion or cream. The formulations described herein allow for effective transdermal administration of the compound. In this example, Polidocanol is included in the transdermal delivery formulation detailed herein.

Polidocanol Formulation

| Ingredient | Weight (%) |
|---|---|
| Phosphatidylcholine | 7.64% |
| Isopropyl Palmitate | 13.30% |
| Stearic Acid | 0.62% |
| Benzyl Alcohol | 1.39% |
| Carthamus Tinctorius | 2.93% |
| (safflower) oil | |
| Oleic Acid | 0.97% |
| Polyglyceryl-4 | 1.06% |
| Laurate | |
| Deionized Water | ~56% |
| Polidocanol | 0.25%-1.5% |
| Poloxamer 407 | 9.25% |
| Total | 100.00% |

Transdermal administration allows direct absorption into a specific area. For example, a lotion can be applied to an area close to where itching caused by eczema and dry skin and to treat varicose veins, hemangiomas, and vascular malformations is present.

The lotion can include a transdermal delivery formulation and the active agent (collectively referred to as the formulation). In this example, the dose of the active agent (i.e., Polidocanol) is 0.25% to 1.5% w/w or w/v of the solution. A transdermal medicament presents several benefits. The lack of interference with food and alcohol is one advantage. Topical delivery avoids the GI tract and can increase bioavailability. Increased bioavailability permits lower doses which reduce the risk of side effects. Polidocanol topical cream obviates the need for the invasive nature and side effects of the injection of drugs. Topical administration also allows for the patient to increase the volume and incidence of application based on need/symptoms.

A transdermal medicament is the ideal for several reasons. Polidocanol topical cream obviates the need for the invasive nature and side effects of the injection of drugs. Rather than return to a healthcare provider's office for injections, the patient can apply the agent directly through the skin. Topical administration also allows for the patient to apply the drug daily (or as prescribed) without the side-effects or oral or injection administration.

### Example 2: Transdermal Administration of MRTX1133

MRTX1133 (CAS No. 2621928-55-8) is a chemotherapeutic compound. MRTX1133 is a highly selective inhibitor of mutant KRAS G12D and can reversibly binds to the activated and inactivated KRAS G12D mutants and inhibit their activity. The specificity of MRTX1133 to KRAS G12D is more than 1000 times that of wild-type KRAS. MRTX1133 is a noncovalent, potent, and selective KRAS G12D inhibitor. MRTX1133 optimally fills the switch II pocket and extends three substituents to favorably interact with the protein, resulting in an estimated KD against KRAS G12D of 0.2 pM. MRTX1133 prevents SOS1-catalyzed nucleotide exchange and/or formation of the KRAS G12D/GTP/RAF1 complex, thereby inhibiting mutant KRAS-dependent signal transduction. MRTX1133 selectively inhibits KRAS G12D mutant, but not KRAS wild-type, tumor cells. MRTX1133 has single digit nanomolar activity in cellular assays and marked in vivo efficacy in tumor models harboring KRAS G12D mutations. In this example, a patient seeks treatment for a disease or disorder. A health care professional suggests that the patient try transdermal MRTX1133. Conventionally, it is administered at a dose of 60 mg-900 mg or 120 mg and 600 mg.

MRTX1133 can be provided in a transdermal lotion or cream. The formulations described herein allow for effective transdermal administration of the compound. In this example, MRTX1133 is included in the transdermal delivery formulation detailed herein.

MRTX1133 Formulation

| Ingredient | Weight (%) |
|---|---|
| Phosphatidylcholine | 7.64% |
| Isopropyl Palmitate | 13.30% |
| Stearic Acid | 0.62% |
| Benzyl Alcohol | 1.39% |
| Carthamus Tinctorius | 2.93% |
| (safflower) oil | |
| Oleic Acid | 0.97% |
| Polyglyceryl-4 | 1.06% |
| Laurate | |
| Deionized Water | ~27 to 51% |
| MRTX1133 | 6%-30% |
| Poloxamer 407 | 9.25% |
| Total | 100.00% |

Transdermal administration allows direct absorption into a specific area. For example, a lotion can be applied to an area close to where a cancer is present or may be administered anywhere on the body for systemic administration.

The lotion can include a transdermal delivery formulation and the active agent (collectively referred to as the formulation). In this example, the dose of the active agent (i.e., MRTX1133) is 6% to 30% w/w or w/v of the solution. A transdermal medicament presents several benefits. The lack of interference with food and alcohol is one advantage. Topical delivery avoids the GI tract and can increase bioavailability. Increased bioavailability permits lower doses which reduce the risk of side effects. MRTX1133 topical cream obviates the need for the invasive nature and side effects of the injection of drugs. Topical administration also allows for the patient to increase the volume and incidence of application based on need/symptoms.

A transdermal medicament is the ideal for several reasons. MRTX1133 topical cream obviates the need for the invasive nature and side effects of the injection of drugs. Rather than return to a healthcare provider's office for injections, the patient can apply the agent directly through the skin. Topical administration also allows for the patient to apply the drug daily (or as prescribed) without the side-effects or oral or injection administration.

### Example 3: Transdermal Administration of Incobotulinum Toxin A

Incobotulinum toxin A (XEOMIN®) is a neurotoxin. That is useful for treating upper limb spasticity, cervical dystonia, blepharospasm, chronic sialorrhea, upper limb spasticity in, pediatrics (excluding spasticity caused by cerebral palsy), chronic sialorrhea in pediatrics, and excessive saliva, or drooling in patients with Parkinson's Disease (PD)/parkinsonism. In this example, a patient seeks treatment for a disease or disorder. A health care professional suggests that the patient try transdermal MRTX1133. Conventionally, it is administered at a dose of 0.72 ng/mL-800 ng/mL.

Incobotulinum toxin A (XEOMIN®) can be provided in a transdermal lotion or cream. The formulations described herein allow for effective transdermal administration of the compound. In this example, MRTX1133 is included in the transdermal delivery formulation detailed herein.

Incobotulinum toxin A (XEOMIN ®)
Formulation

| Ingredient | Weight (%) |
|---|---|
| Phosphatidylcholine | 7.64% |
| Isopropyl Palmitate | 13.30% |
| Stearic Acid | 0.62% |
| Benzyl Alcohol | 1.39% |
| Carthamus Tinctorius | 2.93% |
| (safflower) oil | |
| Oleic Acid | 0.97% |
| Polyglyceryl-4 | 1.06% |
| Laurate | |
| Deionized Water | ~57% |
| Incobotulinum toxin A | 0.000072%- |
| (XEOMIN ®) | 0.08% |
| Poloxamer 407 | 9.25% |
| Total | 100.00% |

Transdermal administration allows direct absorption into a specific area. For example, a lotion can be applied to an area close to where a cancer is present or may be administered anywhere on the body for systemic administration.

The lotion can include a transdermal delivery formulation and the active agent (collectively referred to as the formulation). In this example, the dose of the active agent (i.e., Incobotulinum toxin A (XEOMIN®)) is 0.000072%-0.08% w/w or w/v of the solution. A transdermal medicament presents several benefits. The lack of interference with food and alcohol is one advantage. Topical delivery avoids the GI tract and can increase bioavailability. Increased bioavailability permits lower doses which reduce the risk of side effects. Incobotulinum toxin A (XEOMIN®) topical cream obviates the need for the invasive nature and side effects of the injection of drugs. Topical administration also allows for the patient to increase the volume and incidence of application based on need/symptoms.

A transdermal medicament is the ideal for several reasons. Incobotulinum toxin A (XEOMIN®) topical cream obviates the need for the invasive nature and side effects of the injection of drugs. Rather than return to a healthcare provider's office for injections, the patient can apply the agent directly through the skin. Topical administration also allows for the patient to apply the drug daily (or as prescribed) without the side-effects or oral or injection administration.

### Example 4 Transdermal Delivery Formulations of the Present Disclosure Provide Systemic Administration of Active Agents

In various embodiments, a transdermal delivery formulation comprises the components of

General Formulation

| Ingredient | Weight (%) |
|---|---|
| Phospholipid | 3-15% |
| Fatty acid ester | 5-20% |
| Long-chain fatty acids | 0.1-10% |
| Water | 30-90% |
| Medicament | 0.00005%- |
| | 30%. |
| Optionally: | |
| viscosity-improving | 5-20% |
| agent | |
| penetration | 0.5-5% |
| enhancer | |
| emulsifier | 0.5-10% |
| Total | 100.00% |

In the above table, where an ingredient has weight percent that ranges from 5 to 20% (e.g., for the Fatty acid ester and viscosity-improving agent) as an example, that ingredient may be present in the formulation at any percentage (w/w or w/v) from about 5% to about 20%. The weight percentage may be about 5% to about 20%. The weight percentage may be about 5% to about 6%, about 5% to about 7%, about 5% to about 8%, about 5% to about 9%, about 5% to about 10%, about 5% to about 11%, about 5% to about 12%, about 5% to about 13%, about 5% to about 14%, about 5% to about 15%, about 6% to about 7%, about 6% to about 8%, about 6% to about 9%, about 6% to about 10%, about 6% to about 11%, about 6% to about 12%, about 6% to about 13%, about 6% to about 14%, about 6% to about 15%, about 7% to about 8%, about 7% to about 9%, about 7% to about 10%, about 7% to about 11%, about 7% to about 12%, about 7% to about 13%, about 7% to about 14%, about 7% to about 15%, about 8% to about 9%, about 8% to about 10%, about 8% to about 11%, about 8% to about 12%, about 8% to about 13%, about 8% to about 14%, about 8% to about 15%, about 9% to about 10%, about 9% to about 11%, about 9% to about 12%, about 9% to about 13%, about 9% to about 14%, about 9% to about 15%, about 10% to about 11%, about 10% to about 12%, about 10% to about 13%, about 10% to about 14%, about 10% to about 15%, about 10% to about 16%, about 10% to about 17%, about 10% to about 18%, about 10% to about 19%, about 10% to about 20%, about 11% to about 12%, about 11% to about 13%, about 11% to about 14%, about 11% to about 15%, about 11% to about 16%, about 11% to about 17%, about 11% to about 18%, about 11% to about 19%, about 11% to about 20%, about 12% to about 13%, about 12% to about 14%, about 12% to about 15%, about 12% to about 16%, about 12% to about 17%, about 12% to about 18%, about 12% to about 19%, about 12% to about 20%, about 13% to about 14%, about 13% to about 15%, about 13% to about 16%, about 13% to about 17%, about 13% to about 18%, about 13% to about 19%, about 13% to about 20%, about 14% to about 15%, about 14% to about 16%, about 14% to about 17%, about 14% to about 18%, about 14% to about 19%, about 14% to about 20%, about 15% to about 16%, about 15% to about 17%, about 15% to about 18%, about 15% to about 19%, about 15% to about 20%, about 16% to about 17%, about 16% to about 18%, about 16% to about 19%, about 16% to about 20%, about 17% to about 18%, about 17% to about 19%, about 17% to about 20%, about 18% to about 19%, about 18% to about 20%, or about 19% to about 20%, and any range therebetween. The weight percentage may be about 5%, about 6%, about 7%, about 8%, about 9%, about 10%, about 11%, about 12%, about 13%, about 14%, about 15%, about 16%, about 17%, about 18%, about 19%, or about 20%. The weight percentage may be at least about 5%, about 6%, about 7%, about 8%, about 9%, about 10%, about 11%, about 12%, about 13%, or about 14%. The weight percentage may be at most about 6%, about 7%, about 8%, about 9%, about 10%, about 11%, about 12%, about 13%, about 14%, about 15%, about 16%, about 17%, about 18%, about 19%, or about 20%. Moreover, the weight percentage may be about 5% to about 6%. The weight percentage may be about 5% to about 5.1%, about 5% to about 5.2%, about 5% to about 5.3%, about 5% to about 5.4%, about 5% to about 5.5%, about 5% to about 5.6%, about 5% to about 5.7%, about 5% to about 5.8%, about 5% to about 5.9%, about 5% to about 6%, about 5.1% to about 5.2%, about 5.1% to about 5.3%, about 5.1% to about 5.4%, about 5.1% to about 5.5%, about 5.1% to about 5.6%, about 5.1% to about 5.7%, about 5.1% to about 5.8%, about 5.1% to about 5.9%, about 5.1% to about 6%, about 5.2% to about 5.3%, about 5.2% to about 5.4%, about 5.2% to about 5.5%, about 5.2% to about 5.6%, about 5.2% to about 5.7%, about 5.2% to about 5.8%, about 5.2% to about 5.9%, about 5.2% to about 6%, about 5.3% to about 5.4%, about 5.3% to about 5.5%, about 5.3% to about 5.6%, about 5.3% to about 5.7%, about 5.3% to about 5.8%, about 5.3% to about 5.9%, about 5.3% to about 6%, about 5.4% to about 5.5%, about 5.4% to about 5.6%, about 5.4% to about 5.7%, about 5.4% to about 5.8%, about 5.4% to about 5.9%, about 5.4% to about 6%, about 5.5% to about 5.6%, about 5.5% to about 5.7%, about 5.5% to about 5.8%, about 5.5% to about 5.9%, about 5.5% to about 6%, about 5.6% to about 5.7%, about 5.6% to about 5.8%, about 5.6% to about 5.9%, about 5.6% to about 6%, about 5.7% to about 5.8%, about 5.7% to about 5.9%, about 5.7% to about 6%, about 5.8% to about 5.9%, about 5.8% to about 6%, or about 5.9% to about 6%. The weight percentage may be about 5%, about 5.1%, about 5.2%, about 5.3%, about 5.4%, about 5.5%, about 5.6%, about 5.7%, about 5.8%, about 5.9%, or about 6%. The weight percentage may be at least about 5%, about 5.1%, about 5.2%, about 5.3%, about 5.4%, about 5.5%, about 5.6%, about 5.7%, about 5.8%, or about 5.9%. The weight percentage may be at most about 5.1%, about 5.2%, about 5.3%, about 5.4%, about 5.5%, about 5.6%, about 5.7%, about 5.8%, about 5.9%, or about 6%. Further, the weight percentage may be about 5% to about 5.1%. The weight percentage may be about 5% to about 5.01%, about 5% to about 5.02%, about 5% to about 5.03%, about 5% to about 5.04%, about 5% to about 5.05%, about 5% to about 5.06%, about 5% to about 5.07%, about 5% to about 5.08%, about 5% to about 5.09%, about 5% to about 5.1%, about 5.01% to about 5.02%, about 5.01% to about 5.03%, about 5.01% to about 5.04%, about 5.01% to about 5.05%, about 5.01% to about 5.06%, about 5.01% to about 5.07%, about 5.01% to about 5.08%, about 5.01% to about 5.09%, about 5.01% to about 5.1%, about 5.02% to about 5.03%, about 5.02% to about 5.04%, about 5.02% to about 5.05%, about 5.02% to about 5.06%, about 5.02% to about 5.07%, about 5.02% to about 5.08%, about 5.02% to about 5.09%, about 5.02% to about 5.1%, about 5.03% to about 5.04%, about 5.03% to about 5.05%, about 5.03% to about 5.06%, about 5.03% to about 5.07%, about 5.03% to about 5.08%, about 5.03% to about 5.09%, about 5.03% to about 5.1%, about 5.04% to about 5.05%, about 5.04% to about 5.06%, about 5.04% to about 5.07%, about 5.04% to about 5.08%, about 5.04% to about 5.09%, about 5.04% to about 5.1%, about 5.05% to about 5.06%, about 5.05% to about 5.07%, about 5.05% to about 5.08%, about 5.05% to about 5.09%, about 5.05% to about 5.1%, about 5.06% to about 5.07%, about 5.06% to about 5.08%, about 5.06% to about 5.09%, about 5.06% to about 5.1%, about 5.07% to about 5.08%, about 5.07% to about 5.09%, about 5.07% to about 5.1%, about 5.08% to about 5.09%, about 5.08% to about 5.1%, or about 5.09% to about 5.1%. The weight percentage may be about 5%, about 5.01%, about 5.02%, about 5.03%, about 5.04%, about 5.05%, about 5.06%, about 5.07%, about 5.08%, about 5.09%, or about 5.1%. The weight percentage may be at least about 5%, about 5.01%, about 5.02%, about 5.03%, about 5.04%, about 5.05%, about 5.06%, about 5.07%, about 5.08%, or about 5.09%. The weight percentage may be at most about 5.01%, about 5.02%, about 5.03%, about 5.04%, about 5.05%, about 5.06%, about 5.07%, about 5.08%, about 5.09%, or about 5.1%.

The other ranges (e.g., 3-15% for the phospholipids; 0.1-10% for the Long-chain fatty acids; 30-90% for the water; 0.05%-5% for the PDE5 inhibitor; 0.5-5% for the penetration enhancer; and 0.5-10% for the emulsifier) recited in the above table include similar ranges and subranges and values within ranges. The present disclosure contemplates all similar ranges and subranges and values within ranges for each ingredient included in a formulation.

In some cases, the medicament is in an amount from about 0.001% to about 0.01% w/w of the formulation, in an amount from about 0.011% to about 0.1% w/w of the formulation, in an amount from about 0.11% to about 1.0% w/w of the formulation, in an amount from about 1% to about 10% w/w of the formulation, in an amount from about 11% to about 20% w/w of the formulation, or in an amount from about 21% to about 30% w/w of the formulation. The present disclosure contemplates all similar ranges and subranges and values within ranges for the medicament or medicaments included in a formulation.

The medicament is selected from Table 1 or is a medicament comprising one or more buffering agents as disclosed herein. The amount of the medicament in is as recited in Table 1 or as described elsewhere herein for the buffering agents as disclosed herein.

In some cases, more than one medicament is included in a transdermal delivery formulation. In these cases, the first and the second medicament are as disclosed in Table 1 or the one or more buffering agents as disclosed herein and the amounts of the first and the second medicament is as recited in Table 1 or as described elsewhere herein for the buffering agents as disclosed herein.

The transdermal delivery formulation may comprise a plurality of the following components: phosphatidylcholine, benzyl alcohol, isopropyl palmitate, stearic acid, cetyl alcohol, ethanol, safflower oil, oleic acid, almond oil, propylene glycol, deionized water, dextrose anhydrous, poloxamer 407, polyglyceryl-4 laurate, and a buffering agent and/or another active agent. The transdermal delivery formulation may comprise each of the following components: phosphatidylcholine, benzyl alcohol, isopropyl palmitate, stearic acid, cetyl alcohol, ethanol, safflower oil, oleic acid, almond oil, propylene glycol, deionized water, dextrose anhydrous, poloxamer 407, polyglyceryl-4 laurate, and a buffering agent and/or another active agent. A transdermal delivery formulation may comprise all but one, all but two, all but three, all but four, all but five, all but six, all but seven, all but eight, all but nine, or all but ten of the following ingredients: phosphatidylcholine, benzyl alcohol, isopropyl palmitate, stearic acid, cetyl alcohol, ethanol, safflower oil, oleic acid, almond oil, propylene glycol, deionized water, dextrose anhydrous, poloxamer 407, polyglyceryl-4 laurate, and a buffering agent and/or another active agent.

### Example 5 Combination Therapies

Provided herein is a method for treating a disease or disorder or reducing a symptom thereof, in which the method comprises steps of administering to a subject in need thereof a herein disclosed transdermal delivery formulation (e.g., as disclosed in Example 4 and elsewhere herein) and administering to the subject in need thereof a composition comprising one or more medicaments selected from Table 1 or comprising buffering agents as disclosed herein.

The transdermal delivery formulation may be administered before, contemporary with, or after the composition is administered.

The amount of the one or more medicaments is the effective dose of the medicament as described in Table 1 or as described elsewhere herein for the buffering agents as disclosed herein.

The composition is administered by the standard route for the one or more medicament, e.g., the standard route is oral, topical, enteral, parenteral, by intravenous injection or infusion, by intraperitoneal injection, by intramuscular injection, or by subcutaneous injection.

The composition may be a liquid, a suspension, a gel, a geltab, a semisolid, a tablet, a sachet, a lozenge, a pill, or a capsule.

### Example 6: Transdermal Administration of Resveratrol

Resveratrol is a stilbenoid, a type of natural phenol, which may exhibit antioxidant properties, and may be produced by plants. Resveratrol exhibits antioxidant properties and be may be useful in reducing circulating free radicals, and treating or reducing inflammation or an inflammatory response, treating or preventing cardiovascular disease, treating or preventing high blood pressure, treating or preventing cancer, treating or preventing metabolic syndrome, treating or preventing cognitive related disorders, treating or preventing Alzheimer's or dementia, increasing insulin sensitivity, treating or preventing diabetes, treating or preventing an appearance of aging skin.

In this example, a patient seeks treatment for a disease or disorder. A health care professional suggests that the patient try transdermal resveratrol. Conventionally, it is administered at a dose of 60 mg-900 mg or 120 mg and 600 mg.

Resveratrol is be provided in a transdermal lotion or cream. The formulations described herein allow for effective transdermal administration of the compound. In this example, resveratrol is included in the transdermal delivery formulation detailed herein.

Resveratrol Formulation

| Ingredient | Weight (%) |
|---|---|
| Phosphatidylcholine | 7.64% |
| Isopropyl Palmitate | 13.30% |
| Stearic Acid | 0.62% |
| Benzyl Alcohol | 1.39% |
| Carthamus Tinctorius | 2.93% |
| (safflower) oil | |
| Oleic Acid | 0.97% |
| Polyglyceryl-4 | 1.06% |
| Laurate | |
| Deionized Water | ~27 to 51% |
| Resveratrol | 6%-30% |
| Poloxamer 407 | 9.25% |
| Total | 100.00% |

Transdermal administration allows direct absorption into a specific area. For example, a lotion can be applied to an area close to where a cancer is present or may be administered anywhere on the body for systemic administration.

The lotion can include a transdermal delivery formulation and the active agent (collectively referred to as the formulation). In this example, the dose of the active agent (i.e., resveratrol) is 6% to 30% w/w or w/v of the solution, for example, 20%. A transdermal medicament presents several benefits. The lack of interference with food and alcohol is one advantage. Topical delivery avoids the GI tract and can increase bioavailability. Increased bioavailability permits lower doses which reduce the risk of side effects. Resveratrol topical cream obviates the need for the invasive nature and side effects of the injection of drugs. Topical administration also allows for the patient to increase the volume and incidence of application based on need/symptoms.

A transdermal medicament is the ideal for several reasons. resveratrol topical cream obviates the need for the invasive nature and side effects of the injection of drugs. Rather than return to a healthcare provider's office for injections, the patient can apply the agent directly through the skin. Topical administration also allows for the patient to apply the drug daily (or as prescribed) without the side-effects or oral or injection administration.

### Example 7: Transdermal Administration of LUT014

LUT014 6-methyl-5-N-[3-(7H-purin-6-yl)pyridin-2-yl]-1-N-[3 (trifluoromethoxy) phenyl]isoquinoline-1,5-diamine) (CAS 2274819-46-2) is a B-Raf inhibitor with an IC50 of 11.7 nM. In this example, a subject is being treated with an EGFR inhibitor and is experiencing a side effect of the EGFR inhibitor, such as EGFR inhibitor induced acneiform lesions. The EGFR inhibitor induced acneiform lesions may be the result of the EGFR inhibiting the MAP Kinase pathway inhibition of wild type epithelial cells, inducing the lesions. Without being bound to a particular theory, LUT014 may counter the effect of EGFR Inhibitors on wild type epithelial cells by overriding MAP Kinase pathway inhibition of EGFRs. By reversing the inhibitory effect of EGFR inhibitors on downstream proteins in the skin cells, LUT014 may reduce dose-limiting acneiform lesions associated EGFR Inhibitors treatment.

In this example, a patient being treated with an EGFR seeks treatment for managing a side effect of the EGFR. A health care professional suggests that the patient try transdermal LUT014. Conventionally, it is administered at a dose of 10 mg-900 mg, or 120 mg and 600 mg.

LUT014 can be provided in a transdermal lotion or cream. The formulations described herein allow for effective transdermal administration of the compound. In this example, LUT014 is included in the transdermal delivery formulation detailed herein.

LUT014 Formulation

| Ingredient | Weight (%) |
|---|---|
| Phosphatidylcholine | 7.64% |
| Isopropyl Palmitate | 13.30% |
| Stearic Acid | 0.62% |
| Benzyl Alcohol | 1.39% |
| Carthamus Tinctorius | 2.93% |
| (safflower) oil | |
| Oleic Acid | 0.97% |
| Polyglyceryl-4 | 1.06% |
| Laurate | |
| Deionized Water | ~27 to 51% |
| LUT014 | 1%-30% |
| Poloxamer 407 | 9.25% |
| Total | 100.00% |

Transdermal administration allows direct absorption into a specific area. For example, a lotion can be applied to an area close to where a cancer is present or may be administered anywhere on the body for systemic administration.

The lotion can include a transdermal delivery formulation and the active agent (collectively referred to as the formulation). In this example, the dose of the active agent (i.e., LUT014) is 1% to 30% w/w or w/v of the solution, for example 2% w/w. A transdermal medicament presents several benefits. The lack of interference with food and alcohol is one advantage. Topical delivery avoids the GI tract and can increase bioavailability. Increased bioavailability permits lower doses which reduce the risk of side effects. LUT014 topical cream obviates the need for the invasive nature and side effects of the injection of drugs. Topical administration also allows for the patient to increase the volume and incidence of application based on need/symptoms.

A transdermal medicament is the ideal for several reasons. LUT014 topical cream obviates the need for the invasive nature and side effects of the injection of drugs. Rather than return to a healthcare provider's office for injections, the patient can apply the agent directly through the skin. Topical administration also allows for the patient to apply the drug daily (or as prescribed) without the side-effects or oral or injection administration.

Upon application of the transdermal LUT014 formulation, the subject experiences an improvement in the EGFR inhibitor induced acneiform lesions including one or more of a reduction in number of lesions, a reduction in lesion size, and reduction in lesion discharge, and healing of the lesions.

### Example 8: Transdermal Administration of LUT014

LUT014 6-methyl-5-N-[3-(7H-purin-6-yl)pyridin-2-yl]-1-N-[3 (trifluoromethoxy) phenyl]isoquinoline-1,5-diamine) (CAS 2274819-46-2) is a B-Raf inhibitor with an IC50 of 11.7 nM. In this example, a subject is being treated with radiation therapy and is experiencing radiation dermatitis. The radiation dermatitis may be the result of the radiation therapy producing an ionization event that lead to damage of cellular macromolecules, including double-stranded DNA breaks. For example, within the epidermis, this DNA damage may disrupt the normal proliferation and differentiation of basal keratinocytes, depleting the differentiated epidermal keratinocytes and ultimately resulting in the loss of the protective barrier provided by the skin. For instance, when combined with DNA damage disruption within the dermis, this may result in a complex set of effects including an immune response cascade, producing the radiation dermatitis. As the epidermis is comprised primarily of keratinocytes and undergoes a continual process of proliferative self-renewal, the proliferative self-renewal process may rely, in part, on continual signals for cell division and survival transmitted by the MAPK (ERK1/2) signaling pathway, and tight regulation of this pathway may a role in the control of normal skin development and homeostasis, the disruption of which may result in symptoms of radiation dermatitis. Without being bound to a particular theory, LUT014, as a B-Raf Inhibitor, may induce temporary hyperproliferation of cells to repopulate the epidermal keratinocytes and restore the skin barrier, for example, stimulating proliferation of the basal keratinocytes, driving the repopulation of the epidermal keratinocytes, and improving the time to dermatitis resolution.

In this example, a patient being treated with radiation therapy seeks treatment for managing radiation induced dermatitis. A health care professional suggests that the patient try transdermal LUT014. Conventionally, it is administered at a dose of 10 mg-900 mg, or 120 mg and 600 mg.

LUT014 can be provided in a transdermal lotion or cream. The formulations described herein allow for effective transdermal administration of the compound. In this example, LUT014 is included in the transdermal delivery formulation detailed herein.

LUT014 Formulation

| Ingredient | Weight (%) |
|---|---|
| Phosphatidylcholine | 7.64% |
| Isopropyl Palmitate | 13.30% |
| Stearic Acid | 0.62% |
| Benzyl Alcohol | 1.39% |
| Carthamus Tinctorius | 2.93% |
| (safflower) oil | |
| Oleic Acid | 0.97% |
| Polyglyceryl-4 | 1.06% |
| Laurate | |
| Deionized Water | ~27 to 51% |
| LUT014 | 1%-30% |
| Poloxamer 407 | 9.25% |
| Total | 100.00% |

Transdermal administration allows direct absorption into a specific area. For example, a lotion can be applied to an area close to where a cancer is present or may be administered anywhere on the body for systemic administration.

The lotion can include a transdermal delivery formulation and the active agent (collectively referred to as the formulation). In this example, the dose of the active agent (i.e., LUT014) is 1% to 30% w/w or w/v of the solution, for example, 2%. A transdermal medicament presents several benefits. The lack of interference with food and alcohol is one advantage. Topical delivery avoids the GI tract and can increase bioavailability. Increased bioavailability permits lower doses which reduce the risk of side effects. LUT014 topical cream obviates the need for the invasive nature and side effects of the injection of drugs. Topical administration also allows for the patient to increase the volume and incidence of application based on need/symptoms.

A transdermal medicament is the ideal for several reasons. LUT014 topical cream obviates the need for the invasive nature and side effects of the injection of drugs. Rather than return to a healthcare provider's office for injections, the patient can apply the agent directly through the skin. Topical administration also allows for the patient to apply the drug daily (or as prescribed) without the side-effects or oral or injection administration.

Upon application of the transdermal LUT014 formulation, the subject experiences an improvement in the radiation dermatitis, including, for example, a decrease in collagen production, improved skin elasticity, reduction of telangiectasia, reduction in redness, reduction in inflammation, a reduction in swelling, a reduction in blisters or skin ulcers, reduction in thinning or weakening of the skin, or combinations thereof.

### Example 9 Cancer Treatment Method

Provided herein is a method for treating a cancer, melanoma, or reducing a symptom thereof, in which the method comprises steps of administering to a subject in need thereof a herein disclosed transdermal delivery formulation (e.g., as disclosed in Example 4 and elsewhere herein) and administering to the subject in need thereof a composition comprising any one or more medicaments selected from Table 1, a medicament comprising one or more buffering agents as disclosed herein, or a medicament selected from Table 6; and, optionally, an immunotherapy, immunomodulatory agents or a chemotherapeutic agent.

Tumor cells create a microenvironment within and surrounding the tumor which improves the replication kinetics of cancer cells, which is detrimental to function and health of non-cancer cells, and which reduces the perfusion and efficacy of anti-cancer agents, including, for example, immunotherapies, immunomodulatory agents, and/or chemotherapies. Application of a herein disclosed transdermal delivery formulation induces a reduction in or neutralization of tumor acidity, and improves the perfusion, efficacy, and/or anti-tumor responses to immunotherapies, immunomodulatory agents, and/or chemotherapies.

### Mice

Female C57BL/6 mice (6-8 weeks old) are purchased from a commercial provider. Mice are humanely euthanized by CO2 inhalation according to the American Veterinary Medical Association Guidelines. Mice are observed daily and are humanely euthanized if a solitary subcutaneous tumor exceeded 200 cm2 in area or mice showed signs referable to metastatic cancer. All animal experiments are approved by the Institutional Animal Care and Use Committee and performed in accordance with the U.S. Public Health Service Policy and National Research Council Guidelines.

### Cell Lines and Cell Culture

B16 Melanoma, Yumm 1.1 Melanoma, cell lines were cultured and complete media RPMI media supplemented with 10% heat inactivated FBS, 0.1 millimole per liter non-essential amino acids, 1 millimole per liter sodium pyruvate, 2 millimoles per liter fresh L-glutamine, 100 milligram per milliliter streptomycin, 100 U/mL penicillin, 50 milligrams per milliliter gentamicin, 0.5 milligrams per milliliter fungizone, and 0.05 millimoles per liter 2-ME. The cell lines tested negative for mycoplasma contamination. All cell lines were passaged less than 10 times after initial revival from frozen stocks. All cell lines are validated in core facilities prior to use.

In vitro T-cell Culture

T cells are isolated from the spleens of pmel, OT-I, or OT-II mice using T-cell columns. Pmel T cells are cultured for 2 days in CM, containing 10 IU/mL IL2 and 5 mg/mL gp100₂₅₋₃₃peptide. T cells are collected and cultured in CM, pH 6.6 or 7.4, with gp100₂₅₋₃₃peptide (10 mcg/mL) for 24 hours. Freshly isolated OT-I T cells are cultured in CM, pH 6.6 or 7.4, with OVA_{SIINFEKL}peptide (10 mcg/mL) for 24 hours. Freshly isolated OT-II T cells are cultured in CM, pH 6.6 or 7.4, with OVA₃₂₃-339peptide (10 mcg/mL) for 24 hours. Freshly isolated T cell from TDAG8 KO and wild-type (WT) littermates are cultured with 5 mcg/mL plate-bound anti-CD3 antibodies for 48 hours. For restimulation experiments, pmel T cells are collected after 24 hours of culture in CM, pH 6.6 or pH 7.4, containing 10 mcg/mL gp100₂₅₋₃₃ peptide and recultured in CM, pH 7.4, with 10 mcg/mL gp100₂₅₋₃₃ peptide for 24 to 48 hours. Cell supernatants are collected and IFNg production was measured by ELISA. For measurement of IFNg production by flow cytometry, brefeldin A (1 mcg/mL) was added to the cell culture 4 hours prior to harvest. Cells are harvested and stained with live/dead Aqua (Invitrogen) to exclude dead cells. Surface staining was done with anti-mouse CD8 APC antibody, followed by intracellular staining with anti-mouse IFNg PE, and data are acquired on the LSR II and analyzed using FlowJo software.

### Treatment Model

CB57B1/6 will receive application of the transdermal delivery formulation 3 days prior to tumor injection, and will continue to receive application of the transdermal delivery formulation until the end of the trial. Control mice do not receive application of the transdermal delivery formulation.

Six to eight-week-old female C57BL/6 mice (3 mice/group) are inoculated subcutaneously on the left flank with 0.2 mL of a single suspension containing 1×10\hat{ }6 B16-F10 murine melanoma cells.

Change in tumor pH will be measured in small (200-300 mm3) and large (700-800 mm3) tumors. pH will be measured using chemical exchange saturation transfer (CEST) of iopamidol (isovue 370) at 0, 1, 2, and 4 hrs post formula's administration.

Mice receive intraperitoneal injections of 20 mg/kg of anti-PD1 or anti-CTLA4 antibodies on day 3 and continue to receive antibodies every 3 to 4 days until the end of the experiment. Mice were humanely euthanized when tumors exceeded 1.5 cm in diameter, appeared necrotic, or interfered with locomotion. Tumors are collected and weighed.

### Adoptive T-Cell Therapy

C57BL/6 mice application of the transdermal delivery formulation 3 days prior to tumor injection and continued to receive bicarbonate water until the end of the experiment. Control mice will not receive the transdermal delivery formulation. A total of 1×10\hat{ }6 B16 tumor cells are injected subcutaneously in the left flank of C57BL/6 mice. Three days later, mice receive a sublethal dose (600 cGy) of total body irradiation (TBI) administered by an X-ray irradiator. For adoptive transfer experiments, T cells are isolated from the spleens of pmel mice and cultured in media containing 10 IU/mL of IL2 and 5 mg/mL of gp100₂₅₋₃₃ in vitro for 5 days. On day 4 following tumor injection, 5×10\hat{ }6 T cells are intravenously injected. IL2 (2.5e5 IU) is given intraperitoneally following T-cell injection, continuing every 12 hours for 3 days, for a total of 6 injections. Following this treatment, tumor size is measured and recorded every 2 days.

### Flow Cytometry

Spleens and tumors are harvested under sterile conditions. Single-cell suspensions are prepared, and red blood cells are removed using ACK lysis buffer. Tumor cell suspensions are prepared from solid tumors by enzymatic digestion in Hank's Balanced Salt Solution (HBSS) containing 1 mg/mL collagenase, 0.1 mg/mL DNAse I, and 2.5 U/mL of hyaluronidase, with constant stirring for 2 hours at room temperature. The resulting suspension was passed through a 70-mm cell strainer and washed once with HBSS. Cells are resuspended in PBS [symbol] 3% BSA to a concentration of 0.5-1 106 cells/mL for flow cytometric analysis. After red blood cell lysis with ACK buffer, cells are stained in FACS buffer with the following antibodies for flow cytometric analysis: CD3, CD4, and CD8 for T-cell detection and CD11b, Ly6G, and Ly6C for MDSC detection. Live/Dead fixable near-IR or aqua fluorescent reactive dyes are used to exclude dead cells before analysis. Cells are acquired by LSR II equipped with four, and the data are analyzed with FlowJo.

### Intracellular pH Measurement

Pmel T cells are cultured at pH 7.4 in the presence of gp100₂₅₋₃₃ peptide for 24 hours. Cells are washed and resuspended in serum-free media and SNARF-1, acetoxymethyl ester, acetate was added to a final concentration of 10 mmol/L. After 30-minute incubation, cells are split into two aliquots that are washed two times with PBS at either pH 6.6 or 7.4. In the last wash, each of the two aliquots are again split into two aliquots at each pH and resuspended in either regular PBS at the respective pH or high-K PBS [symbol] 10 mmol/L nigericin at the respective pH. Samples are then analyzed by flow cytometry with laser excitation of approximately 543 nm and emissions at 540 and 680 nm. Ratios of fluorescence emission intensities of SNARF-1 at 580 and 640 nm, which are sensitive to pH, are then plotted.

qRT-PCT

NA is extracted using RNeasy Isolation Kit (Qiagen). qRT-PCR is then carried out using iTaq Universal SYBR Green One-Step Kit (Bio-Rad) using a primer sequence. Results are normalized using 36B4, then expressed as fold change: (FC)=2-ΔΔCt, where ΔΔCt=(CtTarget−Ct36B4)assay−(CtTarget−Ct36B4)control.

### Determination of PK and PD of Transdermal Delivery Formulations

PD is determined by
a. measuring the time- and dose-dependent changes in tumor pH following an up to 100 uL of the transdermal delivery formulation; and
b. measuring the time- and dose-dependent changes in tumor pH following up to 100 μL of transdermal delivery formulation t.i.d. for (three days and/or 10 days, or the entire course of therapy).

Measurement of the Efficacy of Improvement of Immunotherapy, Immunomodulatory agents or Chemotherapeutic agents.

Tumors will be induced s.c. by the injection of 1×10\hat{ }5 viable B16 cells in the flank of C57BL/6 mice. Three days after injection of tumor, mice will be prepared for ACT by treatment with 600 rad of total body irradiation (TBI) to induce lymphopenia/6. mice received up to 100 μL of transdermal delivery formulation t.i.d. for three days prior to tumor injection and continued to receive t.i.d. application of formulation until the end of the experiment. Control mice do not receive the transdermal delivery formulation. On day 4-10, mice will receive adoptive transfer of activated pmel T cells. Pmel T cells recognize the melanoma associated antigen gp100 and after five days of in vitro culture with gp100 peptide, 80% of pmel T cells express CTLA4 and PD1 (data not shown). We will inoculate 12 mice per group and will dose 8 of these that are closed to the mean tumor volume at the time of treatment. Mice will receive 1×10 \hat{ }6, 5×10\hat{ }6 or 1×10\hat{ }7 T cells. Experimental groups will also receive 300 μg of anti-CTLA4 or antiPD1 antibodies every 3-4 days for 3 weeks. Checkpoint inhibitors will be purchased from BioXcell. Control mice will receive NrIgG antibody. Subcutaneous tumors will be monitored every 2-3 days with Vernier calipers, and the largest perpendicular measurements of tumor area (in mm2) will be recorded. Comparisons of tumor size and survival will be made between groups. Other melanoma tumor lines established from patient's melanoma samples will be considered as a model. NOG-IL-2 mice will be implanted with human melanoma tumor cells. Once tumors reach 25-50 mm2, mice will receive HLA matched T cells alone or in combination with the transdermal delivery formulation. Control mice will receive no transdermal delivery formulation or the transdermal delivery formulation alone. Comparisons of tumor size and survival will be made between groups. This study will be performed with 2 different melanoma cell lines.

### Results

Mice show an increase in tumor pH from an acidic pH to a neutral or alkaline pH (e.g., 7-9). There is observed a decrease in tumor size, an increase in the translation of proinflammatory cytokines, a de-inhibition of glycolytic activation, an increase in T cell activation, a decrease in cancer cell proliferation, a decrease in the death of non-cancerous cells, and increased perfusion and activity of the immunotherapy, immunomodulatory agent, or chemotherapeutic agent within the tumor microenvironment. Control mice show a maintenance of an acidic tumor pH, maintenance of inhibition of translation of proinflammatory cytokines, maintenance of inhibition of glycolytic activation, maintenance of decrease in T cell activation, increase in tumor size, increase in cancer cell proliferation, increase in death of non-cancerous cells, and limited perfusion and activity of the immunotherapy, immunomodulatory agent, or chemotherapeutic agent within the tumor microenvironment.

Alternatively, other buffering agents, e.g., Sodium Hydroxide (Sodium oxidanide), Potassium Bicarbonate (potassium hydrogen carbonate or potassium acid carbonate), Lysine, Tris (Tromethamine, trisaminomethane, 2-amino-2-hydroxymethyl-propane-1,3-diol, or tris(hydroxymethyl)aminomethane), Calcium Carbonate, Sodium Carbonate (Disodium carbonate), Potassium Carbonate, Dipotassium Phosphate (Potassium phosphate dibasic or Potassium hydrogen phosphate), Disodium Phosphate (Sodium phosphate dibasic or Disodium hydrogen phosphate), Trisodium Phosphate, Meglumine ((2R,3R,4R,5S)-6-(Methylamino)hexane-1,2,3,4,5-pentol or Methylglucamine), Arginine, Triethanolamine (TEA or 2,2′,2″-Nitrilotriethanol), Glycine, Monosodium Phosphate (Sodium dihydrogen phosphate), Monopotassium Phosphate (Potassium dihydrogen phosphate), Tripotassium Phosphate (potassium phosphate), Monoethanolamine, Diethanolamine (Diolamine or 2-(2-hydroxyethylamino)ethanol), Magnesium carbonate, 2-imidazole-1-yl-3-ethoxycarbonylpropionic acid (IEPA), or combination thereof could have been used in lieu of sodium bicarbonate.

### Example 10 Local and/or Superficial Disease and Disorder Treatment Method

The transdermal formulations of the present disclosure can deliver an active agent into an animal's bloodstream and thereby provide treatment for local and/or superficial diseases or disorders. Transdermal formulations of the medicament described herein allows penetration of the active agent thru the skin and into the blood stream. The transdermal formulations of the present disclosure can result in systemic administration of an active agent, e.g., any medicament disclosed in Table 1 or one or more buffering agents as disclosed herein for treating local and/or superficial diseases and disorders effectively.

The transdermal formulations of the present disclosure can provide a higher concentration of a molecule (especially one that is insoluble) and can provide systemic administration for a molecule that would be poorly absorbed by the gut epithelium. Together, molecules that are not suitable for enteral delivery or are suitable but at a low dosage can be systemically administered via transdermal formulations of the present disclosure.

Additionally, the method may further include administering an additional anti-inflammatory agent described herein before administering the transdermal delivery formulation. The method may also include administering the anti-inflammatory agent contemporary with (e.g., at the same time) as the transdermal delivery formulation. The method may further include administering the anti-inflammatory agent after the transdermal delivery formulation.

The anti-inflammatory agent may be a steroidal anti-inflammatory agent such as hydrocortisone, hydrocortisone acetate, cortisone acetate, tixocortol pivalate, prednisolone, methylprednisolone, prednisone, amcinonide, budesonide, desonide, fluocinolone acetonide, fluocinonide, halcinonide, triamcinolone acetonide, beclometasone, betamethasone, dexamethasone, fluocortolone, halometasone, mometasone, ciclesonide, cortisone acetate, hydrocortisone aceponate, hydrocortisone acetate, hydrocortisone buteprate, hydrocortisone butyrate, hydrocortisone valerate, prednicarbate, or tixocortol pivalate.

The anti-inflammatory agent may be a non-steroidal anti-inflammatory agent such as acetylsalicylic acid, diflunisal, salicylic acid and its salts, salsalate, Ibuprofen, Dexibuprofen, Naproxen, Fenoprofen, Ketoprofen, Dexketoprofen, Flurbiprofen, Oxaprozin, Loxoprofen, Pelubiprofen, Zaltoprofen, Indomethacin, Tolmetin, Sulindac, Etodolac, Ketorolac, Diclofenac, Aceclofenac, Bromfenac, Nabumetone, Piroxicam, Meloxicam, Tenoxicam, Droxicam, Lornoxicam, Isoxicam, Phenylbutazone, Mefenamic acid, Meclofenamic acid, Flufenamic acid, Tolfenamic acid, Celecoxib, Refocoxib, Valdecoxib, Parecoxib, Lumiracoxic, Etoricoxib, Firocoxib, Nimesulide, Clonixin, or Licofelone.

Accordingly, any herein-disclosed transdermal formulation can provide systemic administration of a medicament via transdermal delivery of the medicament for treating a systemic disease with an inflammatory aspect.

### Example 11 Local, Non-Superficial Disease and Disorder Treatment Method

The transdermal formulations of the present disclosure can deliver an active agent into an animal's bloodstream and thereby provide treatment for local, non-superficial diseases or disorders. Transdermal formulations of the medicament described herein allows penetration of the active agent thru the skin and into the blood stream. The transdermal formulations of the present disclosure can result in systemic administration of an active agent, e.g., any medicament disclosed in Table 1 or one or more buffering agents as disclosed herein for treating local, non-superficial diseases and disorders effectively.

The transdermal formulations of the present disclosure can provide a higher concentration of a molecule (especially one that is insoluble) and can provide systemic administration for a molecule that would be poorly absorbed by the gut epithelium. Together, molecules that are not suitable for enteral delivery or are suitable but at a low dosage can be systemically administered via transdermal formulations of the present disclosure.

Additionally, the method may further include administering an additional anti-inflammatory agent described herein before administering the transdermal delivery formulation. The method may also include administering the anti-inflammatory agent contemporary with (e.g. at the same time) as the transdermal delivery formulation. The method may further include administering the anti-inflammatory agent after the transdermal delivery formulation.

The anti-inflammatory agent may be a steroidal anti-inflammatory agent such as hydrocortisone, hydrocortisone acetate, cortisone acetate, tixocortol pivalate, prednisolone, methylprednisolone, prednisone, amcinonide, budesonide, desonide, fluocinolone acetonide, fluocinonide, halcinonide, triamcinolone acetonide, beclometasone, betamethasone, dexamethasone, fluocortolone, halometasone, mometasone, ciclesonide, cortisone acetate, hydrocortisone aceponate, hydrocortisone acetate, hydrocortisone buteprate, hydrocortisone butyrate, hydrocortisone valerate, prednicarbate, or tixocortol pivalate.

The anti-inflammatory agent may be a non-steroidal anti-inflammatory agent such as acetylsalicylic acid, diflunisal, salicylic acid and its salts, salsalate, Ibuprofen, Dexibuprofen, Naproxen, Fenoprofen, Ketoprofen, Dexketoprofen, Flurbiprofen, Oxaprozin, Loxoprofen, Pelubiprofen, Zaltoprofen, Indomethacin, Tolmetin, Sulindac, Etodolac, Ketorolac, Diclofenac, Aceclofenac, Bromfenac, Nabumetone, Piroxicam, Meloxicam, Tenoxicam, Droxicam, Lornoxicam, Isoxicam, Phenylbutazone, Mefenamic acid, Meclofenamic acid, Flufenamic acid, Tolfenamic acid, Celecoxib, Refocoxib, Valdecoxib, Parecoxib, Lumiracoxic, Etoricoxib, Firocoxib, Nimesulide, Clonixin, or Licofelone.

Accordingly, any herein-disclosed transdermal formulation can provide systemic administration of a medicament via transdermal delivery of the medicament for treating a systemic disease with an inflammatory aspect.

### Example 12 Systemic Disease with an Inflammatory Aspect Treatment Method

The transdermal formulations of the present disclosure can deliver an active agent into an animal's bloodstream and thereby provide treatment for systemic administration of the agent for systemic diseases with an inflammatory aspect. Transdermal formulations of the medicament described herein allows penetration of the active agent thru the skin and into the blood stream. The transdermal formulations of the present disclosure can result in systemic administration of an active agent, e.g., any medicament disclosed in Table 1 or one or more buffering agents as disclosed herein.

The transdermal formulations of the present disclosure can provide a higher concentration of a molecule (especially one that is insoluble) and can provide systemic administration for a molecule that would be poorly absorbed by the gut epithelium. Together, molecules that are not suitable for enteral delivery or are suitable but at a low dosage can be systemically administered via transdermal formulations of the present disclosure.

Additionally, the method may further include administering an additional anti-inflammatory agent described herein before administering the transdermal delivery formulation. The method may also include administering the anti-inflammatory agent contemporary with (e.g. at the same time) as the transdermal delivery formulation. The method may further include administering the anti-inflammatory agent after the transdermal delivery formulation.

The anti-inflammatory agent may be a steroidal anti-inflammatory agent such as hydrocortisone, hydrocortisone acetate, cortisone acetate, tixocortol pivalate, prednisolone, methylprednisolone, prednisone, amcinonide, budesonide, desonide, fluocinolone acetonide, fluocinonide, halcinonide, triamcinolone acetonide, beclometasone, betamethasone, dexamethasone, fluocortolone, halometasone, mometasone, ciclesonide, cortisone acetate, hydrocortisone aceponate, hydrocortisone acetate, hydrocortisone buteprate, hydrocortisone butyrate, hydrocortisone valerate, prednicarbate, or tixocortol pivalate.

The anti-inflammatory agent may be a non-steroidal anti-inflammatory agent such as acetylsalicylic acid, diflunisal, salicylic acid and its salts, salsalate, Ibuprofen, Dexibuprofen, Naproxen, Fenoprofen, Ketoprofen, Dexketoprofen, Flurbiprofen, Oxaprozin, Loxoprofen, Pelubiprofen, Zaltoprofen, Indomethacin, Tolmetin, Sulindac, Etodolac, Ketorolac, Diclofenac, Aceclofenac, Bromfenac, Nabumetone, Piroxicam, Meloxicam, Tenoxicam, Droxicam, Lornoxicam, Isoxicam, Phenylbutazone, Mefenamic acid, Meclofenamic acid, Flufenamic acid, Tolfenamic acid, Celecoxib, Refocoxib, Valdecoxib, Parecoxib, Lumiracoxic, Etoricoxib, Firocoxib, Nimesulide, Clonixin, or Licofelone.

Accordingly, any herein-disclosed transdermal formulation can provide systemic administration of a medicament via transdermal delivery of the medicament for treating a systemic disease with an inflammatory aspect.

### Example 13 RAS/MAPK Driven Tumor Treatment Method

In this example, RAS/MAPK Driven tumors can be treated with a transdermal delivery formulation described herein. Transdermal formulations of the medicament described herein allows penetration of the active agent thru the skin and into the blood stream. The transdermal formulations of the present disclosure can result in systemic administration of an active agent, e.g., any medicament disclosed in Table 1 or one or more buffering agents as disclosed herein.

The transdermal formulations of the present disclosure can provide a higher concentration of a molecule (especially one that is insoluble) and can provide systemic administration for a molecule that would be poorly absorbed by the gut epithelium. Together, molecules that are not suitable for enteral delivery or are suitable but at a low dosage can be systemically administered via transdermal formulations of the present disclosure.

Additionally, the method may further include administering an additional anti-inflammatory agent described herein before administering the transdermal delivery formulation. The method may also include administering the anti-inflammatory agent contemporary with (e.g., at the same time) as the transdermal delivery formulation. The method may further include administering the anti-inflammatory agent after the transdermal delivery formulation.

Additionally, naporafenib can be administered in this method, either as one of the medicaments of the transdermal formulation, administered via its normal route through intravenous injection or oral dosage, or in combination with the transdermal formulation comprising a buffering agent.

Accordingly, any herein-disclosed transdermal formulation can provide treatment of RAS/MAPK driven tumors.

### Example 14 RAS/MAPK Driven Tumor Treatment Method with Chemotherapeutic Agents

In this example, RAS/MAPK Driven tumors can be treated with a transdermal delivery formulation described herein in combination with a chemotherapeutic agent. Transdermal formulations of the medicament described herein allows penetration of the active agent thru the skin and into the blood stream. The transdermal formulations of the present disclosure can result in systemic administration of an active agent, e.g., any medicament disclosed in Table 1 or one or more buffering agents as disclosed herein. The combination with chemotherapeutic agents may have beneficial effects.

The transdermal formulations of the present disclosure can provide a higher concentration of a molecule (especially one that is insoluble) and can provide systemic administration for a molecule that would be poorly absorbed by the gut epithelium. Together, molecules that are not suitable for enteral delivery or are suitable but at a low dosage can be systemically administered via transdermal formulations of the present disclosure.

The transdermal formulation in this example may comprise a buffering agent and may be administered with a chemotherapeutic agent. Chemotherapeutic agents in this example for treatment with a transdermal formulation comprising a buffering agent may include mechlorethamine, chlorambucil, ifosfamide, melphalan, busulfan, carmustine, lomustine, procarbazine, dacardazine, cisplatin, carboplatin, mitomycin C, cyclophosphamide, ifosfamide, thiotepa, and dacarbazine, and analogues thereof.

Additionally, naporafenib can be administered in this method, either as one of the medicaments of the transdermal formulation, administered via its normal route through intravenous injection or oral dosage, or in combination with the transdermal formulation comprising a buffering agent.

Accordingly, any herein-disclosed transdermal formulation in combination with chemotherapeutic agents can provide treatment of RAS/MAPK driven tumors.

What is claimed is:

1. A formulation for transdermal delivery of a medicament through the skin of a subject, the formulation comprising a therapeutically effective amount of a medicament and a penetrant portion,
wherein the penetrant portion comprises: a phospholipid, a fatty acid ester formed from a low molecular weight alcohol, and a long-chain fatty acids, and, optionally, one or more of a viscosity-improving agent, a penetration enhancer, and an emulsifier.

2. The formulation of claim 1, wherein the phospholipid is selected from phosphatidylcholine, hydrogenated phosphatidylcholine, phosphatidylserine, phosphatidylethanolamine, phosphatidylinositol, inositol phosphatide, and sphingomyelin.

3. The formulation of claim 2, wherein the phospholipid is phosphatidylcholine.

4. The formulation of any one of claims 1 to 3, wherein the penetrant portion comprises two or more phospholipids.

5. The formulation of any one of claims 1 to 4, wherein the phospholipid is in an amount from about 3% to about 15% w/w of the formulation.

6. The formulation of any one of claims 1 to 5, wherein the low molecular weight alcohol is selected from isopropanol, methanol, ethanol, butanol, glycerol, cetyl alcohol.

7. The formulation of any one of claims 1 to 6, wherein the low molecular weight alcohol is isopropanol.

8. The formulation of any one of claims 1 to 7, wherein the fatty acid ester is selected from isopropyl palmitate, isopropyl myristate, isopropyl linoleate, isopropyl oleate, ethyl laurate, and ethyl myristate.

9. The formulation of claim 8, wherein the fatty acid ester is isopropyl palmitate.

10. The formulation of any one of claims 1 to 9, wherein the penetrant portion comprises two or more fatty acid esters.

11. The formulation of any one of claims 1 to 10, wherein the fatty acid ester is in an amount from about 5% to about 20% w/w of the formulation.

12. The formulation of any one of claims 1 to 11, wherein the long-chain fatty acid is selected from a linoleic, oleic, stearic acid, linolenic, palmitic, arachidonic, palmitoleic, myristic, eicosenoic, benehic, euricic, and lignoceric acid.

13. The formulation of claim 12, wherein the long-chain fatty acid is linoleic acid.

14. The formulation of claim 12, wherein the long-chain fatty acid is oleic acid.

15. The formulation of claim 12, wherein the long-chain fatty acid is stearic acid.

16. The formulation of any one of claims 12 to 15, wherein the long-chain fatty acid is obtained from safflower oil or almond oil.

17. The formulation of any one of claims 1 to 16, wherein the long-chain fatty acid is in an amount from about 0.1% to about 10% w/w of the formulation.

18. The formulation of any one of claims 1 to 17, wherein the penetrant portion comprises two or more long-chain fatty acids.

19. The formulation of any one of claims 1 to 18, wherein the penetrant portion comprises a viscosity-improving agent.

20. The formulation of any one of claims 1 to 19, wherein the viscosity-improving agent is a poloxamer.

21. The formulation of claim 20, wherein the poloxamer is selected from poloxamer 407, poloxamer 188, poloxamer 184, and poloxamer 124.

22. The formulation of any one of claims 1 to 21, wherein the viscosity-improving agent is a surfactant.

23. The formulation of claim 22, wherein the surfactant is selected from sodium lauryl sulfate (sodium dodecyl sulfate); polyoxyethylated castor oil derivatives such as HCO-60 surfactant; nonoxynol; octoxynol; phenylsulfonate; poloxamers such as Pluronic® F68, Pluronic® F127, and Pluronic® L62; polyoleates; Rewopal® HVIO, sodium laurate, sodium oleate; sorbitan dilaurate; sorbitan dioleate; sorbitan monolaurate such as Span® 20; sorbitan monooleates; sorbitan trilaurate; sorbitan trioleate; sorbitan monopalmitate such as Span® 40; sorbitan stearate such as Span® 85; polyethylene glycol nonylphenyl ether such as Synperonic® NP; p-(1,1,3,3-tetramethylbutyl)-phenyl ether such as Triton™ X-100; and polysorbates such as polyoxyethylene (20) sorbitan monolaurate such as Tween® 20, polysorbate 40 (polyoxyethylene (20) sorbitan monopalmitate) such as Tween® 40, polysorbate 60 (polyoxyethylene (20) sorbitan monostearate) such as Tween® 60, polysorbate 80 (polyoxyethylene (20) sorbitan monooleate) such as Tween® 80, and polyoxyethylenesorbitan trioleate such as Tween® 85.

24. The formulation of claim 23, wherein the surfactant is sodium lauryl sulfate.

25. The formulation of any one of claims 1 to 24, wherein the penetrant portion comprises two or more viscosity-improving agents.

26. The formulation of any one of claims 1 to 25, wherein the viscosity-improving agent is in an amount from about 5% to about 20% w/w of the formulation.

27. The formulation of any one of claims 1 to 26, wherein the penetrant portion comprises a penetration enhancer.

28. The formulation of any one of claims 1 to 27, wherein the penetration enhancer is an alcohol or a terpene.

29. The formulation of claim 28, wherein the penetration enhancer as an alcohol is selected from benzyl alcohol, ethanol, propylene glycol, and polyethylene glycol.

30. The formulation of claim 29, wherein the penetration enhancer is benzyl alcohol.

31. The formulation of any one of claims 28 to 30, wherein the penetration enhancer as a terpene is selected from limonene, menthol, borneol, and camphor.

32. The formulation of any one of claims 27 to 31, wherein the penetration enhancer further acts as a preservative.

33. The formulation of any one of claims 1 to 32, wherein the penetrant portion comprises two or more penetration enhancers.

34. The formulation of any one of claims 1 to 33, wherein the penetration enhancer is in an amount from about 0.5% to about 5% w/w of the formulation.

35. The formulation of any one of claims 1 to 34, wherein the penetrant portion comprises at least one penetration enhancer and at least one viscosity-improving agent.

36. The formulation of any one of claims 1 to 35, wherein the penetrant portion comprises an emulsifier.

37. The formulation of any one of claims 1 to 36, wherein the emulsifier is selected from polyglyceryl-4-laurate, polyglyceryl-4-oleate, span 60, cetyl alcohol, and polyglyceryl-3-oleate.

38. The formulation of any one of claims 1 to 37, wherein the penetrant portion comprises two or more penetration enhancers.

39. The formulation of any one of claims 1 to 38, wherein the emulsifier is in an amount from about 0.5 to about 10% w/w of the formulation.

40. The formulation of any one of claims 1 to 39, wherein the penetrant portion comprises at least one emulsifier and at least one viscosity-improving agent.

41. The formulation of any one of claims 1 to 40, wherein the penetrant portion comprises at least one emulsifier and at least one penetration enhancer.

42. The formulation of any one of claims 1 to 41, wherein the penetrant portion comprises at least one emulsifier, at least one viscosity-improving agent, and at least one penetration enhancer.

43. A formulation for transdermal delivery of a medicament through the skin of a subject, the formulation comprising a therapeutically effective amount of a medicament and a penetrant portion,
wherein the penetrant portion comprises: a phospholipid, a fatty acid ester formed from a low molecular weight alcohol, and a long-chain fatty acid,
wherein phosphatidylcholine, hydrogenated phosphatidylcholine, phosphatidylserine, phosphatidylethanolamine, phosphatidylinositol, inositol phosphatide, or sphingomyelin is the phospholipid; isopropyl palmitate, isopropyl myristate, isopropyl linoleate, isopropyl oleate, ethyl laurate, or ethyl myristate is the fatty acid ester; and a linoleic, oleic, stearic acid, linolenic, palmitic, arachidonic, palmitoleic, myristic, eicosenoic, benehic, euricic, or lignoceric acid is the long-chain fatty acid or the long-chain fatty acid is obtained from safflower oil or almond oil.

44. A formulation for transdermal delivery of a medicament through the skin of a subject, the formulation comprising a therapeutically effective amount of a medicament and a penetrant portion,
wherein the penetrant portion comprises: a phospholipid, a fatty acid ester formed from a low molecular weight alcohol, and a long-chain fatty acids, and one or more of a viscosity-improving agent, a penetration enhancer, and an emulsifier,
wherein phosphatidylcholine, hydrogenated phosphatidylcholine, phosphatidylserine, phosphatidylethanolamine, phosphatidylinositol, inositol phosphatide, or sphingomyelin is the phospholipid; isopropyl palmitate, isopropyl myristate, isopropyl linoleate, isopropyl oleate, ethyl laurate, or ethyl myristate is the fatty acid ester; and a linoleic, oleic, stearic acid, linolenic, palmitic, arachidonic, palmitoleic, myristic, eicosenoic, benehic, euricic, or lignoceric acid is the long-chain fatty acid or the long-chain fatty acid is obtained from safflower oil or almond oil; polyglyceryl-4-laurate, polyglyceryl-4-oleate, span 60, cetyl alcohol, or polyglyceryl-3-oleate is the penetration enhancer; a poloxamer (e.g., poloxamer 407, poloxamer 188, poloxamer 184, and poloxamer 124) or sodium lauryl sulfate is the viscosity-improving agent; benzyl alcohol, ethanol, propylene glycol, polyethylene glycol, limonene, menthol, borneol, or camphor is the penetration enhancer.

45. The formulation of any one of claims 1 to 44, wherein the penetrant portion is in an amount from about 70% to about 98% w/w of the formulation.

46. The formulation of any one of claims 1 to 45, wherein the penetrant portion comprises water.

47. The formulation of any one of claims 1 to 46, wherein the penetrant portion comprises water in an amount from about 50% to about 80% w/w of the formulation.

48. The formulation of any one of claims 1 to 47, wherein the formulation comprises a phospholipid, an emollient/moisturizer, a fatty acid, an alcohol, an oil, a surfactant, water, and a medicament.

49. A formulation for transdermal delivery of a medicament through the skin of a subject, the formulation comprising a phospholipid in an amount from about 5% to about 15% w/w of the formulation; an emollient/moisturizer in an amount from about 10% to about 20% w/w of the formulation; a fatty acid in an amount from about 0.5% to about 2% w/w of the formulation; an alcohol in an amount from about 0.5% to about 2% w/w of the formulation; an oil in an amount from about 1% to about 5% w/w of the formulation; a surfactant in an amount from about 0.5% to about 2% w/w of the formulation; water in an amount from about 30% to about 80% w/w of the formulation; and a therapeutically effective amount of a medicament in an amount from about 0.00001% to about 30% w/w of the formulation.

50. The formulation of claim 49, wherein the medicament is in an amount from about 0.00001% to about 0.01% w/w of the formulation.

51. The formulation of claim 49, wherein the medicament is in an amount from about 0.01% to about 0.1% w/w of the formulation.

52. The formulation of claim 49, wherein the medicament is in an amount from about 0.1% to about 1.0% w/w of the formulation.

53. The formulation of claim 49, wherein the medicament is in an amount from about 1% to about 10% w/w of the formulation.

54. The formulation of claim 49, wherein the medicament is in an amount from about 11% to about 20% w/w of the formulation.

55. The formulation of claim 49, wherein the medicament is in an amount from about 21% to about 30% w/w of the formulation.

56. A formulation for transdermal delivery of a medicament through the skin of a subject, the formulation comprising phosphatidylcholine in an amount of about 7.64% w/w of the formulation; isopropyl palmitate in an amount of about 13.30% w/w of the formulation; stearic acid in an amount of about 0.62% w/w of the formulation; benzyl alcohol in an amount of about 1.39% w/w of the formulation; safflower oil in an amount of about 2.93% w/w of the formulation; oleic acid in an amount of about 0.97% w/w of the formulation; polyglyceryl-4 laurate in an amount of about 1.06% w/w of the formulation; deionized water in an amount of about 60.84% w/w of the formulation; poloxamer 407 in an amount of about 9.25% w/w of the formulation; and a therapeutically effective amount of a medicament in an amount of about 2% w/w of the formulation.

57. A formulation for transdermal delivery of a medicament through the skin of a subject, the formulation comprising phosphatidylcholine in an amount of about 7.66% w/w of the formulation; isopropyl palmitate in an amount of about 13.34% w/w of the formulation; benzyl alcohol in an amount of about 1.39% w/w of the formulation; stearic acid in an amount of about 0.68% w/w of the formulation; carthamus tinctorius (safflower) oil in an amount of about 2.79% w/w of the formulation; polyglyceryl-4 laurate in an amount of about 1.07% w/w of the formulation; oleic acid in an amount of about 1.06% w/w of the formulation; deionized water in an amount of about 61.73% w/w of the formulation; poloxamer 407 in an amount of about 9.28% w/w of the formulation; and a therapeutically effective amount of a medicament in an amount of about 1.00% w/w of the formulation.

58. The formulation of claim 56 or claim 57, wherein rather than the medicament being in an amount of about 1% or about 2% w/w of the formulation, the amount of the medicament is less than about 1% and the amount of water is increased proportionally.

59. The formulation of claim 56 or claim 57, wherein rather than the medicament being in an amount of about 1% or about 2% w/w of the formulation, the amount of the medicament is greater than about 2% and the amount of water is decreased proportionally.

60. The formulation of claim 59, wherein the amount of the medicament is less than about 30%.

61. A formulation for transdermal delivery of a medicament through the skin of a subject, the formulation comprising a therapeutically effective amount of a medicament and a penetrant portion, wherein the penetrant portion comprises: phosphatidylcholine in an amount from about 3% to about 15% w/w of the formulation; isopropyl palmitate in an amount from about 5% to about 20% w/w of the formulation; stearic acid in an amount from about 0.1% to about 10% w/w of the formulation; benzyl alcohol in an amount from about 0.5% to about 5% w/w of the formulation; polyglyceryl-4 laurate in an amount from about 0.5% to about 10% w/w of the formulation; and poloxamer 407 in an amount from about 5% to about 20% w/w of the formulation.

62. The formulation of any one of claims 1 to 61, wherein the formulation has a pH from about 7 to about 10.5.

63. The formulation of any one of claims 1 to 62, wherein the formulation has a pH from about 9 to about 11.

64. The formulation of any one of claims 1 to 63, wherein the medicament has a molecular weight of less than about 500 Da, has a molecular weight from about 500 Da to about 1000 Da, or has a molecular weight greater than about 1000 Da, e.g., greater than about 10,000 Da.

65. The formulation of any one of claims 1 to 64, wherein the one or more medicaments is selected from Table 1 or comprises one or more buffering agents as disclosed herein.

66. The formulation of claim 65, wherein the amount of the one or more medicaments is the effective dose of the medicament as described in Table 1 or the effective dose of the buffering agents as disclosed herein.

67. The formulation of any one of claims 1 to 66 wherein the medicament is a buffering agent.

68. The formulation of claim 67, wherein the buffering agent is Sodium Hydroxide (Sodium oxidanide), Sodium Bicarbonate (baking soda or Sodium hydrogen carbonate), Potassium Bicarbonate (potassium hydrogen carbonate or potassium acid carbonate), Lysine, Tris (Tromethamine, trisaminomethane, 2-amino-2-hydroxymethyl-propane-1,3-diol, or tris(hydroxymethyl)aminomethane), Calcium Carbonate, Sodium Carbonate (Disodium carbonate), Potassium Carbonate, Dipotassium Phosphate (Potassium phosphate dibasic or Potassium hydrogen phosphate), Disodium Phosphate (Sodium phosphate dibasic or Disodium hydrogen phosphate), Trisodium Phosphate, Meglumine ((2R,3R,4R,5S)-6-(Methylamino)hexane-1,2,3,4,5-pentol or Methylglucamine), Arginine, Triethanolamine (TEA or 2,2′,2″-Nitrilotriethanol), Glycine, Monosodium Phosphate (Sodium dihydrogen phosphate), Monopotassium Phosphate (Potassium dihydrogen phosphate), Tripotassium Phosphate (potassium phosphate), Monoethanolamine, Diethanolamine (Diolamine or 2-(2-hydroxyethylamino)ethanol), Magnesium carbonate, 2-imidazole-1-yl-3-ethoxycarbonylpropionic acid (IEPA), or combination thereof.

69. The formulation of any one of claims 1 to 68, wherein transdermal delivery provides systemic administration of the medicament.

70. A method for transdermal delivery of a buffering agent for treating a local and/or superficial disease or disorder, the method comprising a step of applying to the skin of a subject an effective amount of the transdermal delivery formulation of any one of claims 67 to 69.

71. The method of claim 70, wherein local and/or superficial disease or disorder is relative to skin surface.

72. The method of claim 70 or claim 71, wherein the local and/or superficial disease or disorder comprises inflammation of the skin.

73. The method of any one of claims 70 to 72, wherein the local and/or superficial disease or disorder is ankylosing spondylitis, gout, dermatomyositis, juvenile idiopathic arthritis, atopic dermatitis, psoriasis, seborrheic dermatitis, urticaria, hidradenitis suppurativa, folliculitis, neuritis, otitis externa, otitis media, capillaritis, bursitis, tendinitis, chondritis, sinusitis, rhinitis, pharyngitis, laryngitis, tracheitis, bronchitis, pleuritis, mediastinitis, cellulitis, synovitis, myositis, enthesitis, fasciitis, capsulitis, epicondylitis, panniculitis, osteomyelitis, spondylitis, periostitis, vaginitis, vulvitis, mastitis, orchitis, sudden onset hearing loss, pruritus, seborrhea, nasal polyps, contact dermatitis, neurodermatitis, prurigo, bullous pemphigoid, keloids, hypertrophic scars, palmoplantar pustulosis, erythema multiforme, Duhring's disease, epidermolysis bullosa, or a combination thereof.

74. A method for transdermal delivery of a buffering agent for treating a local, non-superficial disease or disorder, the method comprising a step of applying to the skin of a subject an effective amount of the transdermal delivery formulation of any one of claims 67 to 69.

75. The method of claim 74, wherein local, non-superficial disease or disorder is relative to skin surface.

76. The method of claim 74 or claim 75, wherein the local, non-superficial disease or disorder comprises inflammation of an organ.

77. The method of any one of claims 74 to 76, wherein the local, non-superficial disease or disorder is endometriosis, asthma, autoimmune encephalitis, osteomyelitis, lupus, scleroderma, vasculitis, myelitis, myositis, Sjogren's syndrome, ulcerative colitis, familial mediterranean fever, neonatal onset multisystem inflammatory disease, tumor necrosis factor receptor-associated periodic syndrome, Behcet's disease, glomerulonephritis, hepatitis, endocarditis, myocarditis, pericarditis, appendicitis, Crohn's disease, amyloidosis, Still's disease, pelvic inflammatory disease, arteritis, gastritis, pancreatitis, peritonitis, cholecystitis, ureteritis, cystitis, urethritis, oophoritis, salpingitis, parametritis, cervicitis, prostatitis, hypophysitis, thyroiditis, parathyroiditis, adrenalitis, lymphangitis, lymphadenitis, rheumatoid arthritis, psoriatic arthritis, reactive arthritis, allergic rhinitis, sarcoidosis, atherosclerosis, hypertension, left ventricular hypertropy or a combination thereof.

78. A method for transdermal delivery of a buffering agent for treating a local, non-superficial disease or disorder, the method comprising a step of applying to the skin of a subject an effective amount of the transdermal delivery formulation of any one of claims 67 to 69.

79. The method of claim 78, wherein transdermal delivery provides systemic administration of the medicament.

80. The method of claim 78 or claim 79, wherein the local, non-superficial disease or disorder is fatty liver disease, NASH, diabetes mellitus, chronic kidney disease, inflammatory bowel disease, Kawasaki disease, systemic inflammatory response syndrome, multiple sclerosis, cardiovascular disease, Alzheimer's disease, Parkinson's disease, or a combination thereof.

81. The method of any one of claims 67 to 80, wherein the medicament comprises a non-sodium buffering agent to help avoid hypernatremia in a subject.

82. The method of any one of claims 67 to 80, wherein the medicament comprises both a non-sodium buffering agent and a sodium buffering agent, wherein the sodium buffering agent is in an amount less than the non-sodium buffering agent to help avoid hypernatremia in a subject.

83. The method of claim 82, wherein, the amount of the sodium buffering agent is 1%, 2%, 3%, 4%, 5%, 6%, 7%, 8%, 9%, 10%, 20%, 30%, 40%, 50%, 60%, 70%, of the amount of the non-sodium sodium buffering agent.

84. A method for transdermal delivery of a buffering agent for treating a RAS/MAPK driven tumor, the method comprising a step of applying to the skin of a subject an effective amount of the transdermal delivery formulation of any one of claims 67 to 69.

85. The method of claim 84, wherein the method further comprises administering naporafenib via intravenous injection or oral dosage.

86. The method of claim 84, wherein the transdermal delivery formulation comprises naporafenib.

87. The method of any one of claims 84 to 86, wherein the method further comprises administering a chemotherapeutic agent.

88. The method of claim 87, wherein the chemotherapeutic agent is administered before, contemporary with, or after the transdermal delivery formulation is administered.

89. The method of claim 87 or claim 88, wherein the chemotherapeutic agent is directed to RAS/MAPK driven tumors.

90. The method of any one of claims 84 to 89, wherein the RAS/MAPK driven tumor is melanoma, unresectable metastatic melanoma, solid tumors, non-small cell lunger cancer, metastatic BRAF V600 colorectal cancer, pancreatic cancer, colorectal cancer, non-small cell lung cancer, acute myeloid leukemia, melanoma, bladder, thyroid, seminoma, liver diseases or disorders, kidney diseases or disorders, myelodysplastic syndrome, acute myelogenous leukemia, melanoma, hairy cell, ovarian cancer, breast cancer, or a combination thereof.

91. The method of any one of claims 84 to 90, wherein the RAS/MAPK driven tumor is melanoma, unresectable metastatic melanoma, solid tumors, non-small cell lunger cancer, metastatic BRAF V600 colorectal cancer, or combinations thereof.

92. The method of any one of claims 84 to 91, wherein the RAS/MAPK driven tumor is pancreatic cancer, colorectal cancer, non-small cell lung cancer, acute myeloid leukemia, melanoma, bladder, thyroid, seminoma, liver diseases or disorders, kidney diseases or disorders, myelodysplastic syndrome, acute myelogenous leukemia, melanoma, hairy cell, ovarian cancer, breast cancer, or a combination thereof.

93. A method for transdermally delivering at least one medicament, the method comprising a step of applying to the skin of a subject an effective amount of the formulation of any one of claims 1 to 69.

94. A method for treating a disease or disorder or reducing a symptom thereof, the method comprising:
administering to a subject in need thereof a transdermal delivery formulation of any one of claims 1 to 69 and administering to the subject in need thereof a composition comprising one or more medicaments selected from Table 1.

95. The method of claim 94, wherein the transdermal delivery formulation is administered before, contemporary with, or after the composition is administered.

96. The method of claim 94 or claim 95, wherein the amount of the one or more medicaments is the effective dose of the medicament as described in Table 1.

97. The method of any one of claims 94 to 96, wherein the composition is administered by the standard route for the medicament.

98. The method of claim 97, wherein the standard route is oral, topical, enteral, parenteral, by intravenous injection or infusion, by intraperitoneal injection, by intramuscular injection, or by subcutaneous injection.

99. The method of claim 97 or claim 98, wherein the composition is a liquid, a suspension, a gel, a geltab, a semisolid, a tablet, a sachet, a lozenge, a pill, or a capsule.

100. Use of a transdermal delivery formulation of any one of claims 1 to 69 in a method for treating a disease or disorder or reducing a symptom thereof.

101. A method for manufacturing a medicament for treating a disease or disorder or reducing a symptom thereof comprising combining a penetrant portion as recited in any one of claims 1 to 63 with one or more medicaments recited in any one of claims 64 to 69.

102. A method of treating cancer comprising:
applying the transdermal delivery formulation of claim 1, wherein the formulation comprises a buffering agent, to a skin of a subject;
increasing a pH of a tumor microenvironment;
increasing one or more of: translation of a proinflammatory cytokines T cell activation;
perfusion, or activity, of an immunotherapy, immunomodulatory agent, or chemotherapeutic agent within the tumor microenvironment; and
decreasing one or more of: tumor size, or inhibition of glycolytic activity.

103. The method of claim 102, wherein the buffering agent is Sodium Hydroxide (Sodium oxidanide), Sodium Bicarbonate (baking soda or Sodium hydrogen carbonate), Potassium Bicarbonate (potassium hydrogen carbonate or potassium acid carbonate), Lysine, Tris (Tromethamine, trisaminomethane, 2-amino-2-hydroxymethyl-propane-1,3-diol, or tris(hydroxymethyl)aminomethane), Calcium Carbonate, Sodium Carbonate (Disodium carbonate), Potassium Carbonate, Dipotassium Phosphate (Potassium phosphate dibasic or Potassium hydrogen phosphate), Disodium Phosphate (Sodium phosphate dibasic or Disodium hydrogen phosphate), Trisodium Phosphate, Meglumine ((2R,3R,4R,5S)-6-(Methylamino)hexane-1,2,3,4,5-pentol or Methylglucamine), Arginine, Triethanolamine (TEA or 2,2′,2″-Nitrilotriethanol), Glycine, Monosodium Phosphate (Sodium dihydrogen phosphate), Monopotassium Phosphate (Potassium dihydrogen phosphate), Tripotassium Phosphate (potassium phosphate), Monoethanolamine, Diethanolamine (Diolamine or 2-(2-hydroxyethylamino)ethanol), Magnesium carbonate, 2-imidazole-1-yl-3-ethoxycarbonylpropionic acid (IEPA), or combination thereof.

104. The method of any one of claim 102 or claim 103, wherein the cancer or the tumor microenvironment is near the surface of the skin.

105. The method of the any one of claims 102 to 104 wherein the cancer or the tumor microenvironment comprises a melanoma.

106. The method of any one of claims 104 to 105, wherein the method further comprises administering an anti-inflammatory agent.

107. The method of claim 106, wherein the anti-inflammatory agent is administered before, contemporary with, or after the transdermal delivery formulation is administered.

108. The method of claim 106 or claim 107, wherein the transdermal delivery formulation synergizes the effect from the anti-inflammatory agent and/or the anti-inflammatory agent synergizes the effect from the transdermal delivery formulation.

109. The method of any one of claims 106 to 108, wherein the dosage of the anti-inflammatory agent is reduced when administered before, contemporary with, or after the transdermal delivery formulation is administered relative to its dosage when administered alone.

110. The method of claim 109, wherein the reduced dosage of the anti-inflammatory agent improves improve safety of the subject.

111. The method of any one of claims 106 to 110, wherein the anti-inflammatory agent is hydrocortisone, hydrocortisone acetate, cortisone acetate, tixocortol pivalate, prednisolone, methylprednisolone, prednisone, amcinonide, budesonide, desonide, fluocinolone acetonide, fluocinonide, halcinonide, triamcinolone acetonide, beclometasone, betamethasone, dexamethasone, fluocortolone, halometasone, mometasone, ciclesonide, cortisone acetate, hydrocortisone aceponate, hydrocortisone acetate, hydrocortisone buteprate, hydrocortisone butyrate, hydrocortisone valerate, prednicarbate, or tixocortol pivalate.

112. The method of any one of claims 106 to 110, wherein the anti-inflammatory agent is acetylsalicylic acid, diflunisal, salicylic acid and its salts, salsalate, Ibuprofen, Dexibuprofen, Naproxen, Fenoprofen, Ketoprofen, Dexketoprofen, Flurbiprofen, Oxaprozin, Loxoprofen, Pelubiprofen, Zaltoprofen, Indomethacin, Tolmetin, Sulindac, Etodolac, Ketorolac, Diclofenac, Aceclofenac, Bromfenac, Nabumetone, Piroxicam, Meloxicam, Tenoxicam, Droxicam, Lornoxicam, Isoxicam, Phenylbutazone, Mefenamic acid, Meclofenamic acid, Flufenamic acid, Tolfenamic acid, Celecoxib, Refocoxib, Valdecoxib, Parecoxib, Lumiracoxic, Etoricoxib, Firocoxib, Nimesulide, Clonixin, or Licofelone.

113. A method of treating or preventing a symptom of an EGFR inhibitor induced side effect, the method comprising:
applying the transdermal delivery formulation of any one of claims 1 to 69, wherein the formulation comprises LUT014, to a skin of a subject;
binding BRAF with LUT014 or a metabolite thereof, thereby inhibiting an MAP Kinase pathway; and
treating or preventing the symptom of the EGFR inhibitor induced side effect.

114. The method of claim 113, wherein the EGFR inhibitor induced side effect comprises acneiform lesions.

115. A method of treating or preventing a symptom of radiation dermatitis, the method comprising:
applying the transdermal delivery formulation of any one of claims 1 to 69, wherein the formulation comprises LUT014, to a skin of a subject;
binding BRAF with LUT014 or a metabolite thereof, thereby increasing a prefoliation of a keratinocyte;
and treating or preventing the symptom of radiation dermatitis.

116. The method of claim 115 wherein treating or preventing the symptom of radiation dermatitis comprises decreasing in collagen production, improving skin elasticity, reducing telangiectasia, reducing redness, reducing inflammation, reducing swelling, reducing blisters or skin ulcers, reducing thinning or weakening of the skin, or combinations thereof.

117. A method of improving wound healing, the method comprising:
applying the transdermal delivery formulation of any one of claims 1 to 69, wherein the formulation comprises LUT014, to a skin of a subject;
binding BRAF with LUT014 or a metabolite thereof, thereby inhibiting an MAP Kinase pathway; and
improving wound healing.

118. A method of vaccinating a subject, the method comprising:
applying the transdermal delivery formulation of any one of claims 1 to 69, wherein the formulation comprises a nucleic acid, to a skin of a subject.

119. The method of claim 118, wherein the nucleic acid is an mRNA molecule which encodes a polypeptide or protein.
